SEQUENCE LISTING (231 sequences)

SEQ ID NO: 1 - protein, 1368 aa, Streptococcus pyogenes [sequence omitted]

SEQ ID NO: 2 - skipped

SEQ ID NO: 3 - protein, 1246 aa, Unknown
  Description of Unknown: Lachnospiraceae bacterium ND2006 sequence
MLKNVGIDRLDVEKGRKNMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 4 - protein, 1228 aa, Unknown
  Description of Unknown: Lachnospiraceae bacterium ND2006 sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 5 - protein, 4 aa, Artificial Sequence
  Description of Artificial Sequence: Synthetic peptide
GGGS

SEQ ID NO: 6 - protein, 5 aa, Artificial Sequence
  Description of Artificial Sequence: Synthetic peptide
GGGGS

SEQ ID NO: 7 - protein, 7 aa, Artificial Sequence
  Description of Artificial Sequence: Synthetic peptide
PKKKRRV

SEQ ID NO: 8 - protein, 16 aa, Artificial Sequence
  Description of Artificial Sequence: Synthetic peptide
KRPAATKKAGQAKKKK

SEQ ID NO: 9 - DNA, 5625 bp, Artificial Sequence
  Description of Artificial Sequence: Synthetic polynucleotide
atggcgccgaaaaaaaaacgcaaagtgaacggcggagggtccggaggaggcggtagcggaggcgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatgccattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcaggaggcggtggaagcgggggaaggggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggttctaggggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaagggggaagcggtggaagcgggaggggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggttctaggggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggataa

SEQ ID NO: 10 - DNA, 4443 bp, Artificial Sequence
  Description of Artificial Sequence: Synthetic polynucleotide
atggcgccgaaaaaaaaacgcaaagtgaacggcggagggtccggaggaggcggtagcggaggcgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatgccattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcaggaggcggtggaagcgggcgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgtta

SEQ ID NO: 11 - DNA, 5094 bp, Artificial Sequence
  Description of Artificial Sequence: Synthetic polynucleotide
atggcgccgaaaaaaaaacgcaaagtgaacggcggagggtccggaggaggcggtagcggaggcgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatgccattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcaggaggcggtggaagcgggatggagttccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgataactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcttaa

SEQ ID NO: 12 - DNA, 5928 bp, Artificial Sequence
  Description of Artificial Sequence: Synthetic polynucleotide
atggcgccgaaaaaaaaacgcaaagtgaacggcggagggtccggaggaggcggtagcggaggcgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatgccattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtcaaaaggccggcggccacgaaaaaggccggccaggcaaaaaagaaaaagggatcctacccatacgatgttccagattacgcttatccctacgacgtgcctgattatgcatacccatatgatgtccccgactatgccggaagcgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttaa

SEQ ID NO: 13 - DNA, 579 bp, Artificial Sequence
  Description of Artificial Sequence: Synthetic polynucleotide
atgggatccagaatcctctggcatgagatgtggcatgaaggcctggaagaggcatctcgtttgtactttggggaaaggaacgtgaaaggcatgtttgaggtgctggagcccttgcatgctatgatggaacggggaccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagggcggcggatcccccaagaagaagaggaaagtctcgagcgactacaaagaccatgacggtgattataaagatcatgacatcgattacaaggatgacgatgacaaggctgcaggaggcggtggaagcgggcgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttataa

SEQ ID NO: 14 - DNA, 6069 bp, Artificial Sequence
  Description of Artificial Sequence: Synthetic polynucleotide
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgacggtaccgcgggcccgggatccaccggtcgccaccatgggatccagaatcctctggcatgagatgtggcatgaaggcctggaagaggcatctcgtttgtactttggggaaaggaacgtgaaaggcatgtttgaggtgctggagcccttgcatgctatgatggaacggggaccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagggcggcggatcccccaagaagaagaggaaagtctcgagcgactacaaagaccatgacggtgattataaagatcatgacatcgattacaaggatgacgatgacaaggctgcaggaggcggtggaagcgggatggagttccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgataactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcttaaagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 15 - DNA, 6860 bp, Artificial Sequence
  Description of Artificial Sequence: Synthetic polynucleotide
ggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggsggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgacggtaccgcgggcccgggatccaccggtcgccaccatgggatccagaatcctctggcatgagatgtggcatgaaggcctggaagaggcatctcgtttgtactttggggaaaggaacgtgaaaggcatgtttgaggtgctggagcccttgcatgctatgatggaacggggaccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagggcggcggatcccccaagaagaagaggaaagtctcgagcgactacaaagaccatgacggtgattataaagatcatgacatcgattacaaggatgacgatgacaaggctgcaggaggcggtggaagcgggtcggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttaaagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 16 - DNA, 2280 bp, Artificial Sequence
  Description of Artificial Sequence: Synthetic polynucleotide
cgaggtacctctctacatatgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgctagctgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgattaatgcgtctccgttttagagctagaaatagcaagttaaaataaggctagtccgttatcaacttgaaaaagtggcaccgagtcggtgctttttttaagcttgggccgct

SEQ ID NO: 17 - DNA, 20 bp, Artificial Sequence
  Description of Artificial Sequence: Synthetic oligonucleotide
ctagtacaggttataagcct

SEQ ID NO: 18 - DNA, 20 bp, Artificial Sequence
  Description of Artificial Sequence: Synthetic oligonucleotide
tcttcagctacgcccataaa

SEQ ID NO: 19 - DNA, 20 bp, Artificial Sequence
  Description of Artificial Sequence: Synthetic oligonucleotide
ttatgggcgtagctgaagaa

SEQ ID NO: 20 - DNA, 20 bp, Artificial Sequence
  Description of Artificial Sequence: Synthetic oligonucleotide
ataagctgagtcctcccctc

SEQ ID NO: 21 - DNA, 20 bp, Artificial Sequence
  Description of Artificial Sequence: Synthetic oligonucleotide
tctgaaggaggtatctattt

SEQ ID NO: 22 - DNA, 20 bp, Artificial Sequence
  Description of Artificial Sequence: Synthetic oligonucleotide
gtgggtgattctgtgggcag

SEQ ID NO: 23 - DNA, 20 bp, Artificial Sequence
  Description of Artificial Sequence: Synthetic oligonucleotide
actgtgagcgtcctcagtgc

SEQ ID NO: 24 - DNA, 20 bp, Artificial Sequence
  Description of Artificial Sequence: Synthetic oligonucleotide
ccacgctcccagaaagcaaa

SEQ ID NO: 25 - DNA, 20 bp, Artificial Sequence
  Description of Artificial Sequence: Synthetic oligonucleotide
tcacaactgtaaggtgtagc

SEQ ID NO: 26 - DNA, 20 bp, Artificial Sequence
  Description of Artificial Sequence: Synthetic oligonucleotide
tatctaatggttgttaggga

SEQ ID NO: 27 - DNA, 20 bp, Artificial Sequence
  Description of Artificial Sequence: Synthetic oligonucleotide
caatgtatagtgtgttgttg

SEQ ID NO: 28 - DNA, 20 bp, Artificial Sequence
  Description of Artificial Sequence: Synthetic oligonucleotide
tcaagcaagtaggcggcaag

SEQ ID NO: 29 - DNA, 20 bp, Artificial Sequence
  Description of Artificial Sequence: Synthetic oligonucleotide
ctgagaaaatagtatagccg

SEQ ID NO: 30 - DNA, 20 bp, Artificial Sequence
  Description of Artificial Sequence: Synthetic oligonucleotide
tcctttctgacgtctcaccc

SEQ ID NO: 31 - DNA, 20 bp, Artificial Sequence
  Description of Artificial Sequence: Synthetic oligonucleotide
gcctgggctccggggcgttt

SEQ ID NO: 32 - DNA, 20 bp, Artificial Sequence
  Description of Artificial Sequence: Synthetic oligonucleotide
gggcccctgcggccaccccg

SEQ ID NO: 33 - DNA, 20 bp, Artificial Sequence
  Description of Artificial Sequence: Synthetic oligonucleotide
cctccctccctgcccggtag

SEQ ID NO: 34 - DNA, 20 bp, Artificial Sequence
  Description of Artificial Sequence: Synthetic oligonucleotide
gaggtttggaaagggcgtgc

SEQ ID NO: 35 - DNA, 20 bp, Artificial Sequence
  Description of Artificial Sequence: Synthetic oligonucleotide
ccaactgagtcctgaggttt

SEQ ID NO: 36 - DNA, 20 bp, Artificial Sequence
  Description of Artificial Sequence: Synthetic oligonucleotide
actcacagcacagccagtgt

SEQ ID NO: 37 - DNA, 20 bp, Artificial Sequence
  Description of Artificial Sequence: Synthetic oligonucleotide
ccagcagctggtcacaaagc

SEQ ID NO: 38 - DNA, 20 bp, Artificial Sequence
  Description of Artificial Sequence: Synthetic oligonucleotide
ccttcctataaacttctgag

SEQ ID NO: 39 - DNA, 20 bp, Artificial Sequence
  Description of Artificial Sequence: Synthetic oligonucleotide
cttggggtgattccctagag

SEQ ID NO: 40 - DNA, 20 bp, Artificial Sequence
  Description of Artificial Sequence: Synthetic oligonucleotide
gctactatcacaagcctgtg

SEQ ID NO: 41 - DNA, 20 bp, Artificial Sequence
  Description of Artificial Sequence: Synthetic oligonucleotide
gctactatcacaagcctgtg

SEQ ID NO: 42 - DNA, 20 bp, Artificial Sequence
  Description of Artificial Sequence: Synthetic oligonucleotide
ggctcttctggcactggctt

SEQ ID NO: 43 - DNA, 20 bp, Artificial Sequence
  Description of Artificial Sequence: Synthetic oligonucleotide
gaaggttacacagaaccaga

SEQ ID NO: 44 - DNA, 20 bp, Artificial Sequence
  Description of Artificial Sequence: Synthetic oligonucleotide
tgacctttgcccagcgccct

SEQ ID NO: 45 - DNA, 20 bp, Artificial Sequence
  Description of Artificial Sequence: Synthetic oligonucleotide
ctgcggggagtcggtggtcc

SEQ ID NO: 46 - DNA, 20 bp, Artificial Sequence
  Description of Artificial Sequence: Synthetic oligonucleotide
gcatctgaccccaacaatca

SEQ ID NO: 47 - DNA, 20 bp, Artificial Sequence
  Description of Artificial Sequence: Synthetic oligonucleotide
tgcaacaatgtctggcacat

SEQ ID NO: 48 - DNA, 20 bp, Artificial Sequence
  Description of Artificial Sequence: Synthetic oligonucleotide
ccagggaggtgagccttgca

SEQ ID NO: 49 - DNA, 20 bp, Artificial Sequence
  Description of Artificial Sequence: Synthetic oligonucleotide
ctctcacatgctagtggcac

SEQ ID NO: 50 - DNA, 20 bp, Artificial Sequence
  Description of Artificial Sequence: Synthetic oligonucleotide
tccccgccacgttgaaaggc

SEQ ID NO: 51 - DNA, 20 bp, Artificial Sequence
  Description of Artificial Sequence: Synthetic oligonucleotide
caggcctttcatgcccacta

SEQ ID NO: 52 - DNA, 20 bp, Artificial Sequence
  Description of Artificial Sequence: Synthetic oligonucleotide
ggctcggtcccgcgtcgtcg

SEQ ID NO: 53 - DNA, 22 bp, Artificial Sequence
  Description of Artificial Sequence: Synthetic primer
gagacttcctgcctcgtcacaa

SEQ ID NO: 54 - DNA, 22 bp, Artificial Sequence
  Description of Artificial Sequence: Synthetic primer
gctggacttcagcccaaaatgc

SEQ ID NO: 55 - DNA, 21 bp, Artificial Sequence
  Description of Artificial Sequence: Synthetic primer
ctccaactgctccgacggcat

SEQ ID NO: 56 - DNA, 20 bp, Artificial Sequence
  Description of Artificial Sequence: Synthetic primer
tcactagcaagctctcaggc

SEQ ID NO: 57 - DNA, 21 bp, Artificial Sequence
  Description of Artificial Sequence: Synthetic primer
gctgagtgaactgcactgtga

SEQ ID NO: 58 - DNA, 20 bp, Artificial Sequence
  Description of Artificial Sequence: Synthetic primer
gcacgtggatcctgagaact

SEQ ID NO: 59 - DNA, 19 bp, Artificial Sequence
  Description of Artificial Sequence: Synthetic primer
ctctgcccgagcttcagag

SEQ ID NO: 60 - DNA, 22 bp, Artificial Sequence
  Description of Artificial Sequence: Synthetic primer
gatcagcaggaaaacacagccg

SEQ ID NO: 61 - DNA, 22 bp, Artificial Sequence
  Description of Artificial Sequence: Synthetic primer
agtccaacccagtgttcctctc

SEQ ID NO: 62 - DNA, 22 bp, Artificial Sequence
  Description of Artificial Sequence: Synthetic primer
acaggcagtctaggctcgacac

SEQ ID NO: 63 - DNA, 19 bp, Artificial Sequence
  Description of Artificial Sequence: Synthetic primer
aacaacgaggagtctgccc

SEQ ID NO: 64 - DNA, 20 bp, Artificial Sequence
  Description of Artificial Sequence: Synthetic primer
gaattctttgccgaaatgga

SEQ ID NO: 65 - DNA, 21 bp, Artificial Sequence
  Description of Artificial Sequence: Synthetic primer
attggacagcaagaaagcgag

SEQ ID NO: 66 - DNA, 20 bp, Artificial Sequence
  Description of Artificial Sequence: Synthetic primer
tgtccttaacggtgctccag

SEQ ID NO: 67 - DNA, 20 bp, Artificial Sequence
  Description of Artificial Sequence: Synthetic primer
ctccttgttgttgccctcct

SEQ ID NO: 68 - DNA, 20 bp, Artificial Sequence
  Description of Artificial Sequence: Synthetic primer
ctccttgttgttgccctcct

SEQ ID NO: 69 - DNA, 25 bp, Artificial Sequence
  Description of Artificial Sequence: Synthetic primer
ggtcttggtggcgtgcttcatgtta

SEQ ID NO: 70 - DNA, 25 bp, Artificial Sequence
  Description of Artificial Sequence: Synthetic primer
ggtcttggtggcgtgcttcatgttg

SEQ ID NO: 71 - DNA, 20 bp, Artificial Sequence
  Description of Artificial Sequence: Synthetic primer
atgctgcctcctttttgatg

SEQ ID NO: 72 - DNA, 20 bp, Artificial Sequence
  Description of Artificial Sequence: Synthetic primer
gtgcttgcaacaatgtctgg

SEQ ID NO: 73 - DNA, 20 bp, Artificial Sequence
  Description of Artificial Sequence: Synthetic primer
gtgcttgcaacaatgtctgg

SEQ ID NO: 74 - DNA, 20 bp, Artificial Sequence
  Description of Artificial Sequence: Synthetic primer
ggtgccactagcatgtgaga

SEQ ID NO: 75 - DNA, 20 bp, Artificial Sequence
  Description of Artificial Sequence: Synthetic primer
atttgagcatgtccgagagc

SEQ ID NO: 76 - DNA, 20 bp, Artificial Sequence
  Description of Artificial Sequence: Synthetic primer
agggtgcaagtagctgatgg

SEQ ID NO: 77 - DNA, 18 bp, Artificial Sequence
  Description of Artificial Sequence: Synthetic primer
atctcagccccgagaagg

SEQ ID NO: 78 - DNA, 20 bp, Artificial Sequence
  Description of Artificial Sequence: Synthetic primer
atctccactggtcagcaggt

SEQ ID NO: 79 - DNA, 25 bp, Artificial Sequence
  Description of Artificial Sequence: Synthetic primer
aaccctgcttatcttaaaccaacct

SEQ ID NO: 80 - DNA, 20 bp, Artificial Sequence
  Description of Artificial Sequence: Synthetic primer
agccttgtcctcctctgtga

SEQ ID NO: 81 - DNA, 20 bp, Artificial Sequence
  Description of Artificial Sequence: Synthetic primer
gtacgtgttggagcctggat

SEQ ID NO: 82 - DNA, 20 bp, Artificial Sequence
  Description of Artificial Sequence: Synthetic primer
ggggcactgagtactgacct

SEQ ID NO: 83 - DNA, 20 bp, Artificial Sequence
  Description of Artificial Sequence: Synthetic primer
ggggcactgagtactgacct

SEQ ID NO: 84 - DNA, 20 bp, Artificial Sequence
  Description of Artificial Sequence: Synthetic primer
cacacactctggtggatgct

SEQ ID NO: 85 - DNA, 20 bp, Artificial Sequence
  Description of Artificial Sequence: Synthetic primer
tgatgcaaaacgtaccctca

SEQ ID NO: 86 - DNA, 20 bp, Artificial Sequence
  Description of Artificial Sequence: Synthetic primer
tgcctgttgcacagataagg

SEQ ID NO: 87 - DNA, 20 bp, Artificial Sequence
  Description of Artificial Sequence: Synthetic primer
caactgggaggaacaaggtc

SEQ ID NO: 88 - DNA, 20 bp, Artificial Sequence
  Description of Artificial Sequence: Synthetic primer
cagctgcctctagggatgaa

SEQ ID NO: 89 - DNA, 20 bp, Artificial Sequence
  Description of Artificial Sequence: Synthetic primer
ctctgccctgccttttatgc

SEQ ID NO: 90 - DNA, 20 bp, Artificial Sequence
  Description of Artificial Sequence: Synthetic primer
aaacggtccctggctaaact

SEQ ID NO: 91 - DNA, 20 bp, Artificial Sequence
  Description of Artificial Sequence: Synthetic primer
gcctcctttttgatgcagcc

SEQ ID NO: 92 - DNA, 19 bp, Artificial Sequence
  Description of Artificial Sequence: Synthetic primer
atccttggcggtcttggtg

SEQ ID NO: 93 - DNA, 53 bp, Artificial Sequence
  Description of Artificial Sequence: Synthetic primer
acactctttccctacacgacgctcttccgatctgcctcctttttgatgcagcc

SEQ ID NO: 94 - DNA, 53 bp, Artificial Sequence
  Description of Artificial Sequence: Synthetic primer
acactctttccctacacgacgctcttccgatctgcttgcaacaatgtctggca

SEQ ID NO: 95 - DNA, 53 bp, Artificial Sequence; identical to SEQ ID NO: 94
  Description of Artificial Sequence: Synthetic primer

SEQ ID NO: 96 - DNA, 53 bp, Artificial Sequence
  Description of Artificial Sequence: Synthetic primer
acactctttccctacacgacgctcttccgatctgaatcaggcacagtccagct

SEQ ID NO: 97 - DNA, 53 bp, Artificial Sequence
  Description of Artificial Sequence: Synthetic primer
acactctttccctacacgacgctcttccgatcttgagcatgtccgagagcatc

SEQ ID NO: 98 - DNA, 53 bp, Artificial Sequence
  Description of Artificial Sequence: Synthetic primer
acactctttccctacacgacgctcttccgatctgcagggagagaatgagagcc

SEQ ID NO: 99 - DNA, 51 bp, Artificial Sequence
  Description of Artificial Sequence: Synthetic primer
acactctttccctacacgacgctcttccgatctatctcagccccgagaagg

SEQ ID NO: 100 - DNA, 53 bp, Artificial Sequence
  Description of Artificial Sequence: Synthetic primer
acactctttccctacacgacgctcttccgatctgttcctgagctcatctgggc

SEQ ID NO: 101 - DNA, 53 bp, Artificial Sequence
  Description of Artificial Sequence: Synthetic primer
acactctttccctacacgacgctcttccgatctactccttgttgttgccctcc

SEQ ID NO: 102 - DNA, 52 bp, Artificial Sequence
  Description of Artificial Sequence: Synthetic primer
acactctttccctacacgacgctcttccgatctctctgcccgagcttcagag

SEQ ID NO: 103 - DNA, 53 bp, Artificial Sequence
  Description of Artificial Sequence: Synthetic primer
acactctttccctacacgacgctcttccgatctatgctgcctcctttttgatg

SEQ ID NO: 104 - DNA, 53 bp, Artificial Sequence
  Description of Artificial Sequence: Synthetic primer
acactctttccctacacgacgctcttccgatctcgtactgcctgtgtgtcctt

SEQ ID NO: 105 - DNA, 53 bp, Artificial Sequence
  Description of Artificial Sequence: Synthetic primer
acactctttccctacacgacgctcttccgatctgtgcttgcaacaatgtctgg

SEQ ID NO: 106 - DNA, 53 bp, Artificial Sequence
  Description of Artificial Sequence: Synthetic primer
acactctttccctacacgacgctcttccgatctagaggggaggaggagactga

SEQ ID NO: 107 - DNA, 53 bp, Artificial Sequence
  Description of Artificial Sequence: Synthetic primer
acactctttccctacacgacgctcttccgatctatttgagcatgtccgagagc

SEQ ID NO: 108 - DNA, 53 bp, Artificial Sequence
  Description of Artificial Sequence: Synthetic primer
acactctttccctacacgacgctcttccgatctagggtgcaagtagctgatgg

SEQ ID NO: 109 - DNA, 51 bp, Artificial Sequence; identical to SEQ ID NO: 99
  Description of Artificial Sequence: Synthetic primer

SEQ ID NO: 110 - DNA, 53 bp, Artificial Sequence
  Description of Artificial Sequence: Synthetic primer
acactctttccctacacgacgctcttccgatctctcatctccactggtcagca

SEQ ID NO: 111 - DNA, 52 bp, Artificial Sequence
  Description of Artificial Sequence: Synthetic primer
gactggagttcagacgtgtgctcttccgatctgccagacattgttgcaagca

SEQ ID NO: 112 - DNA, 52 bp, Artificial Sequence
  Description of Artificial Sequence: Synthetic primer
gactggagttcagacgtgtgctcttccgatctggctgaggtgtcattcgtga

SEQ ID NO: 113 - DNA, 52 bp, Artificial Sequence; identical to SEQ ID NO: 112
  Description of Artificial Sequence: Synthetic primer

SEQ ID NO: 114 - DNA, 52 bp, Artificial Sequence
  Description of Artificial Sequence: Synthetic primer
gactggagttcagacgtgtgctcttccgatctcggacatgctcaaatggtgc

SEQ ID NO: 115 - DNA, 52 bp, Artificial Sequence
  Description of Artificial Sequence: Synthetic primer
gactggagttcagacgtgtgctcttccgatctggctctcattctctccctgc

SEQ ID NO: 116 - DNA, 52 bp, Artificial Sequence
  Description of Artificial Sequence: Synthetic primer
gactggagttcagacgtgtgctcttccgatctatcagcctcattgcctcacc

SEQ ID NO: 117 - DNA, 52 bp, Artificial Sequence
  Description of Artificial Sequence: Synthetic primer
gactggagttcagacgtgtgctcttccgatctgagcaactgggaggaacaag

SEQ ID NO: 118 - DNA, 52 bp, Artificial Sequence
  Description of Artificial Sequence: Synthetic primer
gactggagttcagacgtgtgctcttccgatcttgaactgagcagacaggcag

SEQ ID NO: 119 - DNA, 51 bp, Artificial Sequence
  Description of Artificial Sequence: Synthetic primer
gactggagttcagacgtgtgctcttccgatctatccttggcggtcttggtg

SEQ ID NO: 120 - DNA, 52 bp, Artificial Sequence
  Description of Artificial Sequence: Synthetic primer
gactggagttcagacgtgtgctcttccgatcttgtccttaacggtgctccag

SEQ ID NO: 121 - DNA, 52 bp, Artificial Sequence
  Description of Artificial Sequence: Synthetic primer
gactggagttcagacgtgtgctcttccgatctgtacgtgttggagcctggat

SEQ ID NO: 122 - DNA, 52 bp, Artificial Sequence
  Description of Artificial Sequence: Synthetic primer
gactggagttcagacgtgtgctcttccgatctagctggactgtgcctgattc

SEQ ID NO: 123 - DNA, 52 bp, Artificial Sequence
  Description of Artificial Sequence: Synthetic primer
gactggagttcagacgtgtgctcttccgatctggggcactgagtactgacct

SEQ ID NO: 124 - DNA, 52 bp, Artificial Sequence
  Description of Artificial Sequence: Synthetic primer
gactggagttcagacgtgtgctcttccgatctcacacactctggtggatgct

SEQ ID NO: 125 - DNA, 52 bp, Artificial Sequence
  Description of Artificial Sequence: Synthetic primer
gactggagttcagacgtgtgctcttccgatcttgatgcaaaacgtaccctca

SEQ ID NO: 126 - DNA, 52 bp, Artificial Sequence
  Description of Artificial Sequence: Synthetic primer
gactggagttcagacgtgtgctcttccgatcttgcctgttgcacagataagg

SEQ ID NO: 127 - DNA, 52 bp, Artificial Sequence; identical to SEQ ID NO: 117
  Description of Artificial Sequence: Synthetic primer

SEQ ID NO: 128 - DNA, 52 bp, Artificial Sequence
  Description of Artificial Sequence: Synthetic primer
gactggagttcagacgtgtgctcttccgatctactgagcagacaggcaggag

SEQ ID NO: 129 - DNA, 8 bp, Artificial Sequence
  Description of Artificial Sequence: Synthetic oligonucleotide
tatagcct

SEQ ID NO: 130 - DNA, 8 bp, Artificial Sequence
  Description of Artificial Sequence: Synthetic oligonucleotide
atagaggc

SEQ ID NO: 131 - DNA, 8 bp, Artificial Sequence
  Description of Artificial Sequence: Synthetic oligonucleotide
cctatcct

SEQ ID NO: 132 - DNA, 8 bp, Artificial Sequence
  Description of Artificial Sequence: Synthetic oligonucleotide
ggctctga

SEQ ID NO: 133 - DNA, 8 bp, Artificial Sequence
  Description of Artificial Sequence: Synthetic oligonucleotide
aggcgaag

SEQ ID NO: 134 - DNA, 8 bp, Artificial Sequence
  Description of Artificial Sequence: Synthetic oligonucleotide
taatctta

SEQ ID NO: 135 - DNA, 8 bp, Artificial Sequence
  Description of Artificial Sequence: Synthetic oligonucleotide
caggacgt

SEQ ID NO: 136 - DNA, 8 bp, Artificial Sequence
  Description of Artificial Sequence: Synthetic oligonucleotide
gtactgac

SEQ ID NO: 137 - DNA, 8 bp, Artificial Sequence
  Description of Artificial Sequence: Synthetic oligonucleotide
aacggttg

SEQ ID NO: 138 - DNA, 8 bp, Artificial Sequence
  Description of Artificial Sequence: Synthetic oligonucleotide
ctgttcta

SEQ ID NO: 139 - DNA, 8 bp, Artificial Sequence
  Description of Artificial Sequence: Synthetic oligonucleotide
cattgtaa

SEQ ID NO: 140 - DNA, 8 bp, Artificial Sequence
  Description of Artificial Sequence: Synthetic oligonucleotide
ctcctcga

SEQ ID NO: 141 - DNA, 8 bp, Artificial Sequence
  Description of Artificial Sequence: Synthetic oligonucleotide
actcgcct

SEQ ID NO: 142 - DNA, 8 bp, Artificial Sequence
  Description of Artificial Sequence: Synthetic oligonucleotide
cagttgtt

SEQ ID NO: 143 - DNA, 8 bp, Artificial Sequence
  Description of Artificial Sequence: Synthetic oligonucleotide
agagatcc

SEQ ID NO: 144 - DNA, 8 bp, Artificial Sequence
  Description of Artificial Sequence: Synthetic oligonucleotide
tggcactt

SEQ ID NO: 145 - DNA, 70 bp, Artificial Sequence
  Description of Artificial Sequence: Synthetic oligonucleotide
aatgatacggcgaccaccgagatctacactatagcctacactctttccctacacgacgctcttccgatct

SEQ ID NO: 146 - DNA, 70 bp, Artificial Sequence
  Description of Artificial Sequence: Synthetic oligonucleotide
aatgatacggcgaccaccgagatctacacatagaggcacactctttccctacacgacgctcttccgatct

SEQ ID NO: 147 - DNA, 70 bp, Artificial Sequence
  Description of Artificial Sequence: Synthetic oligonucleotide
aatgatacggcgaccaccgagatctacaccctatcctacactctttccctacacgacgctcttccgatct

SEQ ID NO: 148 - DNA, 70 bp, Artificial Sequence
  Description of Artificial Sequence: Synthetic oligonucleotide
aatgatacggcgaccaccgagatctacacggctctgaacactctttccctacacgacgctcttccgatct

SEQ ID NO: 149 - DNA, 70 bp, Artificial Sequence
  Description of Artificial Sequence: Synthetic oligonucleotide
aatgatacggcgaccaccgagatctacacaggcgaagacactctttccctacacgacgctcttccgatct

SEQ ID NO: 150 - DNA, 70 bp, Artificial Sequence
  Description of Artificial Sequence: Synthetic oligonucleotide
aatgatacggcgaccaccgagatctacactaatcttaacactctttccctacacgacgctcttccgatct

SEQ ID NO: 151 - DNA, 70 bp, Artificial Sequence
  Description of Artificial Sequence: Synthetic oligonucleotide
aatgatacggcgaccaccgagatctacaccaggacgtacactctttccctacacgacgctcttccgatct

SEQ ID NO: 152 - DNA, 70 bp, Artificial Sequence
  Description of Artificial Sequence: Synthetic oligonucleotide
aatgatacggcgaccaccgagatctacacgtactgacacactctttccctacacgacgctcttccgatct

SEQ ID NO: 153 - DNA, 70 bp, Artificial Sequence
  Description of Artificial Sequence: Synthetic oligonucleotide
aatgatacggcgaccaccgagatctacacaacggttgacactctttccctacacgacgctcttccgatct

SEQ ID NO: 154 - DNA, 70 bp, Artificial Sequence
  Description of Artificial Sequence: Synthetic oligonucleotide
aatgatacggcgaccaccgagatctacacctgttctaacactctttccctacacgacgctcttccgatct

SEQ ID NO: 155 - DNA, 70 bp, Artificial Sequence
  Description of Artificial Sequence: Synthetic oligonucleotide
aatgatacggcgaccaccgagatctacaccattgtaaacactctttccctacacgacgctcttccgatct

SEQ ID NO: 156 - DNA, 70 bp, Artificial Sequence
  Description of Artificial Sequence: Synthetic oligonucleotide
aatgatacggcgaccaccgagatctacacctcctcgaacactctttccctacacgacgctcttccgatct

SEQ ID NO: 157 - DNA, 70 bp, Artificial Sequence
  Description of Artificial Sequence: Synthetic oligonucleotide
aatgatacggcgaccaccgagatctacacactcgcctacactctttccctacacgacgctcttccgatct

SEQ ID NO: 158 - DNA, 70 bp, Artificial Sequence
  Description of Artificial Sequence: Synthetic oligonucleotide
aatgatacggcgaccaccgagatctacaccagttgttacactctttccctacacgacgctcttccgatct

SEQ ID NO: 159 - DNA, 70 bp, Artificial Sequence
  Description of Artificial Sequence: Synthetic oligonucleotide
aatgatacggcgaccaccgagatctacacagagatccacactctttccctacacgacgctcttccgatct

SEQ ID NO: 160 - DNA, 70 bp, Artificial Sequence
  Description of Artificial Sequence: Synthetic oligonucleotide
aatgatacggcgaccaccgagatctacactggcacttacactctttccctacacgacgctcttccgatct

SEQ ID NO: 161 - DNA, 8 bp, Artificial Sequence
  Description of Artificial Sequence: Synthetic oligonucleotide
attactcg

SEQ ID NO: 162 - DNA, 8 bp, Artificial Sequence
  Description of Artificial Sequence: Synthetic oligonucleotide
tccggaga

SEQ ID NO: 163 - DNA, 8 bp, Artificial Sequence
  Description of Artificial Sequence: Synthetic oligonucleotide
cgctcatt

SEQ ID NO: 164 - DNA, 8 bp, Artificial Sequence
  Description of Artificial Sequence: Synthetic oligonucleotide
gagattcc

SEQ ID NO: 165 - DNA, 8 bp, Artificial Sequence
  Description of Artificial Sequence: Synthetic oligonucleotide
attcagaa

SEQ ID NO: 166 - DNA, 8 bp, Artificial Sequence
  Description of Artificial Sequence: Synthetic oligonucleotide
gaattcgt

SEQ ID NO: 167 - DNA, 8 bp, Artificial Sequence
  Description of Artificial Sequence: Synthetic oligonucleotide
ctgaagct

SEQ ID NO: 168 - DNA, 8 bp, Artificial Sequence
  Description of Artificial Sequence: Synthetic oligonucleotide
taatgcgc

SEQ ID NO: 169 - DNA, 8 bp, Artificial Sequence
  Description of Artificial Sequence: Synthetic oligonucleotide
cggctatg

SEQ ID NO: 170 - DNA, 8 bp, Artificial Sequence
  Description of Artificial Sequence: Synthetic oligonucleotide
tccgcgaa

SEQ ID NO: 171 - DNA, 8 bp, Artificial Sequence
  Description of Artificial Sequence: Synthetic oligonucleotide
tctcgcgc

SEQ ID NO: 172 - DNA, 8 bp, Artificial Sequence
  Description of Artificial Sequence: Synthetic oligonucleotide
agcgatag

SEQ ID NO: 173 - DNA, 8 bp, Artificial Sequence
  Description of Artificial Sequence: Synthetic oligonucleotide
atcctatc

SEQ ID NO: 174 - DNA, 8 bp, Artificial Sequence
  Description of Artificial Sequence: Synthetic oligonucleotide
tcgtgtca

SEQ ID NO: 175 - DNA, 8 bp, Artificial Sequence
  Description of Artificial Sequence: Synthetic oligonucleotide
ctgtacta

SEQ ID NO: 176 - DNA, 8 bp, Artificial Sequence
  Description of Artificial Sequence: Synthetic oligonucleotide
gtcgtcta

SEQ ID NO: 177 - DNA, 8 bp, Artificial Sequence
  Description of Artificial Sequence: Synthetic oligonucleotide
gtattcca

SEQ ID NO: 178 - DNA, 8 bp, Artificial Sequence
  Description of Artificial Sequence: Synthetic oligonucleotide
ggcagacc

SEQ ID NO: 179 - DNA, 8 bp, Artificial Sequence
  Description of Artificial Sequence: Synthetic oligonucleotide
agtataat

SEQ ID NO: 180 - DNA, 8 bp, Artificial Sequence
  Description of Artificial Sequence: Synthetic oligonucleotide
gaacgtgc

SEQ ID NO: 181 - DNA, 8 bp, Artificial Sequence
  Description of Artificial Sequence: Synthetic oligonucleotide
gtggagat

SEQ ID NO: 182 - DNA, 8 bp, Artificial Sequence
  Description of Artificial Sequence: Synthetic oligonucleotide
tgtatctt

SEQ ID NO: 183 - DNA, 8 bp, Artificial Sequence
  Description of Artificial Sequence: Synthetic oligonucleotide
atctggaa

SEQ ID NO: 184 - DNA, 8 bp, Artificial Sequence
  Description of Artificial Sequence: Synthetic oligonucleotide
taaggagg

SEQ ID NO: 185 - DNA, 66 bp, Artificial Sequence
  Description of Artificial Sequence: Synthetic oligonucleotide
caagcagaagacggcatacgagatcgagtaatgtgactggagttcagacgtgtgctcttccgatct

SEQ ID NO: 186 - DNA, 66 bp, Artificial Sequence
  Description of Artificial Sequence: Synthetic oligonucleotide
caagcagaagacggcatacgagattctccggagtgactggagttcagacgtgtgctcttccgatct

SEQ ID NO: 187 - DNA, 66 bp, Artificial Sequence
  Description of Artificial Sequence: Synthetic oligonucleotide
caagcagaagacggcatacgagataatgagcggtgactggagttcagacgtgtgctcttccgatct

SEQ ID NO: 188 - DNA, 66 bp, Artificial Sequence
  Description of Artificial Sequence: Synthetic oligonucleotide
caagcagaagacggcatacgagatggaatctcgtgactggagttcagacgtgtgctcttccgatct

SEQ ID NO: 189 - DNA, 66 bp, Artificial Sequence
  Description of Artificial Sequence: Synthetic oligonucleotide
caagcagaagacggcatacgagatttctgaatgtgactggagttcagacgtgtgctcttccgatct

SEQ ID NO: 190 - DNA, 66 bp, Artificial Sequence
  Description of Artificial Sequence: Synthetic oligonucleotide
caagcagaagacggcatacgagatacgaattcgtgactggagttcagacgtgtgctcttccgatct

SEQ ID NO: 191 - DNA, 66 bp, Artificial Sequence
  Description of Artificial Sequence: Synthetic oligonucleotide
caagcagaagacggcatacgagatagcttcaggtgactggagttcagacgtgtgctcttccgatct

SEQ ID NO: 192 - DNA, 66 bp, Artificial Sequence
  Description of Artificial Sequence: Synthetic oligonucleotide
caagcagaagacggcatacgagatgcgcattagtgactggagttcagacgtgtgctcttccgatct

SEQ ID NO: 193 - DNA, 66 bp, Artificial Sequence
  Description of Artificial Sequence: Synthetic oligonucleotide
caagcagaagacggcatacgagatcatagccggtgactggagttcagacgtgtgctcttccgatct

SEQ ID NO: 194 - DNA, 66 bp, Artificial Sequence
  Description of Artificial Sequence: Synthetic oligonucleotide
caagcagaagacggcatacgagatttcgcggagtgactggagttcagacgtgtgctcttccgatct

SEQ ID NO: 195 - DNA, 66 bp, Artificial Sequence
  Description of Artificial Sequence: Synthetic oligonucleotide
caagcagaagacggcatacgagatgcgcgagagtgactggagttcagacgtgtgctcttccgatct

SEQ ID NO: 196 - DNA, 66 bp, Artificial Sequence
  Description of Artificial Sequence: Synthetic oligonucleotide
caagcagaagacggcatacgagatctatcgctgtgactggagttcagacgtgtgctcttccgatct

SEQ ID NO: 197 - DNA, 66 bp, Artificial Sequence
  Description of Artificial Sequence: Synthetic oligonucleotide
caagcagaagacggcatacgagatgataggatgtgactggagttcagacgtgtgctcttccgatct

SEQ ID NO: 198 - DNA, 66 bp, Artificial Sequence
  Description of Artificial Sequence: Synthetic oligonucleotide
caagcagaagacggcatacgagattgacacgagtgactggagttcagacgtgtgctcttccgatct

SEQ ID NO: 199 - DNA, 66 bp, Artificial Sequence
  Description of Artificial Sequence: Synthetic oligonucleotide
caagcagaagacggcatacgagattagtacaggtgactggagttcagacgtgtgctcttccgatct

SEQ ID NO: 200 - DNA, 66 bp, Artificial Sequence
  Description of Artificial Sequence: Synthetic oligonucleotide
caagcagaagacggcatacgagattagacgacgtgactggagttcagacgtgtgctcttccgatct

SEQ ID NO: 201 - DNA, 66 bp, Artificial Sequence
  Description of Artificial Sequence: Synthetic oligonucleotide
caagcagaagacggcatacgagattggaatacgtgactggagttcagacgtgtgctcttccgatct

SEQ ID NO: 202 - DNA, 66 bp, Artificial Sequence
  Description of Artificial Sequence: Synthetic oligonucleotide
caagcagaagacggcatacgagatggtctgccgtgactggagttcagacgtgtgctcttccgatct

SEQ ID NO: 203 - DNA, 66 bp, Artificial Sequence
  Description of Artificial Sequence: Synthetic oligonucleotide
caagcagaagacggcatacgagatattatactgtgactggagttcagacgtgtgctcttccgatct

SEQ ID NO: 204 - DNA, 66 bp, Artificial Sequence
  Description of Artificial Sequence: Synthetic oligonucleotide
caagcagaagacggcatacgagatgcacgttcgtgactggagttcagacgtgtgctcttccgatct

SEQ ID NO: 205 - DNA, 66 bp, Artificial Sequence
  Description of Artificial Sequence: Synthetic oligonucleotide
caagcagaagacggcatacgagatatctccacgtgactggagttcagacgtgtgctcttccgatct

SEQ ID NO: 206 - DNA, 66 bp, Artificial Sequence
  Description of Artificial Sequence: Synthetic oligonucleotide
caagcagaagacggcatacgagataagatacagtgactggagttcagacgtgtgctcttccgatct

SEQ ID NO: 207 - DNA, 66 bp, Artificial Sequence
  Description of Artificial Sequence: Synthetic oligonucleotide
caagcagaagacggcatacgagatttccagatgtgactggagttcagacgtgtgctcttccgatct

SEQ ID NO: 208 - DNA, 66 bp, Artificial Sequence
  Description of Artificial Sequence: Synthetic oligonucleotide
caagcagaagacggcatacgagatcctccttagtgactggagttcagacgtgtgctcttccgatct

SEQ ID NO: 209 - DNA, 262 bp, Artificial Sequence
  Description of Artificial Sequence: Synthetic polynucleotide
  Description of Combined DNA/RNA Molecule: Synthetic polynucleotide
  modified_base (1)..(20): a, c, t, or g
  misc_feature (1)..(20): This region may encompass 17-20 nucleotides
  modified_base (63)..(262): a, c, u, g, unknown or other
  misc_feature (63)..(262): This region may encompass 0-200 nucleotides
nnnnnnnnnnnnnnnnnnnnguuuuagagcuagaaauagcaaguuaaaauaaggcuaguccgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 210 - DNA, 275 bp, Artificial Sequence
  Description of Artificial Sequence: Synthetic polynucleotide
  Description of Combined DNA/RNA Molecule: Synthetic polynucleotide
  modified_base (1)..(20): a, c, t, or g
  misc_feature (1)..(20): This region may encompass 17-20 nucleotides
  modified_base (76)..(275): a, c, u, g, unknown or other
  misc_feature (76)..(275): This region may encompass 0-200 nucleotides
nnnnnnnnnnnnnnnnnnnnguuuuagagcuaugcugaaaagcauagcaaguuaaaauaaggcuaguccguuaucnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 211 - DNA, 287 bp, Artificial Sequence
  Description of Artificial Sequence: Synthetic polynucleotide
  Description of Combined DNA/RNA Molecule: Synthetic polynucleotide
  modified_base (1)..(20): a, c, t, or g
  misc_feature (1)..(20): This region may encompass 17-20 nucleotides
  modified_base (88)..(287): a, c, u, g, unknown or other
  misc_feature (88)..(287): This region may encompass 0-200 nucleotides
nnnnnnnnnnnnnnnnnnnnguuuuagagcuaugcuguuuuggaaacaaaacagcauagcaaguuaaaauaaggcuaguccguuaucnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 212 - DNA, 296 bp, Artificial Sequence
  Description of Artificial Sequence: Synthetic polynucleotide
  Description of Combined DNA/RNA Molecule: Synthetic polynucleotide
  modified_base (1)..(20): a, c, t, or g
  misc_feature (1)..(20): This region may encompass 17-20 nucleotides
  modified_base (97)..(296): a, c, u, g, unknown or other
  misc_feature (97)..(296): This region may encompass 0-200 nucleotides
nnnnnnnnnnnnnnnnnnnnguuuuagagcuagaaauagcaaguuaaaauaaggcuaguccguuaucaacuugaaaaaguggcaccgagucggugcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 213 - DNA, 96 bp, Artificial Sequence
  Description of Artificial Sequence: Synthetic polynucleotide
  Description of Combined DNA/RNA Molecule: Synthetic polynucleotide
  modified_base (1)..(20): a, c, t, or g
  misc_feature (1)..(20): This region may encompass 17-20 nucleotides
nnnnnnnnnnnnnnnnnnnnguuuaagagcuagaaauagcaaguuuaaauaaggcuaguccguuaucaacuugaaaaaguggcaccgagucggugc

SEQ ID NO: 214 - DNA, 106 bp, Artificial Sequence
  Description of Artificial Sequence: Synthetic polynucleotide
  Description of Combined DNA/RNA Molecule: Synthetic polynucleotide
  modified_base (1)..(20): a, c, t, or g
  misc_feature (1)..(20): This region may encompass 17-20 nucleotides
nnnnnnnnnnnnnnnnnnnnguuuuagagcuaugcuggaaacagcauagcaaguuuaaauaaggcuaguccguuaucaacuugaaaaaguggcaccgagucggugc

SEQ ID NO: 215 - DNA, 106 bp, Artificial Sequence
  Description of Artificial Sequence: Synthetic polynucleotide
  Description of Combined DNA/RNA Molecule: Synthetic polynucleotide
  modified_base (1)..(20): a, c, t, or g
  misc_feature (1)..(20): This region may encompass 17-20 nucleotides
nnnnnnnnnnnnnnnnnnnnguuuaagagcuaugcuggaaacagcauagcaaguuuaaauaaggcuaguccguuaucaacuugaaaaaguggcaccgagucggugc

SEQ ID NO: 216 - DNA, 32 bp, Artificial Sequence
  Description of Artificial Sequence: Synthetic oligonucleotide
  Description of Combined DNA/RNA Molecule: Synthetic oligonucleotide
  modified_base (1)..(20): a, c, t, or g
  misc_feature (1)..(20): This region may encompass 17-20 nucleotides
nnnnnnnnnnnnnnnnnnnnguuuuagagcua

SEQ ID NO: 217 - DNA, 42 bp, Artificial Sequence
  Description of Artificial Sequence: Synthetic oligonucleotide
  Description of Combined DNA/RNA Molecule: Synthetic oligonucleotide
  modified_base (1)..(20): a, c, t, or g
  misc_feature (1)..(20): This region may encompass 17-20 nucleotides
nnnnnnnnnnnnnnnnnnnnguuuuagagcuaugcuguuuug

SEQ ID NO: 218 - DNA, 36 bp, Artificial Sequence
  Description of Artificial Sequence: Synthetic oligonucleotide
  Description of Combined DNA/RNA Molecule: Synthetic oligonucleotide
  modified_base (1)..(20): a, c, t, or g
  misc_feature (1)..(20): This region may encompass 17-20 nucleotides
nnnnnnnnnnnnnnnnnnnnguuuuagagcuaugcu

SEQ ID NO: 219 - RNA, 79 nt, Artificial Sequence
  Description of Artificial Sequence: Synthetic oligonucleotide
ggaaccauucaaaacagcauagcaaguuaaaauaaggcuaguccguuaucaacuugaaaaaguggcaccgagucggugc

SEQ ID NO: 220 - RNA, 60 nt, Artificial Sequence
  Description of Artificial Sequence: Synthetic oligonucleotide
uagcaaguuaaaauaaggcuaguccguuaucaacuugaaaaaguggcaccgagucggugc

SEQ ID NO: 221 - RNA, 64 nt, Artificial Sequence
  Description of Artificial Sequence: Synthetic oligonucleotide
agcauagcaaguuaaaauaaggcuaguccguuaucaacuugaaaaaguggcaccgagucggugc

SEQ ID NO: 222 - DNA, 42 bp, Artificial Sequence; identical to SEQ ID NO: 217
  Description of Artificial Sequence: Synthetic oligonucleotide
  Description of Combined DNA/RNA Molecule: Synthetic oligonucleotide
  modified_base (1)..(20): a, c, t, or g
  misc_feature (1)..(20): This region may encompass 17-20 nucleotides

SEQ ID NO: 223 - RNA, 79 nt, Artificial Sequence; identical to SEQ ID NO: 219
  Description of Artificial Sequence: Synthetic oligonucleotide

SEQ ID NO: 224 - DNA, 32 bp, Artificial Sequence; identical to SEQ ID NO: 216
  Description of Artificial Sequence: Synthetic oligonucleotide
  Description of Combined DNA/RNA Molecule: Synthetic oligonucleotide
  modified_base (1)..(20): a, c, t, or g
  misc_feature (1)..(20): This region may encompass 17-20 nucleotides

SEQ ID NO: 225 - RNA, 60 nt, Artificial Sequence; identical to SEQ ID NO: 220
  Description of Artificial Sequence: Synthetic oligonucleotide

SEQ ID NO: 226 - DNA, 36 bp, Artificial Sequence; identical to SEQ ID NO: 218
  Description of Artificial Sequence: Synthetic oligonucleotide
  Description of Combined DNA/RNA Molecule: Synthetic oligonucleotide
  modified_base (1)..(20): a, c, t, or g
  misc_feature (1)..(20): This region may encompass 17-20 nucleotides

SEQ ID NO: 227 - RNA, 64 nt, Artificial Sequence; identical to SEQ ID NO: 221
  Description of Artificial Sequence: Synthetic oligonucleotide

SEQ ID NO: 228 - protein, 1368 aa, Artificial Sequence
  Description of Artificial Sequence: Synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 229 - protein, 16 aa, Artificial Sequence
  Description of Artificial Sequence: Synthetic peptide
  MISC_FEATURE (1)..(16): This region may encompass 2-4 “Gly Gly Gly Ser” repeating units
  See specification as filed for detailed description of substitutions and preferred embodiments
GGGSGGGSGGGSGGGS

SEQ ID NO: 230 - protein, 20 aa, Artificial Sequence
  Description of Artificial Sequence: Synthetic peptide
  MISC_FEATURE (1)..(20): This region may encompass 2-4 “Gly Gly Gly Gly Ser” repeating units
  See specification as filed for detailed description of substitutions and preferred embodiments
GGGGSGGGGSGGGGSGGGGS

SEQ ID NO: 231 - protein, 6 aa, Artificial Sequence
  Description of Artificial Sequence: Synthetic 6xHis tag
HHHHHH

(19) United States
(12) Patent Application Publication
(10) Pub. No.: US 2023/0036273 A1
(43) Pub. Date: Feb. 2, 2023

(54) SYSTEM AND METHOD FOR ACTIVATING GENE EXPRESSION

(71) Applicant: The General Hospital Corporation, Boston, MA (US)

(72) Inventors: J. Keith Joung, Winchester, MA (US); Y. Esther Tak, Charlestown, MA (US)

(21) Appl. No.: 17/779,372

(22) PCT Filed: Nov. 25, 2020
(86) PCT No.: PCT/US2020/062166

§ 371 Date: May 24, 2022

Related U.S. Application Data
(60) Provisional application No. 62/941,334, filed on Nov. 27, 2019.

(51) Int. Cl. C07K 14/47; C12N 15/62

(52) U.S. Cl. CPC C07K 14/4702; C12N 15/62; C07K 2319/80

(57) ABSTRACT

Provided herein are artificial transcription factor systems and related methods for modulating gene expression.

### CLAIM OF PRIORITY

This application claims the benefit of U.S. Provisional Application Ser. No. 62/941,334, filed on Nov. 27, 2019. The entire contents of the foregoing are incorporated herein by reference.

### FEDERALLY SPONSORED RESEARCH OR DEVELOPMENT

This invention was made with Government support under Grant Nos. GM118158, CA211707, and CA204954 awarded by the National Institutes of Health. The Government has certain rights in the invention.

### TECHNICAL FIELD

The present application relates to methods and compositions for modulating gene expression.

### BACKGROUND

Epigenetic editing technologies enable efficient and tunable regulation of target gene expression for basic research, synthetic biology, and therapeutic applications. See Pickar et al., “The next generation of CRISPR-Cas technologies and applications,” Nat Rev Mol Cell Biol 20, 490-507, doi:10.1038/s41580-019-0131-5 (2019); Thakore et al., “Editing the epigenome: technologies for programmable transcription and epigenetic modulation,” Nat Methods 13, 127-137, doi:10.1038/nmeth.3733 (2016); and Wang et al., “CRISPR/Cas9 in Genome Editing and Beyond,” Annu Rev Biochem 85, 227-264, doi:10.1146/annurev-biochem-060815-014607 (2016). These strategies use artificial transcription factors (aTFs) composed of a gene regulatory effector domain fused to a programmable DNA-binding domain. In contrast to the multiple approaches currently available to repress gene expression (e.g., RNAi, anti-sense oligonucleotides), aTFs offer the distinguishing capability to activate gene expression. To date, gene expression modulation (e.g., transcriptional activation) has been primarily accomplished by directing aTFs to gene promoter-proximal sequences (less than +/−0.5 kb relative to the transcription start site (TSS)) rather than to more distally positioned enhancer sequences, which typically only show activity in a cell-type-specific fashion. Attempts to activate enhancers heterotopically (i.e., outside of their normal cell-type-specific context) by placing one or more aTFs at these sites have yielded only modest or no increases in gene transcription. See Hilton et al., “Epigenome editing by a CRISPR-Cas9-based acetyltransferase activates genes from promoters and enhancers,” Nat Biotechnol 33, 510-517, doi:10.1038/nbt.3199 (2015); and Benabdallah et al., “Decreased Enhancer-Promoter Proximity Accompanying Enhancer Activation,” Mol Cell, doi:10.1016/j.molcel.2019.07.038 (2019).

### SUMMARY

The present application is based, in part, on the discovery that directing artificial transcription factors (aTFs) targeted to both the enhancer regions and promoter regions of genes enable dynamic modulation of gene expression.

Thus, provided herein are artificial transcription factor (aTF) systems comprising:(a) one or more enhancer-targeting aTF(s); and (b) one or more promoter-targeting aTF(s).

In some embodiments, the enhancer-targeting aTF(s) comprise (a) a fusion protein comprising a catalytically inactive Cas9 or catalytically inactive Cpf1 and a gene expression modulating domain; and (b) a gRNA comprising a sequence complementary to a target gene enhancer sequence.

In some embodiments, the enhancer-targeting aTF(s) comprise (a) a first fusion protein comprising a catalytically inactive Cas9 or catalytically inactive Cpf1 and a first dimerization domain; (b) a second fusion protein comprising a gene expression modulating domain and a second dimerization domain; and (c) a gRNA comprising a sequence complementary to a target gene enhancer sequence.

In some embodiments, the promoter-targeting aTF(s) comprise (a) a fusion protein comprising a catalytically inactive Cas9 or catalytically inactive Cpf1 and a gene expression modulating domain; and (b) a gRNA comprising a sequence complementary to a target gene promoter sequence.

In some embodiments, the promoter-targeting aTF(s) comprise (a) a first fusion protein comprising a catalytically inactive Cas9 or catalytically inactive Cpf1 and a first dimerization domain; (b) a second fusion protein comprising a gene expression modulating domain and a second dimerization domain; and (c) a gRNA comprising a sequence complementary to a target gene promoter sequence.

Also provided herein is an artificial transcription factor (aTF) system comprising: (a) a fusion protein comprising a catalytically inactive Cas9 or catalytically inactive Cpf1 and a gene expression modulating domain; (b) a first gRNA comprising a sequence complementary to a target gene enhancer sequence; and (c) a second gRNA comprising a sequence complementary to a target gene promoter sequence.

Also provided herein is an artificial transcription factor (aTF) system comprising: (a) a first fusion protein comprising a catalytically inactive Cas9 or catalytically inactive Cpf1 and a first dimerization domain; (b) a second fusion protein comprising a gene expression modulating domain and a second dimerization domain; (c) a first gRNA comprising a sequence complementary to a target gene enhancer sequence; and (d) a second gRNA comprising a sequence complementary to a target gene promoter sequence.

Also provided herein is an artificial transcription factor (aTF) system comprising: (a) a fusion protein comprising a catalytically inactive Cas9 or catalytically inactive Cpf1 and a gene expression modulating domain; (b) a first gRNA comprising a sequence complementary to a target gene enhancer sequence; and (c) a plurality of gRNAs each comprising a sequence complementary to a different target gene promoter sequence.

Also provided herein is an artificial transcription factor (aTF) system comprising: (a) a first fusion protein comprising a catalytically inactive Cas9 or catalytically inactive Cpf1 and a first dimerization domain; (b) a second fusion protein comprising a gene expression modulating domain and a second dimerization domain; (c) a first gRNA comprising a sequence complementary to a target gene enhancer sequence; and (d) a plurality of gRNAs each comprising a sequence complementary to a different target gene promoter sequence.

In some embodiments, the first dimerization domain comprises DmrA and the second dimerization domain comprises DmrC.

In some embodiments, the aTF system further comprises a dimerization agent.

In some embodiments, the gene expression modulating domain is an activation domain selected from the group consisting of p65, VPR, VPR64, p300, and combinations thereof.

In some embodiments, the gene expression modulating domain comprises: (1) a protein that can introduce or remove covalent modifications to histones or DNA, optionally LSD1 or TET1; or (2) a protein that directly or indirectly recruits other proteins in the cell that in turn can modulate gene expression.

In some embodiments, the enhancer-targeting aTF, the promoter-targeting aTF, or both each comprises two or more gene expression modulating domains.

In some embodiments, the aTF system further comprises a drug that induces the activity of the enhancer-targeting aTF(s) and/or the promoter-targeting aTF(s).

In some embodiments, the target gene enhancer sequence comprises two or more alleles and the enhancer-targeting aTF comprises a programmable DNA binding domain specific for a subset of the alleles; and/or the target gene promoter sequence comprises two or more alleles and the promoter-targeting aTF comprises a programmable DNA binding domain specific for a subset of the alleles.

In some embodiments, the target gene enhancer sequence comprises two or more alleles and the gRNA is specific for a subset of the alleles; and/or the promoter gene enhancer sequence comprises two or more alleles and the gRNA is specific for a subset of the alleles.

In some embodiments, the target gene is selected from the group consisting of IL2RA, MYOD1, CD69, HBB, HBE, HBG1/2, APOC3, APOA4 and combinations thereof.

Also provided herein are vectors comprising nucleic acid sequences encoding one or more of the components of the aTF systems described herein.

Also provided herein are cells comprising the vectors described herein.

Also provided herein are pharmaceutical compositions comprising the aTF systems described herein and a pharmaceutically acceptable carrier.

Also provided herein are methods for modulating target gene expression in a cell comprising contacting the cell with any of the aTF systems, vectors, or pharmaceutical compositions described herein.

Also provided herein is a method for allele-specific modulation of a target gene expression in a cell comprising contacting the cell with any of the aTF systems, vectors, or pharmaceutical compositions described herein.

Also provided herein is a method for treating or preventing a condition or disease in a subject, comprising contacting the cell with any of the aTF systems, vectors, or pharmaceutical compositions described herein.

In some embodiments, the condition or disease is caused, at least in part, by insufficient expression of the target gene or the adverse effect of a mutant allele.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1H show robust heterotopic activation of enhancer sequences by Cas9-based aTFs in multiple human cell lines.

FIG. 1A schematically shows an enhancer X that activates promoter Y in cell type A (top line), the lack of enhancer X activity on promoter Y in a different cell type B (second line), lack of enhancer X activity on promoter Y in cell type B when an aTF is recruited only to enhancer X (third line), and robust enhancer X activity on promoter Y in cell type B when aTFs are recruited to both enhancer X and promoter Y (bottom line).

FIG. 1B schematically shows architectures of bi-partite and direct fusion dCas9-based aTFs used in this study.

FIGS. 1C-1E show RNA expression levels of the endogenous IL2RA (FIG. 1C), CD69 (FIG. 1D) and MYOD1 (FIG. 1E) genes in various indicated human cell lines in the presence of the bi-partite NF-KB p65 activator and one or more gRNAs targeting enhancer or promoter sequences. CD69 expression was not tested in K562 cells due to its high baseline expression in this cell line. gRNAs targeting the indicated enhancer sequences are denoted as E1, E2, E3, or E4 and gRNAs targeting the indicated promoter are indicated as P gRNAs. Transcript levels were measured by RT-qPCR, normalized to HPRT1 levels, and values shown are normalized relative to a control sample (labelled none) in which a gRNA targeting a sequence that does occur in the human genome' (hereafter, referred to as non-targeting) was expressed. Open circles indicate biological replicates (n=3), bars are the mean of replicates, and error represent the SD. * indicates results significantly different compared to the sample targeting only the promoter, p<0.05.

FIGS. 1F-1H show RNA expression levels of the endogenous IL2RA (FIG. 1F), CD69 (FIG. 1G) and MYOD1 (FIG. 1H) genes in various indicated human cell lines in the presence of various indicated bi-partite and direct fusion Cas9-based aTFs together with non-targeting gRNA (None), an enhancer-targeted gRNA (E only), a promoter-targeted gRNA (P only), or both (E+P). Enhancer gRNAs used for each experiment were those that showed optimal activity for each cell type from (FIGS. 1C-1E). Numbers shown in each box represent the mean fold-activation relative to the no gRNA (None) control for three biological replicates (n=3).

FIGS. 2A-2H show induction of allele-selective gene upregulation and expansion of the dynamic range of gene expression in human cells using heterotopic enhancer activation.

FIG. 2A shows schematic illustration of the human APOC3 gene and the two alleles of this locus present in HEK293 cells. E0 and P indicate gRNA binding sites in which NGG PAMs were intact in both alleles for an enhancer-targeted and promoter-targeted gRNA, respectively. E1-E6 indicate binding sites for gRNAs that lie within a potential enhancer region upstream of the known APOC3 enhancer and that were likely to preferentially target one allele or the other based on the identity of a SNP present in the PAMs of these target sites. A SNP in exon 3 of APOC3 that distinguishes the two alleles is also shown.

FIG. 2B shows binding to the potential upstream APOC3 enhancer sequence in HEK293 cells by the bi-partite NFKB p65 dCas9-based aTF in the presence of the E1-E6 gRNAs shown in FIG. 2A. Relative ratios of the two alleles quantified from next-generation sequencing of DNA amplified from ChIP-PCR experiments performed with an anti-Cas9 antibody are shown. Open circles indicate biological replicates (n=3), bars are the mean of replicates, and error bars represent SD.

FIG. 2C shows allelic ratios of APOC3 mRNA transcripts measured in HEK293 cells in which the bi-partite NF-KB p65 dCas9-based aTF was co-expressed with a promoter-targeted gRNA (P) either alone or with one or more gRNAs targeted to the APOC3 enhancer (EO) or upstream potential enhancer (E1-E6). Transcripts from the two alleles were quantified by next-generation sequencing of DNA amplified from the cDNA. Open circles indicate biological replicates (n=3), bars are the mean of replicates, and error represent SD.

FIG. 2D shows schematics illustrating genomic locations of enhancer-targeted gRNAs for the IL2RA, CD69, and MYOD1 genes previously shown to be optimal for activation for each gene in HEK293 cells (from FIG. 1(c-e)) and four promoter-targeted gRNAs designed for each gene.

FIG. 2E shows RNA expression levels of the endogenous IL2RA, CD69 and MYOD1 genes in HEK293 cells, as determined by RT-qPCR in the presence of the bi-partite NF-KB p65 activator and various combinations of the promoter- and enhancer-targeted gRNAs shown in FIG. 2D. A non-targeting gRNA was used instead of promoter-targeted gRNAs for the control samples (labelled as None). Open circles indicate biological replicates (n=3), bars are the mean of replicates, and error bars represent SD. * indicates expression levels significantly different from their matched sample lacking the enhancer-targeted gRNA (p<0.05).

FIG. 2F shows schematic of the human APOA4 and APOC3 genes and the two alleles of this locus present in HEK293 cells. E0 and P_{A4}/P_{C3} indicate binding sites for gRNAs targeting the known shared enhancer and the promoters, respectively. E1-E6 indicate binding sites for gRNAs targeting the potential enhancer regions, that are expected to preferentially target one allele over another based on whether the SNP present in the PAMs (NGG) of these target sites maintain or disrupt the PAMs. (Black bold underlined letters indicate bases that maintain an intact PAM site and gray bold underlined letters indicate bases that are expected to disrupt the PAM). Greyscale outlined boxes indicate PAMs targeted by E1-E6 on specific alleles, while black outlined boxes indicate PAMs targeted by E0, P_{A4}, P_{C3} on both alleles. The SNPs in exon 2 of APOA4 and exon 3 of APOC3 that distinguish between the mRNA of the two alleles are also shown.

FIG. 2G shows binding of the bi-partite p65 aTF to the potential upstream enhancer sequence in the presence of the E1-E6 gRNAs. E1, E2, and E4 are expected to bind selectively to Allele 1 (top); E3, E5, and E6 to Allele 2 (bottom). Relative quantification (percent next-generation sequencing reads) of the two alleles in the DNA from ChIP experiments performed with an anti-Cas9 antibody are shown. Open circles indicate biological replicates (n=3), bars the mean of replicates and error bars the s.e.m. In, Input DNA; Ch, Cas9 ChIP DNA

FIG. 2H shows relative quantification (percent next-generation sequencing reads of cDNA) of the two alleles of APOA3 and APOA4 mRNA when the bi-partite p65 aTF was co-expressed with a gRNA targeting the promoter (P_{A4} or P_{A3}) alone or with one or more gRNAs targeting the known enhancer (E0) or upstream potential enhancers (E1-E6). Open circles indicate biological replicates (n=3), bars the mean of replicates and error bars the s.e.m.

FIGS. 3A-3E show directing of heterotopic enhancer activities to a specific promoter in the human β-globin locus using dCas9-based aTFs.

FIG. 3A shows schematics illustrating normal developmental stage-specific activity of the locus control region (LCR) enhancer on expression of HBE, HBG1/2, and HBB in human erythroid cells. The LCR consists of five DNase hypersensitive sites (HS1-HS5) indicated by the grey peaks.

FIG. 3B shows genomic locations of gRNAs targeting the LCR HS2 region (E) and the promoter regions of HBE (P_{E}), HBG1/2(P_{G}), and HBB(P_{B}). P_{G} targets promoters of both HBG1 and HBG2 due to their high homology.

FIGS. 3C-3E show RNA expression levels of the HBE, HBG1/2, and HBB genes in various human cell lines in which the indicated bi-partite (FIG. 3C and FIG. 3D) or direct fusion (FIG. 3E) dCas9-based aTF was co-expressed with either a non-targeting gRNA (None), the LCR HS2 enhancer-targeted gRNA (E only), a promoter-targeted gRNA (P_{E}, P_{G}, or P_{B} only), or the E gRNA with one of the promoter-targeted gRNAs (E+P_{E}, E+P_{G}, or E+P_{B}). Relative expression of each gene was measured by RT-qPCR and normalized to HPRT1 levels, and the number in each box is the mean fold-activation relative to the None control of three biological replicates (n=3).

FIGS. 4A-4B show open and active chromatin status at IL2RA and MYOD1 determined by ATAC-seq and H3K27Ac ChIP-seq.

FIG. 4A shows IL2RA promoter was closed and inactive in all cell types, IL2RA enhancer region was closed and inactive in HEK293 and K562 cells, but open and active in U2OS and HepG2 cells. E1, E2, E3, E4: IL2RA enhancer gRNA target sites, P: IL2RA promoter gRNA target site. The RBM17 locus is open and active in all cell types.

FIG. 4B shows open chromatin at MYOD1 promoter in U2OS and HEK293 cells but not in HepG2 and K562 cells. E1, E2, E3, E4: MYOD1 enhancer gRNA target sites, P: MYOD1 promoter gRNA target site.

FIGS. 5A-5D show haplotype of APOC3 enhancer regions and allele ratios of target SNPs.

FIG. 5A shows genomic locations of SNPs identified in APOC3 potential enhancers, promoters and exon 3. Potential enhancer region has open chromatin features like known enhancer based on the DNase-seq and H3K27Ac data from HepG2 cells from the UCSC genome browser (hg19) in which APOC3 is highly expressed.

FIG. 5B shows Sanger sequencing traces of each SNP region described in FIG. 5A. E1 to E6 are gRNA binding sites in the potential enhancer regions that are next to PAMs in which targeted SNPs are present.

FIG. 5C shows allele ratios of target SNPs were identified by targeted genomic DNA amplicon sequencing and indicate a 1:1 ratio.

FIG. 5D shows Sanger sequencing traces from TOPO cloned amplicons showing the SNPs in the potential enhancer, promoter and exonic regions of APOA4 and APOC3 in HEK293 cells. E1 to E6 are gRNA binding sites in the potential enhancer regions which have SNPs in the PAM sequence. SNPs are exclusively associated with one another in two unique haplotypes.

FIGS. 6A-6C show allele-selective RT-qPCR targeting a APOC3 exonic SNP (rs4520).

FIG. 6A shows schematic of RT-qPCR primers for APOC3 expression. Allele-specific primers detecting a APOC3 exonic SNP have a common forward primer (P_{F_1}) which spans exon 2 and exon 3 junction, and two different reverse primers which are specific for allele 1 (T at rs4520, P_{R_1}) or for allele 2 (C at rs4520, P_{R_2}) in exon 3. Non-allele-specific primers (P_{F_2} and P_{R_3}) detect APOC3 expression from both alleles.

FIG. 6B shows allele-selective expression of APOC3 in HEK293 cells by bi-partite dCas9-based p65 aTF targeted to APOC3 promoter and various sites on the enhancer including SNP regions (E1 to E6) and non-SNP region (E0). RT-qPCR was performed using the primers described in FIG. 6A. Relative allele-selective expression of APOC3 was normalized to HPRT1 levels, and open circles indicate biological replicates (n=3).

FIG. 6C shows validation of the specificity of allele-specific RT-qPCR primers that detect the SNP in APOC3 exon 3 in HEK293 cells using U2OS cells in which variant nucleotide is absent (only C allele is present at the same position). Allele-selective expression of APOC3 in U2OS cells by bi-partite dCas9-based p65 aTF targeted to APOC3 promoter and non-SNP region (E0). APOC3 expression was measured using the same allele-specific primers and non-allele-specific primers used in FIG. 6B. Relative allele-selective and non-allele expression of APOC3 was normalized to HPRT1 levels, and open circles indicate biological replicates (n=3).

FIGS. 7A-7B show binding of bi-partite dCas9-based p65 aTF to APOC3 enhancer and promoter target sites in HEK293 cells.

FIG. 7A shows genomic locations of the enhancer gRNAs and APOC3 promoter gRNA. ChIP-qPCR amplicon regions are shown as boxes.

FIG. 7B shows binding activity of bi-partite dCas9-based p65 aTF on each gRNA target region in APOC3 locus determined by Cas9 ChIP-qPCR. Relative Cas9 ChIP-qPCR enrichment at each region was calculated over input DNA. Open circles indicate biological replicates (n=3).

FIG. 8 shows the impact of heterotopic enhancer activation on promoters of IL2RA, CD69, and MYOD1 at various levels of activation.

X-axis: the levels of promoter activation (fold-change in gene expression compared to the negative control) of target genes by bi-partite p65 activator and gRNAs that target promoters only. Y-axis: the effect of heterotopic activation by bi-partite p65 activator (fold-difference in gene expression between promoter activation alone and promoter with enhancer activation)

FIG. 9 shows open and active chromatin status at the β-globin locus determined by ATAC-seq and H3K27Ac ChIP-seq.

All promoters at the β-globin locus showed closed and inactive chromatin states in all cell types. HS2 enhancer region showed closed and inactive chromatin features in HEK293 cells, but open and active chromatin features U2OS and HepG2 cells. E: HS2 enhancer gRNA target sites, P_{E}: HBE promoter gRNA target site, P_{G}: HBG1/2 promoter gRNA target site, P_{B}: HBB promoter gRNA target site.

FIGS. 10A-10D show topologically associated domains (TADs) centered on the IL2RA (FIG. 10A), CD69 (FIG. 10B), MYOD1 (FIG. 10C), and APOC3 (FIG. 10D) loci from different cell types.

The IL2RA locus is located in the same TAD in various cell types. The triangle heatmaps for TADs were obtained from 3D genome browser^{35,36}.

FIGS. 11A-11B show distribution of SNP densities that create or disrupt NGG PAM sequences at putative enhancers and promoters.

FIG. 11A: The X-axis shows two categories of regulatory elements. The Y-axis shows the density of SNPs that create or disrupt NGG PAM sequences at each regulatory element.

FIG. 11B: The X-axis shows three categories of SNPs in PAM sequences; 1) creating PAM, 2) disrupting PAM, and 3) both creating and disrupting PAMs at the same time but on different strands. The Y-axis shows the density of SNPs of each category at each regulatory element. Y-axis value is the number of SNPs divided by the base pair size of each regulatory element.

### DETAILED DESCRIPTION

The present application is based, in part, on the discovery that directing artificial transcription factors (aTFs) to both the enhancer regions and promoter regions of genes enables synergistic and dynamic modulation of gene expression by both regulatory regions.

Thus, described herein are artificial transcription factor systems and methods for using artificial transcription factor systems.

In certain instances the present disclosure also relates to nucleic acids encoding one or more of the components of the aTF systems described herein, expression vectors (e.g., plasmids, viral vectors, or bacterial vectors) that contain nucleic acids encoding one or more components of the aTF systems described herein, or a host cell that contains such nucleic acids or vectors. Further the present disclosure also relates to pharmaceutical compositions (e.g., for therapeutic or prophylactic use) that contains any of the nucleic acids, vectors, host cells, or the aTF systems (or their components) described herein.

The aTF systems (and nucleic acids and vectors that encode the aTF systems or their components) can have various applications. For example, the aTF systems described herein can be used to modulate (e.g., activate or increase) gene expression, for example to treat various conditions or diseases. For example, the aTF systems described herein can be used to treat sickle cell disease or beta-thalassemia by selectively increasing the expression of the HBG gene expression. The aTF systems described herein can also be used for allele specific activation of endogenous human genes, for example for the treatment of human diseases, e.g., human diseases caused by haploinsufficiency. Further, the aTF systems described herein can be used to identify previously unknown enhancers by assessing whether aTFs that are specific for putative enhancers can modulate the expression of target genes.

### Artificial Transcription Factors

Artificial transcription factors (aTFs) are “designer regulatory proteins comprised of modular units that can be customized to overcome challenges faced by natural [transcription factors] in establishing and maintaining desired cell states.” Heiderscheit et al., “Reprogramming Cell Fate with Artificial Transcription Factors,” FEBS Letters 592:888-900 (2018). aTFs can target cognate sites in the genome through, e.g., a DNA binding domain, and can deliver, e.g., an effector domain to a specific genomic locus, e.g., to activate or repress transcription of targeted genes by recruiting or blocking transcriptional machinery. See id. A number of aTF platforms are known in the art, including, but not limited to clustered regularly interspaced short palindromic repeat-Cas (CRISPR-Cas), (transcription activator-like effectors (TALEs), synthetic molecules, and zinc fingers (ZFs). See id.

The aTFs disclosed herein comprise nucleic acid (e.g., DNA) binding domain(s) (DBDs) and gene expression modulating domain(s) (EMDs).

The nucleic acid sequence binding domain (e.g., an enhancer-binding domain or a promoter-binding domain) can allow the aTFs to be directed to a specific region of a nucleic acid (e.g., genomic DNA).

In some embodiments, the aTF comprises a fusion protein comprising a nucleic acid sequence binding domain or portion thereof, e.g., a catalytically inactive Cas9 or Cpf1, and a gene expression modulating domain, e.g., an activation domain, e.g., p65, VP40, VPR, or p300.

In some embodiments, the gene expression modulating domain is genetically fused to a nucleic acid sequence binding domain or portion thereof, e.g., as a direct fusion aTF.

The nucleic acid sequence binding domains, preferably CRISPR-Cas9 or CRISPR-Cpf1 comprising one or more nuclease-reducing or killing mutation(s), can be fused on the N or C terminus of, e.g., the Cas9 or Cpf1 to a transcriptional activation domain (e.g., a transcriptional activation domain from the VP16 domain form herpes simplex virus (Sadowski et al., 1988, Nature, 335:563-564) or VP64; the p65 domain from the cellular transcription factor NF-kappaB (Ruben et al., 1991, Science, 251:1490-93); a tripartite effector fused to dCas9, composed of activators VP64, p65, and Rta (VPR) linked in tandem, Chavez et al., Nat Methods. 2015 April; 12(4):326-8); or the p300 HAT domain. p300/CBP is a histone acetyltransferase (HAT) whose function is critical for regulating gene expression in mammalian cells. The p300 HAT domain (1284-1673) is catalytically active and can be fused to nucleases for targeted epigenome editing. See Hilton et al., Nat Biotechnol. 2015 May; 33(5):510-7.

In some embodiments, the expression modulating domain is not genetically fused to a nucleic acid sequence binding domain, e.g., as a bi-partite aTF in which the DBD and the regulatory domain are not directly linked but are inducibly brought together (for example, using drug-inducible heterodimerization domains fused to each component).

In some embodiments, the aTF comprises (i) a fusion protein that comprises a nucleic acid sequence binding domain, e.g., a catalytically inactive Cas9 or Cpf1, and a first dimerizing domain, e.g., DmrA(s) and (ii) a fusion protein comprising an expression modulating domain, e.g., an activation domain, e.g., p65, VP40, VPR, or p300, and a second dimerizing domain, e.g., DmrC(s). In some embodiments, the first dimerizing domain and the second dimerizing domain form a heterodimer in the presence of a dimerizing agent, e.g., A/C heterodimerizer.

Any inducible protein dimerizing system can be used, e.g., based on the FK506-binding protein (FKBP), see, e.g., Rollins et al., Proc Natl Acad Sci USA. 2000 Jun. 20; 97(13): 7096-7101; the iDIMERIZE™ Inducible Heterodimer System from Clontech/Takara, wherein the proteins of interest are fused to the DmrA and DmrC binding domains respectively, and dimerization is induced by adding the A/C Heterodimerizer (AP21967). Others are also known, e.g., FKBP with CyP-Fas and FKCsA dimerizing agent (see Belshaw et al., Proceedings of the National Academy of Sciences of the United States of America. 93 (10): 4604-7 (1996)); FKBP and FRB domain of mTOR with Rapamycin dimerizing agent (Rivera et al., Nature Medicine. 2 (9): 1028-32 (1996)); GyrB domain with coumermycin dimerizing agent (Farrar et al., Nature. 383 (6596): 178-81 (1996)); gibberellin-induced dimerization (see Miyamoto et al., Nature Chemical Biology. 8 (5): 465-70 (2012); Miyamoto et al., Nature Chemical Biology. 8 (5): 465-70 (2012)); and protein heterodimerization system based on small molecules cross-linking fusion proteins derived from HaloTags and SNAP-tags (Erhart et al., Chemistry and Biology. 20 (4): 549-57 (2013).

Also provided herein are isolated nucleic acids encoding the fusion proteins, gRNAs, and dimerizing agents; vectors comprising the isolated nucleic acids, optionally operably linked to one or more regulatory domains for expressing the variant proteins, and host cells, e.g., mammalian host cells, comprising the nucleic acids, and optionally expressing the variant proteins.

The aTFs can be codon-optimized for the target organism or cell in which they are expressed.

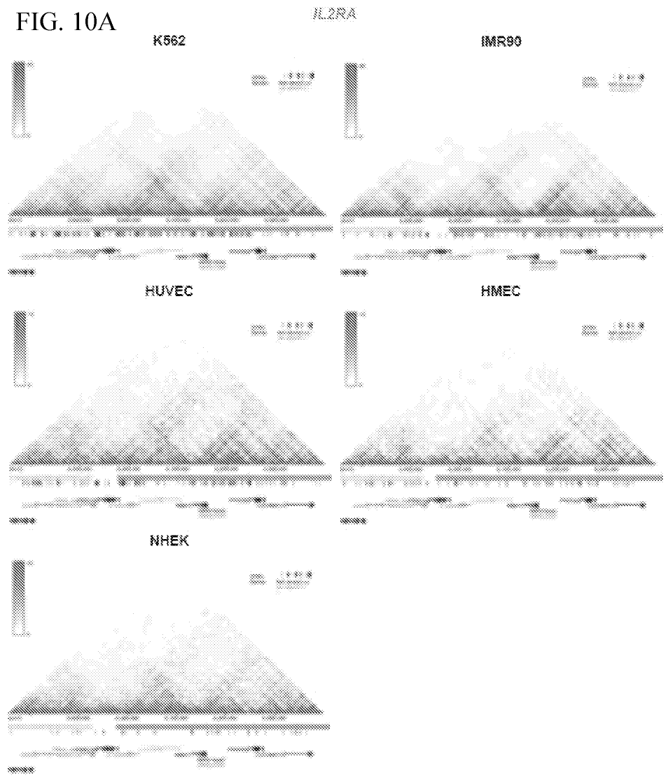

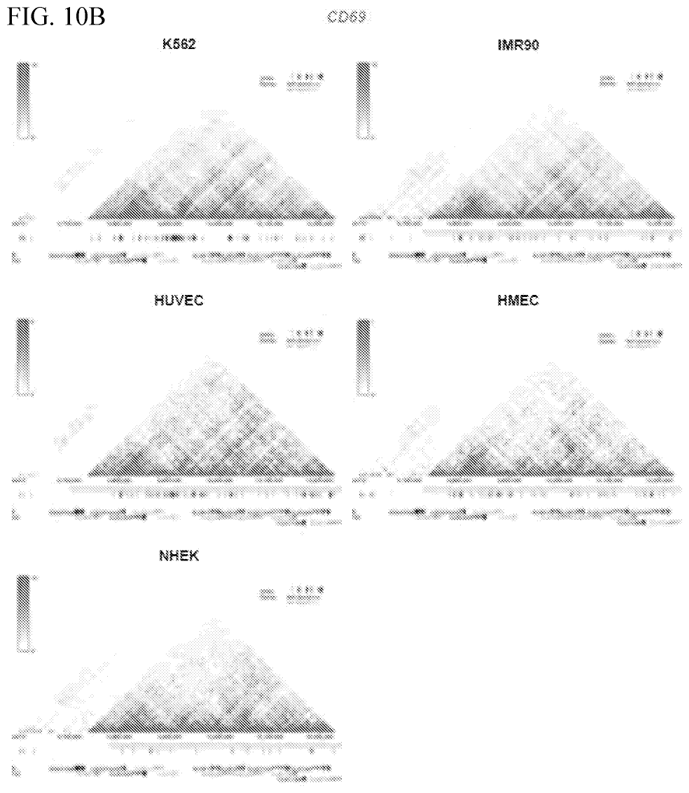

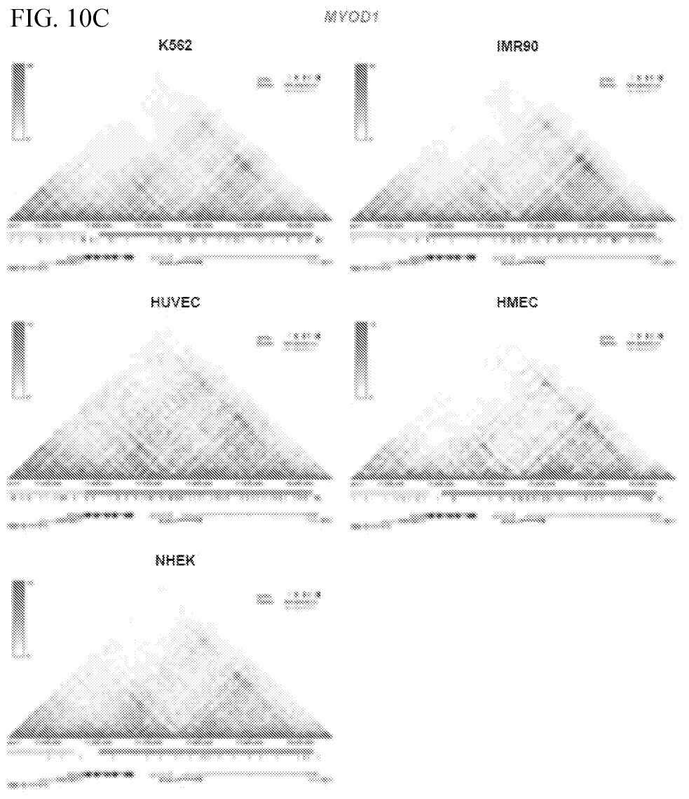

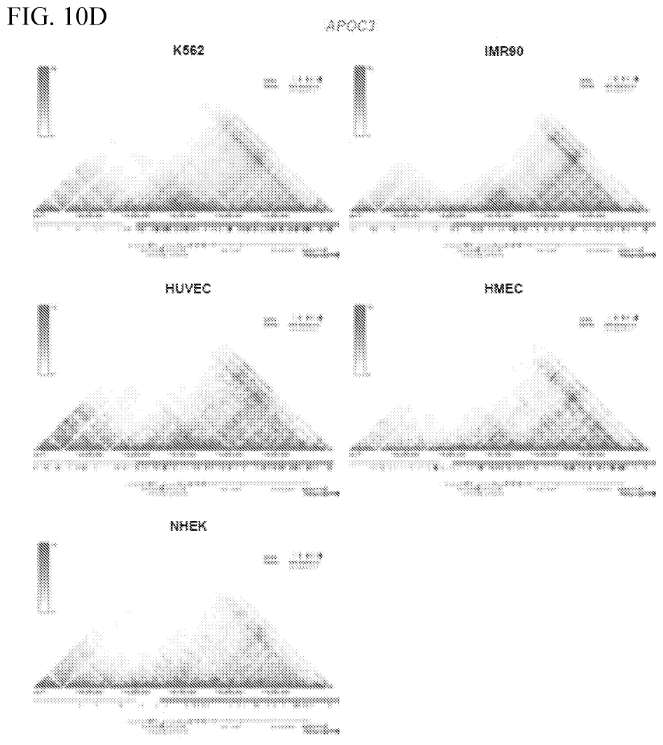

The present disclosure provides a strategy to leverage enhancer sequences to modulate (e.g., upregulate) expression from a target promoter of interest. Doing so requires pre-existing knowledge of an enhancer that interacts with and upregulates a given promoter of interest in at least one cell type. This enhancer sequence can then be activated in other heterotopic cell settings by simply recruiting aTFs to both the enhancer and the target promoter simultaneously. Our finding that we could also activate the APOC3 promoter by directing aTFs to sequences proximal to but outside the boundary of a known enhancer indicates that these types of other enhancer-proximal sequences can also be leveraged to activate a target promoter. The present finding can be used to determine whether three-dimensional proximity between a target promoter and a given potential enhancer-like sequence (e.g., as judged by 3C, 4C, Hi-C or other related assays) might suffice to predict whether simultaneous aTF recruitment to these sites will lead to gene activation. Consistent with this possibility, we found that each of the enhancer-promoter pairs we used in our study lies within a single topologically-associated domain (TAD) across multiple cell types (FIG. 10).

The present disclosure has important implications for our biological understanding of how enhancers normally function. First, the finding that enhancer sequences can be activated heterotopically in multiple cell lines with expression of only aTFs indicate that three-dimensional architectural requirements for such “actions-at-a-distance” between enhancers-promoter pairs are either highly conserved or can be readily induced by aTFs alone if they are both present in the same TAD. In addition, our results distinguish functional characteristics of enhancer sequences versus promoter-proximal sequences for activating transcription when bound by aTFs. Enhancer-bound aTFs appear to be more generally limited to function only as “multipliers” of promoters that are already active. By contrast, aTFs bound to promoter-proximal sequences can turn on an inactive promoter. This difference has important implications for the identification of potential enhancer sequences using aTF (e.g., CRISPRa) screens because an inactive target promoter may not be permissive for identification of an associated enhancer that regulates its activity. Finally, our experiments also improve our understanding of how a single enhancer can dynamically and differentially regulate multiple promoters within a gene cluster. Our results with the beta-globin gene cluster indicates a general mechanism by which enhancers might be re-directed or additionally directed to an alternative target gene simply by upregulating or downregulating different target promoters. This model is consistent with previous studies of hemoglobin gene switching in which LCR activity is re-directed from HBG to HBB in the post-natal stage with both an increased abundance of KLF1 activator observed on the HBB promoter and BCL11A repressor recruited to the HBG promoter (Zhou et al., “Differential binding of erythroid Krupple-like factor to embryonic/fetal globin gene promoters during development,” J Biol Chem 281, 16052-16057, doi:10.1074/jbc.M601182200 (2006); and Liu et al., “Direct Promoter Repression by BCL11A Controls the Fetal to Adult Hemoglobin Switch,” Cell 173, 430-442 e417, doi:10.1016/j.cell.2018.03.016 (2018)); it also indicates that current therapeutic strategies that aim to increase HBG expression for sickle cell disease or beta-thalassemia (Wienert et al., “Wake-up Sleepy Gene: Reactivating Fetal Globin for beta-Hemoglobinopathies. Trends Genet 34, 927-940, doi:10.1016/j.tig.2018.09.004 (2018)) might benefit from repression of the mutant HBB gene promoter.

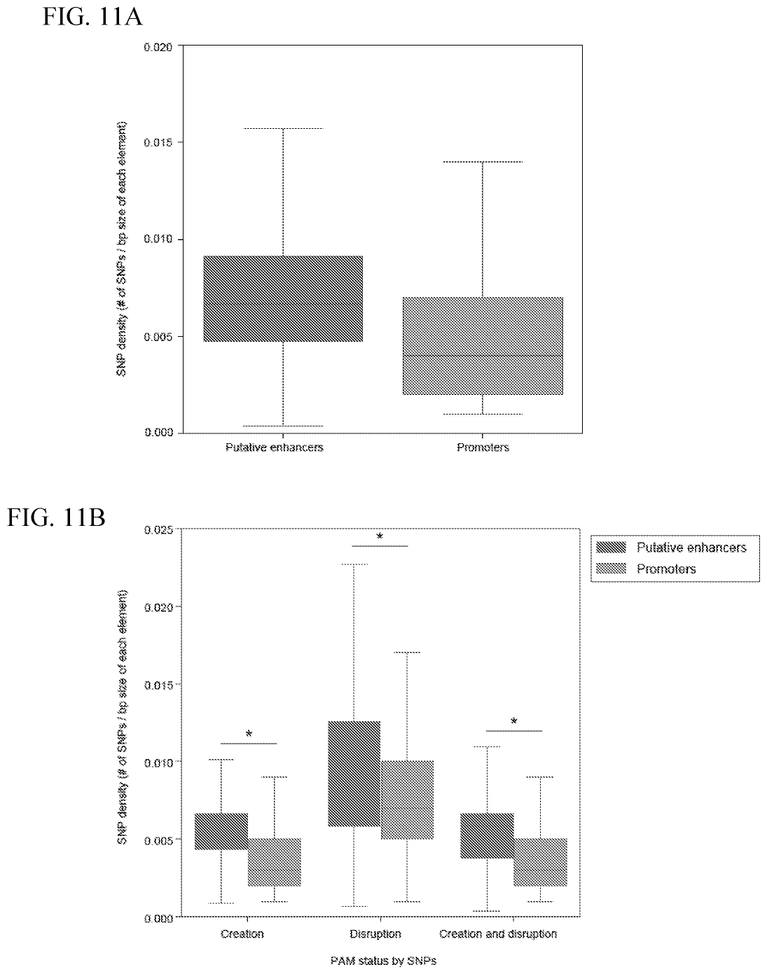

The systems and methods disclosed herein can be leveraged in several ways to provide greater flexibility, optionality, and precision for performing targeted upregulation of human gene expression with aTFs (e.g., CRISPR-based aTFs). We have shown how aTF synergy can be exploited at both a promoter and an enhancer to adjust the dynamic range of gene activation. Given this finding, Cas12a-based aTFs, which have the advantage of being easier to multiplex (Tak et al., “Inducible and multiplex gene regulation using CRISPR-Cpf1-based transcription factors,” Nat Methods 14, 1163-1166, doi:10.1038/nmeth.4483 (2017); and Kleinstiver et al., “Engineered CRISPR-Cas12a variants with increased activities and improved targeting ranges for gene, epigenetic and base editing,” Nat Biotechnol 37, 276-282, doi:10.1038/s41587-018-0011-0 (2019)), can also be used with our strategy to activate enhancer sequences. In addition, our findings demonstrate how this method can be combined with sequence variation in enhancer sequences to achieve allele-selective gene activation. Although allele-specific gene expression induced by native transcription factor binding to regulatory elements has been described in both normal and disease-associated regulation of endogenous genes in human cells (Cavalli et al., “Allele-specific transcription factor binding to common and rare variants associated with disease and gene expression,” Hum Genet 135, 485-497, doi:10.1007/s00439-016-1654-x (2016); Spisak et al., “CAUSEL: an epigenome- and genome-editing pipeline for establishing function of noncoding GWAS variants,” Nat Med, doi:10.1038/nm.3975nm.3975 [pii] (2015); and Bailey et al., “ZNF143 provides sequence specificity to secure chromatin interactions at gene promoters,” Nat Commun 2, 6186, doi:10.1038/ncomms7186 (2015)), our work is, to our knowledge, the first demonstration of how allele-selective gene activation can be accomplished synthetically using aTFs such as CRISPRa. Allele-selective gene activation could provide a general therapeutic strategy for haploinsufficient or dominant-negative diseases (Lek et al., “Analysis of protein-coding genetic variation in 60,706 humans,” Nature 536, 285-291, doi:10.1038/nature19057 (2016); Cooper et al., “Where genotype is not predictive of phenotype: towards an understanding of the molecular basis of reduced penetrance in human inherited disease,” Hum Genet 132, 1077-1130, doi:10.1007/s00439-013-1331-2 (2013); Veitia et al., “Mechanisms of Mendelian dominance,” Clin Genet 93, 419-428, doi:10.1111/cge.13107 (2018); Matharu et al., “CRISPR-mediated activation of a promoter or enhancer rescues obesity caused by haploinsufficiency,” Science 363, doi:10.1126/science.aau0629 (2019); and Dang et al., “Identification of human haploinsufficient genes and their genomic proximity to segmental duplications,” Eur J Hum Genet 16, 1350-1357, doi:10.1038/ejhg.2008.111 (2008)) in which one would preferentially upregulate expression of a wild-type allele over a mutant allele. The ability to use enhancers for allele-selective activation provides an additional and richer source of sequence variation beyond promoters to exploit for this purpose. An analysis of 1000 Genomes Project data we performed found that SNPs that disrupt or create NGG PAM sequences for SpCas9 are greatly enriched genome-wide in putative enhancer sequences compared with promoter sequences: ˜2-fold and ˜12-fold higher for SNP density and for total number of SNPs, respectively (see FIG. 11, and Table 5). Finally, the capability to direct an enhancer to a specific promoter among multiple potential target promoters may enable the generation of more complex spatio-temporal gene expression patterns in heterotopic cell settings. In sum, the enhancer activation strategy described here should broaden the scope and range of both research and therapeutic applications of aTFs (e.g., CRISPR-based aTFs) including more complex library screens to create specific cell phenotypes or functions, synthetic biology strategies to create engineered gene circuits, and epigenetic editing approaches to upregulate a specific gene or allele of interest.

### Nucleic Acid Sequence Binding Domains

In some embodiments, the nucleic acid sequence binding domain is a programmable nucleic acid sequence binding domain such as engineered C2H2 zinc-fingers, transcription activator effector-like effectors (TALEs), and Clustered Regularly Interspaced Short Palindromic Repeats (CRISPR) Cas RNA-guided nucleases (RGNs) and their variants, including catalytically inactive dead Cas9 (dCas9) and its analogs (e.g., as shown in Table 1), and any engineered protospacer-adjacent motif (PAM) or high-fidelity variants (e.g., as shown in Table 2). A programmable nucleic acid sequence binding domain is one that can be engineered to bind to a selected target sequence (e.g., nucleic acid sequences present in enhancers or promoters of target genes).

In some embodiments, the nucleic acid sequence binding domains is specific for a particular promoter or enhancer sequence. In some embodiments, the nucleic acid sequence binding domain is specific for a particular allele of a promoter or enhancer sequence.

CRISPR Based aTFs

Clustered, regularly interspaced, short palindromic repeat (CRISPR) systems encode RNA-guided endonucleases that are essential for bacterial adaptive immunity. See Wright et al., “Biology and Applications of CRISPR Systems: Harnessing Nature's Toolbox for Genome Engineering,” Cell 164, 29-44 (2016). CRISPR-associated (Cas) nucleases can be readily programmed to recognize and cleave target DNA sequences for genome editing in various organisms. Sander et al., “CRISPR-Cas systems for editing, regulating and targeting genomes,” Nat Biotechnol 32, 347-355 (2014); Hsu et al., “Development and applications of CRISPR-Cas9 for genome engineering,” Cell 157, 1262-1278 (2014); Doudna et al., “Genome editing. The new frontier of genome engineering with CRISPR-Cas9,” Science 346, 1258096 (2014); and Maeder et al., “Genome-editing Technologies for Gene and Cell Therapy,” Mol Ther (2016). One class of these nucleases, referred to as Cas9 proteins, complex with two short RNAs: a crRNA and a trans-activating crRNA (tracrRNA). See Jinek et al., “A programmable dual-RNA-guided DNA endonuclease in adaptive bacterial immunity,” Science 337, 816-821 (2012); and Deltcheva et al., “CRISPR RNA maturation by trans-encoded small RNA and host factor RNase III,” Nature 471, 602-607 (2011). The most commonly used Cas9 ortholog, SpCas9, uses a crRNA that has 20 nucleotides (nt) at its 5′ end that are complementary to the “protospacer” region of the target DNA site. Efficient cleavage also requires that SpCas9 recognizes a protospacer adjacent motif (PAM). The crRNA and tracrRNA are usually combined into a single ˜100-nt guide RNA (gRNA) (Jinek et al., “A programmable dual-RNA-guided DNA endonuclease in adaptive bacterial immunity,” Science 337, 816-821 (2012); Cong et al., “Multiplex genome engineering using CRISPR/Cas systems,” Science 339, 819-823 (2013); Mali et al., “RNA-guided human genome engineering via Cas9,” Science 339, 823-826 (2013); and Jinek et al., “RNA-programmed genome editing in human cells,” Elife 2, e00471 (2013)) that directs the DNA cleavage activity of SpCas9. The genome-wide specificities of SpCas9 nucleases paired with different gRNAs have been characterized using many different approaches. See Tsai et al., “GUIDE-seq enables genome-wide profiling of off-target cleavage by CRISPR-Cas nucleases,” Nat Biotechnol 33, 187-197 (2015); Frock et al., “Genome-wide detection of DNA double-stranded breaks induced by engineered nucleases,” Nat Biotechnol 33, 179-186 (2015); Wang et al., “Unbiased detection of off-target cleavage by CRISPR-Cas9 and TALENs using integrase-defective lentiviral vectors,” Nat Biotechnol 33, 175-178 (2015); and Kim et al., “Digenome-seq: genome-wide profiling of CRISPR-Cas9 off-target effects in human cells,” Nat Methods 12, 237-243, 231 p following 243 (2015). SpCas9 variants with substantially improved genome-wide specificities have also been engineered. See Kleinstiver et al., “High-fidelity CRISPR-Cas9 nucleases with no detectable genome-wide off-target effects,” Nature 529, 490-495 (2016); and Slaymaker et al., “Rationally engineered Cas9 nucleases with improved specificity,” Science 351, 84-88 (2016).

Recently, a Cas protein named Cpf1 (also known as Cas12a) has been identified that can also be programmed to cleave target DNA sequences. See Schunder et al., “First indication for a functional CRISPR/Cas system in Francisella tularensis,” Int J Med Microbiol 303, 51-60 (2013); Makarova et al., “An updated evolutionary classification of CRISPR-Cas systems,” Nat Rev Microbiol 13, 722-736 (2015); Zetsche et al., “Cpf1 Is a Single RNA-Guided Endonuclease of a Class 2 CRISPR-Cas System,” Cell 163, 759-771 (2015); and Fagerlund et al., “The Cpf1 CRISPR-Cas protein expands genome-editing tools,” Genome Biol 16, 251 (2015). Unlike SpCas9, Cpf1 requires only a single 42-nt crRNA, which has as many as 23 nt at its 3′ end that are complementary to the protospacer of the target DNA sequence. See Zetsche et al., “Cpf1 Is a Single RNA-Guided Endonuclease of a Class 2 CRISPR-Cas System,” Cell 163, 759-771 (2015). Furthermore, whereas SpCas9 recognizes an NGG PAM sequence that is 3′ of the protospacer, AsCpf1 and LbCp1 recognize TTTN PAMs that are found 5′ of the protospacer. See Zetsche et al., “Cpf1 Is a Single RNA-Guided Endonuclease of a Class 2 CRISPR-Cas System,” Cell 163, 759-771 (2015). Early experiments with AsCpf1 and LbCpf1 showed that these nucleases can be programmed to edit target sites in human cells (see Zetsche et al., “Cpf1 Is a Single RNA-Guided Endonuclease of a Class 2 CRISPR-Cas System,” Cell 163, 759-771 (2015)) but they were tested on only a small number of sites. On-target activities and genome-wide specificities of both AsCpf1 and LbCpf1 were characterized in Kleinstiver & Tsai et al., Nature Biotechnology 2016.

Exemplary CRISPR based aTFs are described herein, and, for example, in WO2018195540A1, which is hereby incorporated by reference in its entirety.

Although herein we refer to Cas9, in general any Cas9-like protein could be used (including the related Cpf1/Cas12a enzyme classes), for example, those listed in Table 1, unless specifically indicated.

TABLE 1
List of Exemplary Cas9 or Cas12a Orthologs

| | UniProt or GenBank | Nickase Mutations/ |
|---|---|---|
| Ortholog | Accession Number | Catalytic residues |
| S. pyogenes Cas9 | Q99ZW2.1 | D10A, E762A, H840A, |
| (SpCas9) | | N854A, N863A, |
| | | D986A¹⁷ |
| S. aureus Cas9 (SaCas9) | J7RUA5.1 | D10A and N580¹⁸ |
| S. thermophilus Cas9 | G3ECR1.2 | D31A and N891A¹⁹ |
| (St1Cas9) | | |
| S. pasteurianus Cas9 | BAK30384.1 | D10, H599* |
| (SpaCas9) | | |
| C. jejuni Cas9 (CjCas9) | Q0P897.1 | D8A, H559A²⁰ |
| F. novicida Cas9 | A0Q5Y3.1 | D11, N995²¹ |
| (FnCas9) | | |
| P. lavamentivorans Cas9 | A7HP89.1 | D8, H601* |
| (PlCas9) | | |
| C. lari Cas9 (ClCas9) | G1UFN3.1 | D7, H567* |
| Pasteurella multocida | Q9CLT2.1 | |
| Cas9 | | |
| F. novicida Cpf1 | A0Q7Q2.1 | D917, E1006, D1255²¹ |
| (FnCpf1) | | |
| M. bovoculi Cpf1 | WP_052585281.1 | D986A** |
| (MbCpf1) | | |
| A. sp. BV3L6 Cpf1 | U2UMQ6.1 | D908, 993E, Q1226, |
| (AsCpf1) | | D1263²³ |
| L. bacterium N2006 | A0A182DWE3.1 | D832A²⁴ |
| (LbCpf1) | | |

*predicted based on UniRule annotation on the UniProt database.
**Unpublished but deposited at addgene by Ervin Welker: pTE4565 (Addgene plasmid # 88903)
These orthologs, and mutants and variants thereof as known in the art, can be used in any of the fusion proteins described herein. See, e.g., WO 2017/040348 (which describes variants of SaCas9 and SpCas 9 with increased specificity) and WO 2016/141224 (which describes variants of SaCas9 and SpCas 9 with altered PAM specificity).

TABLE 2
List of Exemplary High Fidelity and/or PAM-relaxed RGN Orthologs

| Published | | |
|---|---|---|
| HF/PAM-RGN | | |
| variants | PMID | Mutations* |
| S. pyogenes Cas9 | 26628643 | K810A/K1003A/R1060A (1.0); |
| (SpCas9) | | K848A/K1003A/R1060A(1.1) |
| eSpCas9 | | |
| S. pyogenes Cas9 | 29431739 | M495V/Y515N/K526E/R661Q; |
| (SpCas9) | | (M495V/Y515N/K526E/R661S; |
| evoCas9 | | M495V/Y515N/K526E/R661L) |
| S. pyogenes Cas9 | 26735016 | N497A/R661A/Q695A/Q926A |
| (SpCas9) HF1 | | |
| S. pyogenes Cas9 | 30082871 | R691A |
| (SpCas9) HiFi | | |
| Cas9 | | |
| S. pyogenes Cas9 | 28931002 | N692A, M694A, Q695A, H698A |
| (SpCas9) | | |
| HypaCas9 | | |
| S. pyogenes Cas9 | 30082838 | F539S, M763I, K890N |
| (SpCas9) Sniper- | | |
| Cas9 | | |
| S. pyogenes Cas9 | 29512652 | A262T, R324L, S409I, E480K, E543D, M694I, |
| (SpCas9) xCas9 | | E1219V |
| S. pyogenes Cas9 | 30166441 | R1335V, L1111R, D1135V, G1218R, |
| (SpCas9) | | E1219F, A1322R, T1337R |
| SpCas9-NG | | |
| S. pyogenes Cas9 | 26098369 | D1135V, R1335Q, T1337R; |
| (SpCas9) | | D1135V/G1218R/R1335E/T1337R |
| VQR/VRER | | |
| S. aureus Cas9 | 26524662 | E782K/N968K/R1015H |
| (SaCas9)-KKH | | |
| enAsCas12a | USSN 15/960,271 | One or more of: E174R, S170R, S542R, K548R, |
| | | K548V, N551R, N552R, K607R, K607H, e.g., |
| | | E174R/S542R/K548R, E174R/S542R/K607R, |
| | | E174R/S542R/K548V/N552R, |
| | | S170R/S542R/K548R, S170R/E174R, |
| | | E174R/S542R, S170R/S542R, |
| | | E174R/S542R/K548R/N551R, |
| | | E174R/S542R/K607H, S170R/S542R/K607R, or |
| | | S170R/S542R/K548V/N552R |
| enAsCas12a-HF | USSN 15/960,271 | One or more of: E174R, S542R, K548R, e.g., |
| | | E174R/S542R/K548R, E174R/S542R/K607R, |
| | | E174R/S542R/K548V/N552R, |
| | | S170R/S542R/K548R, S170R/E174R, |
| | | E174R/S542R, S170R/S542R, |
| | | E174R/S542R/K548R/N551R, |
| | | E174R/S542R/K607H, S170R/S542R/K607R, or |
| | | S170R/S542R/K548V/N552R, with the addition of |
| | | one or more of: N282A, T315A, N515A and K949A |
| enLbCas12a(HF) | USSN 15/960,271 | One or more of T152R, T152K, D156R, D156K, |
| | | Q529K, G532R, G532K, G532Q, K538R, K538V, |
| | | D541R, Y542R, M592A, K595R, K595H, K595S or |
| | | K595Q, e.g., D156R/G532R/K538R, |
| | | D156R/G532R/K595R, |
| | | D156R/G532R/K538V/Y542R, |
| | | T152R/G532R/K538R, T152R/D156R, |
| | | D156R/G532R, T152R/G532R, |
| | | D156R/G532R/K538R/D541R, |
| | | D156R/G532R/K595H, T152R/G532R/K595R, |
| | | T152R/G532R/K538V/Y542R, optionally with the |
| | | addition of one or more of: N260A, N256A, K514A, |
| | | D505A, K881A, S286A, K272A, K897A |
| enFnCas12a(HF) | USSN 15/960,271 | One or more of T177A, K180R, K180K, E184R, |
| | | E184K, T604K, N607R, N607K, N607Q, K613R, |
| | | K613V, D616R, N617R, M668A, K671R, K671H, |
| | | K671S, or K671Q, e.g., E184R/N607R/K613R, |
| | | E184R/N607R/K671R, |
| | | E184R/N607R/K613V/N617R, |
| | | K180R/N607R/K613R, K180R/E184R, |
| | | E184R/N607R, K180R/N607R, |
| | | E184R/N607R/K613R/D616R, |
| | | E184R/N607R/K671H, K180R/N607R/K671R, |
| | | K180R/N607R/K613V/N617R, optionally with the |
| | | addition of one or more of: N305A, N301A, K589A, |
| | | N580A, K962A, S334A, K320A, K978A |
| S. pyogenes Cas9 | 32217751 | D1135L, S1136W, G1218K, E1219Q, R1335Q, |
| (SpGas9) SpG | | T1337R |
| S. pyogenes Cas9 | 32217751 | A61R, L1111R, D1135L, S1136W, G1218K, |
| (SpGas9) SpRY | | E1219Q, N1317R, A1322R, R1333P, R1335Q, |
| | | T1337R |

*predicted based on UniRule annotation on the UniProt database.

The Cas9 nuclease from S. pyogenes (hereafter spCas9) can be guided via simple base pair complementarity between 17-20 nucleotides of an engineered guide RNA (gRNA), e.g., a single guide RNA or crRNA/tracrRNA pair, and the complementary strand of a target genomic DNA sequence of interest that lies next to a protospacer adjacent motif (PAM), e.g., a PAM matching the sequence NGG or NAG (Shen et al., Cell Res (2013); Dicarlo et al., Nucleic Acids Res (2013); Jiang et al., Nat Biotechnol 31, 233-239 (2013); Jinek et al., Elife 2, e00471 (2013); Hwang et al., Nat Biotechnol 31, 227-229 (2013); Cong et al., Science 339, 819-823 (2013); Mali et al., Science 339, 823-826 (2013c); Cho et al., Nat Biotechnol 31, 230-232 (2013); Jinek et al., Science 337, 816-821 (2012)). The engineered CRISPR from Prevotella and Francisella 1 (Cpf1, also known as Cas12a) nuclease can also be used, e.g., as described in Zetsche et al., Cell 163, 759-771 (2015); Schunder et al., Int J Med Microbiol 303, 51-60 (2013); Makarova et al., Nat Rev Microbiol 13, 722-736 (2015); Fagerlund et al., Genome Biol 16, 251 (2015). Unlike SpCas9, Cpf1/Cas12a requires only a single 42-nt crRNA, which has 23 nt at its 3′ end that are complementary to the protospacer of the target DNA sequence (Zetsche et al., 2015). Furthermore, whereas SpCas9 recognizes an NGG PAM sequence that is 3′ of the protospacer, AsCpf1 and LbCp1 recognize TTTN PAMs that are found 5′ of the protospacer (Id.). The wild-type sequence of spCas9 (SEQ ID NO:1) is as follows:

MDKKYSIGLDIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLELAAKNLSDAILLSDILRVNTEITKA
PLSASMIKRYDEHHQDLTLLKALVRQQLPEKYKEI
FFDQSKNGYAGYIDGGASQEEFYKFIKPILEKMDG
TEELLVKLNREDLLRKQRTFDNGSIPHQIHLGELH
AILRRQEDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLFDDKVMKQLKRRRYTGWGRLSRK
LINGIRDKQSGKTILDFLKSDGFANRNFMQLIHDD
SLTFKEDIQKAQVSGQGDSLHEHIANLAGSPAIKK
GILQTVKVVDELVKVMGRHKPENIVIEMARENQTT
QKGQKNSRERMKRIEEGIKELGSQILKEHPVENTQ
LQNEKLYLYYLQNGRDMYVDQELDINRLSDYDVDH
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTEI
TLANGEIRKRPLIETNGETGEIVWDKGRDFATVRK
VLSMPQVNIVKKTEVQTGGFSKESILPKRNSDKLI
ARKKDWDPKKYGGFDSPTVAYSVLVVAKVEKGKSK
KLKSVKELLGITIMERSSFEKNPIDFLEAKGYKEV
KKDLIIKLPKYSLFELENGRKRMLASAGELQKGNE
LALPSKYVNFLYLASHYEKLKGSPEDNEQKQLFVE
QHKHYLDEIIEQISEFSKRVILADANLDKVLSAYN
KHRDKPIREQAENIIHLFTLTNLGAPAAFKYFDTT
IDRKRYTSTKEVLDATLIHQSITGLYETRIDLSQL
GGD

Wild-type spCas9 has 2 endonuclease domains. The discontinuous RuvC-like domain (approximately residues 1-62,718-765 and 925-1102) recognizes and cleaves the target DNA noncomplementary to crRNA while the HNH nuclease domain (residues 810-872) cleaves the target DNA complementary to crRNA. See Jinek et al., “A Programmable Dual-RNA-Guided DNA Endonuclease in Adaptive Bacterial Immunity,” Science 337:816-21 (2012) and Nishimasu et al., “Crystal Structure of Cas9 in Complex with Guide RNA and Target DNA,” Cell 156:935-49 (2014).

Wild-type spCas9 has a bilobed architecture with a recognition lobe (REC, residues 60-718) and a discontinuous nuclease lobe (NUC, residues 1-59 and 719-1368). See Nishimasu et al., “Crystal Structure of Cas9 in Complex with Guide RNA and Target DNA,” Cell 156:935-49 (2014); Jiang et al., “A Cas9-Guide RNA Complex Preorganized for Target DNA Recognition,” Science 348:1477-81 (2015); and Jinek et al., “Structures of Cas9 endonucleases reveal RNA-mediated conformational activation,” Science 343:154997 (2014). The crRNA-target DNA lies in a channel between the 2 lobes (See Nishimasu et al., “Crystal Structure of Cas9 in Complex with Guide RNA and Target DNA,” Cell 156:935-49 (2014); Jiang et al., “A Cas9-Guide RNA Complex Preorganized for Target DNA Recognition,” Science 348:1477-81 (2015); and and Jiang et al, “Structures of a CRISPR_Cas9 R-loop Complex Primed for DNA Cleavage,” Science 351:867-71 (2016)). Binding of sgRNA induces large conformational changes further enhanced by target DNA binding (see Jiang et al., “STRUCTURAL BIOLOGY. A Cas9-guide RNA Complex Preorganized for Target DNA Recognition,” Science 348:1477-81 (2015); and Jiang et al, “Structures of a CRISPR_Cas9 R-loop Complex Primed for DNA Cleavage,” Science 351:867-71 (2016)). REC recognizes and binds differing regions of an artificial sgRNA in a sequence-independent manner. Deletions of parts of this lobe abolish nuclease activity (See Nishimasu et al., “Crystal Structure of Cas9 in Complex with Guide RNA and Target DNA,” Cell 156:935-49 (2014)).

The PAM-interacting domain of wild-type spCas9 (PI domain, approximately residues 1099-1368) recognizes the PAM motif; swapping the PI domain of this enzyme with that from S. thermophilus St3Cas9 (AC Q03JI6) prevents cleavage of DNA with the endogenous PAM site (5′-NGG-3′) but confers the ability to cleave DNA with the PAM site specific for St3 CRISPRs. See Nishimasu et al., “Crystal Structure of Cas9 in Complex with Guide RNA and Target DNA,” Cell 156:935-49 (2014).

In some embodiments, the present system utilizes a wild type or variant Cas9 protein from S. pyogenes or Staphylococcus aureus, or a wild type or variant Cpf1 protein from Acidaminococcus sp. BV3L6 or Lachnospiraceae bacterium ND2006 either as encoded in bacteria or codon-optimized for expression in mammalian cells and/or modified in its PAM recognition specificity and/or its genome-wide specificity. A number of variants have been described; see, e.g., WO 2016/141224, PCT/US2016/049147, Kleinstiver et al., Nat Biotechnol. 2016 August; 34(8):869-74; Tsai and Joung, Nat Rev Genet. 2016 May; 17(5):300-12; Kleinstiver et al., Nature. 2016 Jan. 28; 529(7587):490-5; Shmakov et al., Mol Cell. 2015 Nov. 5; 60(3):385-97; Kleinstiver et al., Nat Biotechnol. 2015 December; 33(12):1293-1298; Dahlman et al., Nat Biotechnol. 2015 November; 33(11):1159-61; Kleinstiver et al., Nature. 2015 Jul. 23; 523(7561):481-5; Wyvekens et al., Hum Gene Ther. 2015 July; 26(7):425-31; Hwang et al., Methods Mol Biol. 2015; 1311:317-34; Osborn et al., Hum Gene Ther. 2015 February; 26(2):114-26; Konermann et al., Nature. 2015 Jan. 29; 517(7536):583-8; Fu et al., Methods Enzymol. 2014; 546:21-45; and Tsai et al., Nat Biotechnol. 2014 June; 32(6):569-76, inter alia. Concerning rAPOBEC1 itself, a number of variants have been described, e.g. Chen et al, RNA. 2010 May; 16(5):1040-52; Chester et al, EMBO J. 2003 Aug. 1; 22(15):3971-82; Teng et al, J Lipid Res. 1999 April; 40(4):623-35.; Navaratnam et al, Cell. 1995 Apr. 21; 81(2):187-95; MacGinnitie et al, J Biol Chem. 1995 Jun. 16; 270(24):14768-75; Yamanaka et al, J Biol Chem. 1994 Aug. 26; 269(34):21725-34. The guide RNA is expressed or present in the cell together with the Cas9 or Cpf1. Either the guide RNA or the nuclease, or both, can be expressed transiently or stably in the cell or introduced as a purified protein or nucleic acid.

In some embodiments, the Cas9 also includes one of the following mutations, which reduce nuclease activity of the Cas9; e.g., for SpCas9, mutations at D10 (e.g., D10A) or H840 (e.g., H840A) (which creates a single-strand nickase).

In some embodiments, the SpCas9 variants also include mutations at one of each of the two sets of the following amino acid positions, which together destroy the nuclease activity of the Cas9: D10, E762, D839, H983, or D986 and H840 or N863, e.g., D10A/D10N and H840A/H840N/H840Y, to render the nuclease portion of the protein catalytically inactive; substitutions at these positions could be alanine (as they are in Nishimasu al., Cell 156, 935-949 (2014)), or other residues, e.g., glutamine, asparagine, tyrosine, serine, or aspartate, e.g., E762Q, H983N, H983Y, D986N, N863D, N863S, or N863H (see WO 2014/152432).

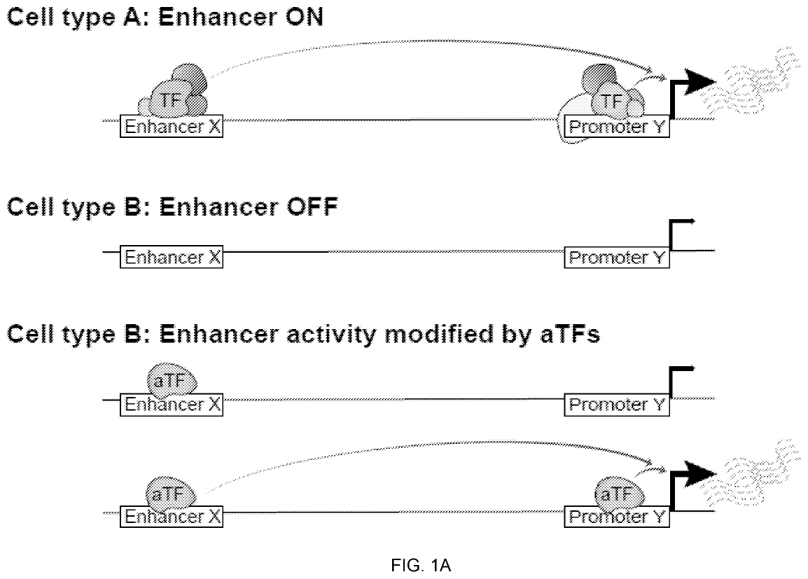

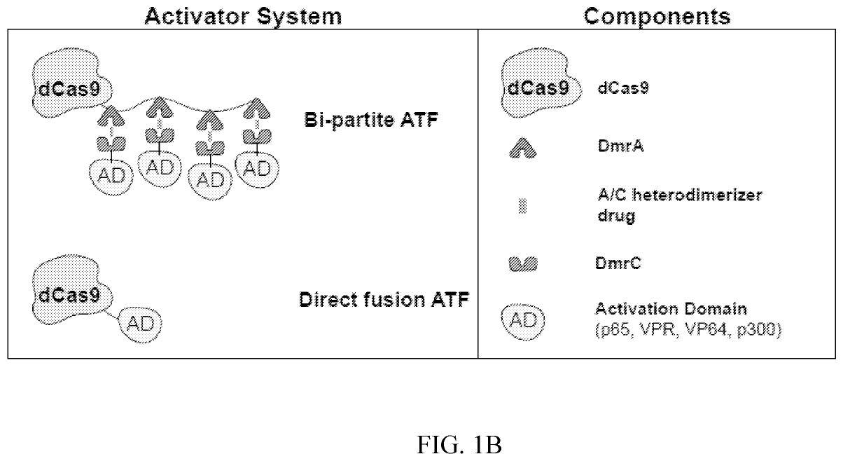

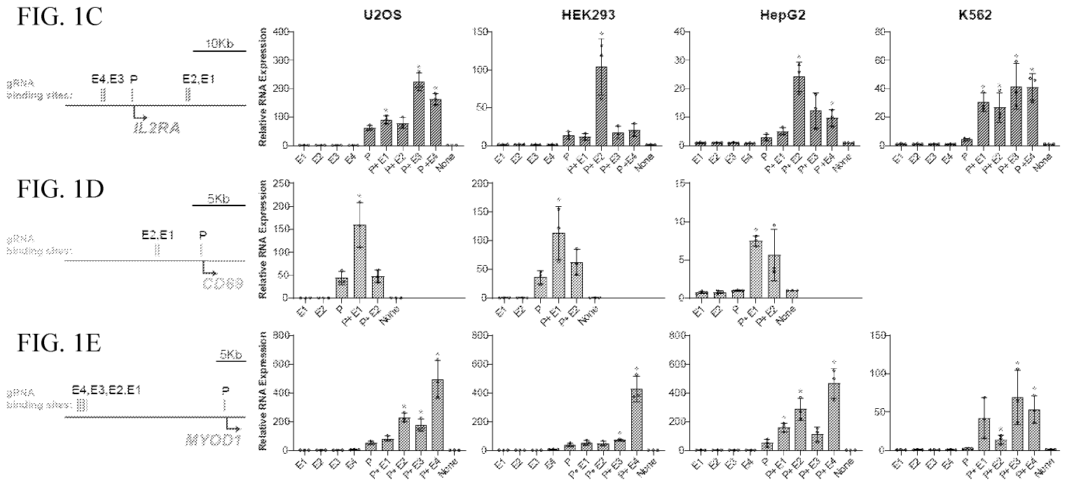

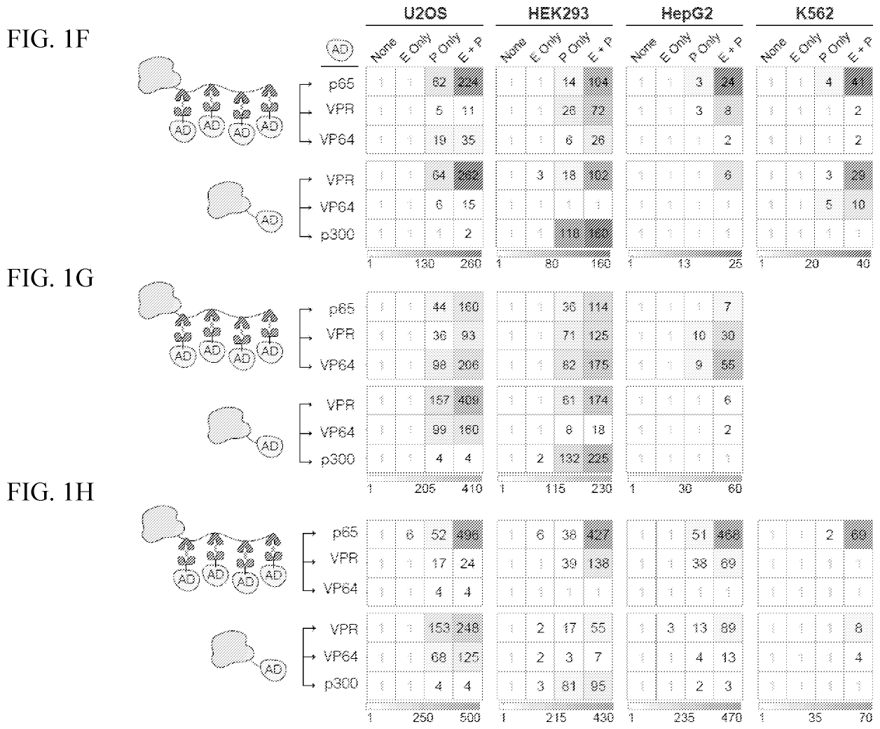

Cas9 molecules of a variety of species can be used in the methods and compositions described herein. While the S. pyogenes and S. thermophilus Cas9 molecules are the subject of much of the disclosure herein, Cas9 molecules of, derived from, or based on the Cas9 proteins of other species listed herein can be used as well. In other words, while the much of the description herein uses S. pyogenes and S. thermophilus Cas9 molecules, Cas9 molecules from the other species can replace them. Such species include those set forth in the following table, which was created based on supplementary FIG. 1 of Chylinski et al., 2013.

Alternative Cas9 proteins

| GenBank Acc No. | Bacterium |
|---|---|
| 303229466 | Veillonella atypica ACS-134-V-Col7a |
| 34762592 | Fusobacterium nucleatum subsp. vincentii |
| 374307738 | Filifactor alocis ATCC 35896 |
| 320528778 | Solobacterium moorei F0204 |
| 291520705 | Coprococcus catus GD-7 |
| 42525843 | Treponema denticola ATCC 35405 |
| 304438954 | Peptoniphilus duerdenii ATCC BAA-1640 |
| 224543312 | Catenibacterium mitsuokai DSM 15897 |
| 24379809 | Streptococcus mutans UA159 |
| 15675041 | Streptococcus pyogenes SF370 |
| 16801805 | Listeria innocua Clip11262 |
| 116628213 | Streptococcus thermophilus LMD-9 |
| 323463801 | Staphylococcus pseudintermedius ED99 |
| 352684361 | Acidaminococcus intestini RyC-MR95 |
| 302336020 | Olsenella uli DSM 7084 |
| 366983953 | Oenococcus kitaharae DSM 17330 |
| 310286728 | Bifidobacterium bifidum S17 |
| 258509199 | Lactobacillus rhamnosus GG |
| 300361537 | Lactobacillus gasseri JV-V03 |
| 169823755 | Finegoldia magna ATCC 29328 |
| 47458868 | Mycoplasma mobile 163K |
| 284931710 | Mycoplasma gallisepticum str. F |
| 363542550 | Mycoplasma ovipneumoniae SC01 |
| 384393286 | Mycoplasma canis PG 14 |
| 71894592 | Mycoplasma synoviae 53 |
| 238924075 | Eubacterium rectale ATCC 33656 |
| 116627542 | Streptococcus thermophilus LMD-9 |
| 315149830 | Enterococcus faecalis TX0012 |
| 315659848 | Staphylococcus lugdunensis M23590 |
| 160915782 | Eubacterium dolichum DSM 3991 |
| 336393381 | Lactobacillus coryniformis subsp. torquens |
| 310780384 | Ilyobacter polytropus DSM 2926 |
| 325677756 | Ruminococcus albus 8 |
| 187736489 | Akkermansia muciniphila ATCC BAA-835 |
| 117929158 | Acidothermus cellulolyticus 11B |
| 189440764 | Bifidobacterium longum DJO10A |
| 283456135 | Bifidobacterium dentium Bd1 |
| 38232678 | Corynebacterium diphtheriae NCTC 13129 |
| 187250660 | Elusimicrobium minutum Pei191 |
| 319957206 | Nitratifractor salsuginis DSM 16511 |
| 325972003 | Sphaerochaeta globus str. Buddy |
| 261414553 | Fibrobacter succinogenes subsp. succinogenes |
| 60683389 | Bacteroides fragilis NCTC 9343 |
| 256819408 | Capnocytophaga ochracea DSM 7271 |
| 90425961 | Rhodopseudomonas palustris BisB18 |
| 373501184 | Prevotella micans F0438 |
| 294674019 | Prevotella ruminicola 23 |
| 365959402 | Flavobacterium columnare ATCC 49512 |
| 312879015 | Aminomonas paucivorans DSM 12260 |
| 83591793 | Rhodospirillum rubrum ATCC 11170 |
| 294086111 | Candidatus Puniceispirillum marinum IMCC1322 |
| 121608211 | Verminephrobacter eiseniae EF01-2 |
| 344171927 | Ralstonia syzygii R24 |
| 159042956 | Dinoroseobacter shibae DFL 12 |
| 288957741 | Azospirillum sp- B510 |
| 92109262 | Nitrobacter hamburgensis X14 |
| 148255343 | Bradyrhizobium sp- BTAi1 |
| 34557790 | Wolinella succinogenes DSM 1740 |
| 218563121 | Campylobacter jejuni subsp. jejuni |
| 291276265 | Helicobacter mustelae 12198 |
| 229113166 | Bacillus cereus Rock1-15 |
| 222109285 | Acidovorax ebreus TPSY |
| 189485225 | uncultured Termite group 1 |
| 182624245 | Clostridium perfringens D str. |
| 220930482 | Clostridium cellulolyticum H10 |
| 154250555 | Parvibaculum lavamentivorans DS-1 |
| 257413184 | Roseburia intestinalis L1-82 |
| 218767588 | Neisseria meningitidis Z2491 |
| 15602992 | Pasteurella multocida subsp. multocida |
| 319941583 | Sutterella wadsworthensis 3 1 |
| 254447899 | gamma proteobacterium HTCC5015 |
| 54296138 | Legionella pneumophila str. Paris |
| 331001027 | Parasutterella excrementihominis YIT 11859 |
| 34557932 | Wolinella succinogenes DSM 1740 |
| 118497352 | Francisella novicida U112 |

The constructs and methods described herein can include the use of any of those Cas9 proteins, and their corresponding guide RNAs or other guide RNAs that are compatible. The Cas9 from Streptococcus thermophilus LMD-9 CRISPR1 system has been shown to function in human cells in Cong et al (Science 339, 819 (2013)). Additionally, Jinek et al. showed in vitro that Cas9 orthologs from S. thermophilus and L. innocua, (but not from N. meningitidis or C. jejuni, which likely use a different guide RNA), can be guided by a dual S. pyogenes gRNA to cleave target plasmid DNA, albeit with slightly decreased efficiency.

In some embodiments, the present system utilizes the Cas9 protein from S. pyogenes, either as encoded in bacteria or codon-optimized for expression in mammalian cells, containing mutations at D10, E762, H983, or D986 and H840 or N863, e.g., D10A/D10N and H840A/H840N/H840Y, to render the nuclease portion of the protein catalytically inactive; substitutions at these positions could be alanine (as they are in Nishimasu al., Cell 156, 935-949 (2014)) or they could be other residues, e.g., glutamine, asparagine, tyrosine, serine, or aspartate, e.g., E762Q, H983N, H983Y, D986N, N863D, N863S, or N863H. The sequence of the catalytically inactive S. pyogenes Cas9 that can be used in the methods and compositions described herein is as follows; the exemplary mutations of D10A and H840A are in bold and underlined.

```
(SEQ ID NO: 228)
                10 20 30
MDKKYSIGLA IGTNSVGWAV ITDEYKVPSK
                40 50 60
KFKVLGNTDR HSIKKNLIGA LLFDSGETAE
                70 80 90
ATRLKRTARR RYTRRKNRIC YLQEIFSNEM
              100 110 120
AKVDDSFFHR LEESFLVEED KKHERHPIFG
              130 140 150
NIVDEVAYHE KYPTIYHLRK KLVDSTDKAD
              160 170 180
LRLIYLALAH MIKFRGHFLI EGDLNPDNSD
              190 200 210
VDKLFIQLVQ TYNQLFEENP INASGVDAKA
              220 230 240
ILSARLSKSR RLENLIAQLP GEKKNGLFGN
              250 260 270
LIALSLGLTP NFKSNFDLAE DAKLQLSKDT
              280 290 300
YDDDLDNLLA QIGDQYADLF LAAKNLSDAI
              310 320 330
LLSDILRVNT EITKAPLSAS MIKRYDEHHQ
              340 350 360
DLTLLKALVR QQLPEKYKEI FFDQSKNGYA
              370 380 390
GYIDGGASQE EFYKFIKPIL EKMDGTEELL
              400 410 420
VKLNREDLLR KQRTFDNGSI PHQIHLGELH
              430 440 450
AILRRQEDFY PFLKDNREKI EKILTFRIPY
              460 470 480
YVGPLARGNS RFAWMTRKSE ETITPWNFEE
              490 500 510
VVDKGASAQS FIERMTNFDK NLPNEKVLPK
              520 530 540
HSLLYEYFTV YNELTKVKYV TEGMRKPAFL
              550 560 570
SGEQKKAIVD LLFKTNRKVT VKQLKEDYFK
              580 590 600
KIECFDSVEI SGVEDRFNAS LGTYHDLLKI
              610 620 630
IKDKDFLDNE ENEDILEDIV LTLTLFEDRE
              640 650 660
MIEERLKTYA HLFDDKVMKQ LKRRRYTGWG
              670 680 690
RLSRKLINGI RDKQSGKTIL DFLKSDGFAN
              700 710 720
RNFMQLIHDD SLTFKEDIQK AQVSGQGDSL
              730 740 750
HEHIANLAGS PAIKKGILQT VKVVDELVKV
              760 770 780
MGRHKPENIV IEMARENQTT QKGQKNSRER
              790 800 810
MKRIEEGIKE LGSQILKEHP VENTQLQNEK
              820 830 840
LYLYYLQNGR DMYVDQELDI NRLSDYDVDA
              850 860 870
IVPQSFLKDD SIDNKVLTRS DKNRGKSDNV
              880 890 900
PSEEWKKMK NYVVRQLLNAK LITQRKFDNL
              910 920 930
TKAERGGLSE LDKAGFIKRQ LVETRQITKH
              940 950 960
VAQILDSRMN TKYDENDKLI REVKVITLKS
              970 980 990
KLVSDFRKDF QFYKVREINN YHHAHDAYLN
            1000 1010 1020
AVVGTALIKK YPKLESEFVY GDYKVYDVRK
            1030 1040 1050
MIAKSEQEIG KATAKYFFYS NIMNFFKTEI
            1060 1070 1080
TLANGEIRKR PLIETNGETG EIVWDKGRDF
            1090 1100 1110
ATVRKVLSMP QVNIVKKTEV QTGGFSKESI
            1120 1130 1140
LPKRNSDKLI ARKKDWDPKK YGGFDSPTVA
            1150 1160 1170
YSVLVVAKVE KGKSKKLKSV KELLGITIME
            1180 1190 1200
RSSFEKNPID FLEAKGYKEV KKDLIIKLPK
            1210 1220 1230
YSLFELENGR KRMLASAGEL QKGNELALPS
            1240 1250 1260
KYVNFLYLAS HYEKLKGSPE DNEQKQLFVE
            1270 1280 1290
QHKHYLDEII EQISEFSKRV ILADANLDKV
            1300 1310 1320
LSAYNKHRDK PIREQAENII HLFTLTNLGA
            1330 1340 1350
PAAFKYFDTT IDRKRYTSTK EVLDATLIHQ
            1360
SITGLYETRI DLSQLGGD
```

In some embodiments, the Cas9 nuclease used herein is at least about 50% identical to the sequence of S. pyogenes Cas9, i.e., at least 50% identical to SEQ ID NO:13. In some embodiments, the nucleotide sequences are about 50%, 55%, 60%, 65%, 70%, 75%, 80%, 85%, 90%, 95%, 99% or 100% identical to SEQ ID NO:228.

In some embodiments, the catalytically inactive Cas9 used herein is at least about 50% identical to the sequence of the catalytically inactive S. pyogenes Cas9, i.e., at least 50%, 55%, 60%, 65%, 70%, 75%, 80%, 85%, 90%, 95%, 99% or 100% identical to SEQ ID NO:228, wherein the mutations at D10 and H840, e.g., D10A/D10N and H840A/H840N/H840Y are maintained.

In some embodiments, any differences from SEQ ID NO:228 are in non-conserved regions, as identified by sequence alignment of sequences set forth in Chylinski et al., RNA Biology 10:5, 1-12; 2013 (e.g., in supplementary FIG. 1 and supplementary table 1 thereof); Esvelt et al., Nat Methods. 2013 November; 10(11):1116-21 and Fonfara et al., Nucl. Acids Res. (2014) 42 (4): 2577-2590. [Epub ahead of print 2013 Nov. 22] doi:10.1093/nar/gkt1074, and wherein the mutations at D10 and H840, e.g., D10A/D10N and H840A/H840N/H840Y are maintained.

In some embodiments, the nucleic acid sequence binding domain comprises a Cpf1 protein, e.g., LbCpf1. The LbCpf1 wild type protein sequence is as follows:
LbCpf1—Type V CRISPR-associated protein Cpf1 [Lachnospiraceae bacterium ND2006], GenBank Acc No. WP_051666128.1 (SEQ ID NO:3)

| | |
|---|---|
| 1 | 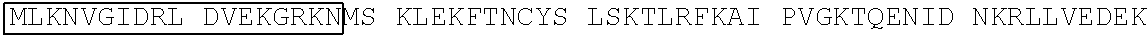 |
| 61 | RAEDYKGVKK LLDRYYLSFI NDVLHSIKLK NLNNYISLFR KKTRTEKENK ELENLEINLR |
| 121 | KEIAKAFKGN EGYKSLFKKD IIETILPEFL DDKDEIALVN SFNGFTTAFT GFFDNRENMF |
| 181 | SEEAKSTSIA FRCINENLTR YISNMDIFEK VDAIFDKHEV QEIKEKILNS DYDVEDFFEG |
| 241 | EFFNFVLTQE GIDVYNAIIG GFVTESGEKI KGLNEYINLY NQKTKQKLPK FKPLYKQVLS |
| 301 | DRESLSFYGE GYTSDEEVLE VFRNTLNKNS EIFSSIKKLE KLFKNFDEYS SAGIFVKNGP |
| 361 | AISTISKDIF GEWNVIRDKW NAEYDDIHLK KKAVVTEKYE DDRRKSFKKI GSFSLEQLQE |
| 421 | YADADLSVVE KLKEIIIQKV DEIYKVYGSS EKLFDADFVL EKSLKKNDAV VAIMKDLLDS |
| 481 | VKSFENYIKA FFGEGKETNR DESFYGDFVL AYDILLKVDH IYDAIRNYVT QKPYSKDKFK |
| 541 | LYFQNPQFMG GWDKDKETDY RATILRYGSK YYLAIMDKKY AKCLQKIDKD DVNGNYEKIN |
| 601 | YKLLPGPNKM LPKVFFSKKW MAYYNPSEDI QKIYKNGTFK KGDMFNLNDC HKLIDFFKDS |
| 661 | ISRYPKWSNA YDFNFSETEK YKDIAGFYRE VEEQGYKVSF ESASKKEVDK LVEEGKLYMF |
| 721 | QIYNKDFSDK SHGTPNLHTM YFKLLFDENN HGQIRLSGGA ELFMRRASLK KEELVVHPAN |
| 781 | SPIANKNPDN PKKTTTLSYD VYKDKRFSED QYELHIPIAI NKCPKNIFKI NTEVRVLLKH |
| 841 | DDNPYVIGID RGERNLLYIV VVDGKGNIVE QYSLNEIINN FNGIRIKTDY HSLLDKKEKE |
| 901 | RFEARQNWTS IENIKELKAG YISQVVHKIC ELVEKYDAVI ALEDLNSGFK NSRVKVEKQV |
| 961 | YQKFEKMLID KLNYMVDKKS NPCATGGALK GYQITNKFES FKSMSTQNGF IFYIPAWLTS |
| 1021 | KIDPSTGFVN LLKTKYTSIA DSKKFISSFD RIMYVPEEDL FEFALDYKNF SRTDADYIKK |
| 1081 | WKLYSYGNRI RIFRNPKKNN VFDWEEVCLT SAYKELFNKY GINYQQGDIR ALLCEQSDKA |
| 1141 | FYSSFMALMS LMLQMRNSIT GRTDVDFLIS PVKNSDGIFY DSRNYEAQEN AILPKNADAN |
| 1201 | GAYNIARKVL WAIGQFKKAE DEKLDKVKIA ISNKEWLEYA QTSVKH |

The mature LbCpf1 (without 18 amino acid signal sequence) (SEQ ID NO:4) is as follows:

MSKLEKFTNCYSLSKTLRFKAIPVGKTQENIDNKR
LLVEDEKRAEDYKGVKKLLDRYYLSFINDVLHSIK
LKNLNNYISLFRKKTRTEKENKELENLEINLRKEI
AKAFKGNEGYKSLFKKDIIETILPEFLDDKDEIAL
VNSFNGFTTAFTGFFDNRENMFSEEAKSTSIAFRC
INENLTRYISNMDIFEKVDAIFDKHEVQEIKEKIL
NSDYDVEDFFEGEFFNFVLTQEGIDVYNAIIGGFV
TESGEKIKGLNEYINLYNQKTKQKLPKFKPLYKQV
LSDRESLSFYGEGYTSDEEVLEVFRNTLNKNSEIF
SSIKKLEKLFKNFDEYSSAGIFVKNGPAISTISKD
IFGEWNVIRDKWNAEYDDIHLKKKAVVTEKYEDDR
RKSFKKIGSFSLEQLQEYADADLSVVEKLKEIIIQ
KVDEIYKVYGSSEKLFDADFVLEKSLKKNDAVVAI
MKDLLDSVKSFENYIKAFFGEGKETNRDESFYGDF
VLAYDILLKVDHIYDAIRNYVTQKPYSKDKFKLYF
QNPQFMGGWDKDKETDYRATILRYGSKYYLAIMDK
KYAKCLQKIDKDDVNGNYEKINYKLLPGPNKMLPK
VFFSKKWMAYYNPSEDIQKIYKNGTFKKGDMFNLN
DCHKLIDFFKDSISRYPKWSNAYDFNFSETEKYKD
IAGFYREVEEQGYKVSFESASKKEVDKLVEEGKLY
MFQIYNKDFSDKSHGTPNLHTMYFKLLFDENNHGQ
IRLSGGAELFMRRASLKKEELVVHPANSPIANKNP
DNPKKTTTLSYDVYKDKRFSEDQYELHIPIAINKC
PKNIFKINTEVRVLLKHDDNPYVIGIDRGERNLLY
IVVVDGKGNIVEQYSLNEIINNFNGIRIKTDYHSL
LDKKEKERFEARQNWTSIENIKELKAGYISQVVHK
ICELVEKYDAVIALEDLNSGFKNSRVKVEKQVYQK
FEKMLIDKLNYMVDKKSNPCATGGALKGYQITNKF
ESFKSMSTQNGFIFYIPAWLTSKIDPSTGFVNLLK
TKYTSIADSKKFISSFDRIMYVPEEDLFEFALDYK
NFSRTDADYIKKWKLYSYGNRIRIFRNPKKNNVFD
WEEVCLTSAYKELFNKYGINYQQGDIRALLCEQSD
KAFYSSFMALMSLMLQMRNSITGRTDVDFLISPVK
NSDGIFYDSRNYEAQENAILPKNADANGAYNIARK
VLWAIGQFKKAEDEKLDKVKIAISNKEWLEYAQTS
VKH

The LbCpf1 variants described herein can include the amino acid sequence of SEQ ID NO:3, e.g., at least comprising amino acids 23-1246 of SEQ ID NO:3, with mutations (i.e., replacement of the native amino acid with a different amino acid, e.g., alanine, glycine, or serine), at one or more of the positions in Table 3; amino acids 19-1246 of SEQ ID NO:3 are identical to amino acids 1-1228 of SEQ ID NO:4 (amino acids 1-1246 of SEQ ID NO:3 are referred to herein as LbCPF1 (+18)). In some embodiments, the LbCpf1 variants are at least 80%, e.g., at least 85%, 90%, or 95% identical to the amino acid sequence of SEQ ID NO:4, e.g., have differences at up to 5%, 10%, 15%, or 20% of the residues of SEQ ID NO:4 replaced, e.g., with conservative mutations, in addition to the mutations described herein. In preferred embodiments, the variant retains desired activity of the parent, e.g., the nuclease activity (except where the parent is a nickase or a dead Cpf1), and/or the ability to interact with a guide RNA and target DNA). The LbCpf1 variant can be SEQ ID NO:4, omitting the first 18 amino acids boxed above as described in Zetsche et al. Cell 163, 759-771 (2015).

In some embodiments, the Cpf1 variants also include one of the following mutations listed in Table 3, which reduce or destroy the nuclease activity of the Cpf1 (i.e., render them catalytically inactive):

TABLE 3

| | |
|---|---|
| LbCpf1 (+18) | LbCpf1 |

Residues involved in DNA and RNA catalysis

| | | |
|---|---|---|
| DNA targeting | D850 | D832 |
| | E853 | E835 |
| | N855 | N837 |
| | Y858 | Y840 |
| | E943 | E925 |
| | R1156 | R1138 |
| | S1158 | S1140 |
| | D1166 | D1148 |
| | D1198 | D1180 |
| RNA processing | H777 | H759 |
| | K786 | K768 |
| | K803 | K785 |
| | F807 | F789 |

Mutations that turn Cpf1 into a nickase

| | |
|---|---|
| R1156A | R1138A |

See, e.g., Yamano et al., Cell. 2016 May 5; 165(4):949-62; Fonfara et al., Nature. 2016 Apr. 28; 532(7600):517-21; Dong et al., Nature. 2016 Apr. 28; 532(7600):522-6; and Zetsche et al., Cell. 2015 Oct. 22; 163(3):759-71. Note that “LbCpf1 (+18)” refers to the full sequence of amino acids 1-1246 of SEQ ID NO:3, while the LbCpf1 refers to the sequence of LbCpf1 in Zetsche et al., also shown herein as amino acids 1-1228 of SEQ ID NO:4 and amino acids 19-1246 of SEQ ID NO:3. Thus, in some embodiments, for LbCpf1 catalytic activity-destroying mutations are made at D832 and E925, e.g., D832A and E925A.

### TAL Effector Repeat Arrays

Transcription activator like effectors (TALEs) of plant pathogenic bacteria in the genus Xanthomonas play important roles in disease, or trigger defense, by binding host DNA and activating effector-specific host genes. Specificity depends on an effector-variable number of imperfect, typically ˜33-35 amino acid repeats. Polymorphisms are present primarily at repeat positions 12 and 13, which are referred to herein as the repeat variable-diresidue (RVD). The RVDs of TAL effectors correspond to the nucleotides in their target sites in a direct, linear fashion, one RVD to one nucleotide, with some degeneracy and no apparent context dependence. In some embodiments, the polymorphic region that grants nucleotide specificity can be expressed as a triresidue or triplet.

Each DNA binding repeat can include a RVD that determines recognition of a base pair in the target DNA sequence, wherein each DNA binding repeat is responsible for recognizing one base pair in the target DNA sequence. In some embodiments, the RVD can comprise one or more of: HA for recognizing C; ND for recognizing C; HI for recognizing C; HN for recognizing G; NA for recognizing G; SN for recognizing G or A; YG for recognizing T; and NK for recognizing G, and one or more of: HD for recognizing C; NG for recognizing T; NI for recognizing A; NN for recognizing G or A; NS for recognizing A or C or G or T; N* for recognizing C or T, wherein * represents a gap in the second position of the RVD; HG for recognizing T; H* for recognizing T, wherein * represents a gap in the second position of the RVD; and IG for recognizing T.

TALE proteins can be useful in research and biotechnology as targeted chimeric nucleases that can facilitate homologous recombination in genome engineering (e.g., to add or enhance traits useful for biofuels or biorenewables in plants). These proteins also can be useful as, for example, transcription factors, and especially for therapeutic applications requiring a very high level of specificity such as therapeutics against pathogens (e.g., viruses) as non-limiting examples.

Methods for generating engineered TALE arrays are known in the art, see, e.g., the fast ligation-based automatable solid-phase high-throughput (FLASH) system described in U.S. Ser. No. 61/610,212, and Reyon et al., Nature Biotechnology 30, 460-465 (2012); as well as the methods described in Bogdanove & Voytas, Science 333, 1843-1846 (2011); Bogdanove et al., Curr Opin Plant Biol 13, 394-401 (2010); Scholze & Boch, J. Curr Opin Microbiol (2011); Boch et al., Science 326, 1509-1512 (2009); Moscou & Bogdanove, Science 326, 1501 (2009); Miller et al., Nat Biotechnol 29, 143-148 (2011); Morbitzer et al., T. Proc Natl Acad Sci USA 107, 21617-21622 (2010); Morbitzer et al., Nucleic Acids Res 39, 5790-5799 (2011); Zhang et al., Nat Biotechnol 29, 149-153 (2011); Geissler et al., PLoS ONE 6, e19509 (2011); Weber et al., PLoS ONE 6, e19722 (2011); Christian et al., Genetics 186, 757-761 (2010); Li et al., Nucleic Acids Res 39, 359-372 (2011); Mahfouz et al., Proc Natl Acad Sci USA 108, 2623-2628 (2011); Mussolino et al., Nucleic Acids Res (2011); Li et al., Nucleic Acids Res 39, 6315-6325 (2011); Cermak et al., Nucleic Acids Res 39, e82 (2011); Wood et al., Science 333, 307 (2011); Hockemeye et al. Nat Biotechnol 29, 731-734 (2011); Tesson et al., Nat Biotechnol 29, 695-696 (2011); Sander et al., Nat Biotechnol 29, 697-698 (2011); Huang et al., Nat Biotechnol 29, 699-700 (2011); and Zhang et al., Nat Biotechnol 29, 149-153 (2011); all of which are incorporated herein by reference in their entirety.

### Zinc Fingers

Zinc finger (ZF) proteins are DNA-binding proteins that contain one or more zinc fingers, independently folded zinc-containing mini-domains, the structure of which is well known in the art and defined in, for example, Miller et al., 1985, EMBO J., 4:1609; Berg, 1988, Proc. Natl. Acad. Sci. USA, 85:99; Lee et al., 1989, Science. 245:635; and Klug, 1993, Gene, 135:83. Crystal structures of the zinc finger protein Zif268 and its variants bound to DNA show a semi-conserved pattern of interactions, in which typically three amino acids from the alpha-helix of the zinc finger contact three adjacent base pairs or a “subsite” in the DNA (Pavletich et al., 1991, Science, 252:809; Elrod-Erickson et al., 1998, Structure, 6:451). Thus, the crystal structure of Zif268 suggested that zinc finger DNA-binding domains might function in a modular manner with a one-to-one interaction between a zinc finger and a three-base-pair “subsite” in the DNA sequence. In naturally occurring zinc finger transcription factors, multiple zinc fingers are typically linked together in a tandem array to achieve sequence-specific recognition of a contiguous DNA sequence (Klug, 1993, Gene 135:83).

Multiple studies have shown that it is possible to artificially engineer the DNA binding characteristics of individual zinc fingers by randomizing the amino acids at the alpha-helical positions involved in DNA binding and using selection methodologies such as phage display to identify desired variants capable of binding to DNA target sites of interest (Rebar et al., 1994, Science, 263:671; Choo et al., 1994 Proc. Natl. Acad. Sci. USA, 91:11163; Jamieson et al., 1994, Biochemistry 33:5689; Wu et al., 1995 Proc. Natl. Acad. Sci. USA, 92: 344). Such recombinant zinc finger proteins can be fused to functional domains, such as transcriptional activators, transcriptional repressors, methylation domains, and nucleases to regulate gene expression, alter DNA methylation, and introduce targeted alterations into genomes of model organisms, plants, and human cells (Carroll, 2008, Gene Ther., 15:1463-68; Cathomen, 2008, Mol. Ther., 16:1200-07; Wu et al., 2007, Cell. Mol. Life Sci., 64:2933-44).

One existing method for engineering zinc finger arrays, known as “modular assembly,” advocates the simple joining together of pre-selected zinc finger modules into arrays (Segal et al., 2003, Biochemistry, 42:2137-48; Beerli et al., 2002, Nat. Biotechnol., 20:135-141; Mandell et al., 2006, Nucleic Acids Res., 34:W516-523; Carroll et al., 2006, Nat. Protoc. 1:1329-41; Liu et al., 2002, J. Biol. Chem., 277:3850-56; Bae et al., 2003, Nat. Biotechnol., 21:275-280; Wright et al., 2006, Nat. Protoc., 1:1637-52). Although straightforward enough to be practiced by any researcher, reports have demonstrated a high failure rate for this method, particularly in the context of zinc finger nucleases (Ramirez et al., 2008, Nat. Methods, 5:374-375; Kim et al., 2009, Genome Res. 19:1279-88), a limitation that typically necessitates the construction and cell-based testing of very large numbers of zinc finger proteins for any given target gene (Kim et al., 2009, Genome Res. 19:1279-88).

Combinatorial selection-based methods that identify zinc finger arrays from randomized libraries have been shown to have higher success rates than modular assembly (Maeder et al., 2008, Mol. Cell, 31:294-301; Joung et al., 2010, Nat. Methods, 7:91-92; Isalan et al., 2001, Nat. Biotechnol., 19:656-660). In preferred embodiments, the zinc finger arrays are described in, or are generated as described in, WO 2011/017293 and WO 2004/099366. Additional suitable zinc finger DBDs are described in U.S. Pat. Nos. 6,511,808, 6,013,453, 6,007,988, and 6,503,717 and U.S. patent application 2002/0160940.

### Gene Expression Modulating Domains

The aTFs described herein can also include a gene expression modulation domain. In some embodiments, the gene expression modulation domain is a gene expression activation domain (e.g., a transcription activation domain of a transcription factor). Non-limiting examples of gene expression activation domain include activation domains of NF-κB (e.g., p65), VP40, VPR, or p300. The gene expression modulation domain can also be a protein that can introduce or remove covalent modifications to histones or DNA. Non-limiting examples of such proteins could include LSD1 or TET1. The gene expression modulation domain could also be a protein that recruits (either directly or indirectly) other proteins in the cell that in turn can modulate gene expression.

In some embodiments, the gene expression modulating domain is a heterologous functional domain (HFD) that modifies gene expression, histones, or DNA, e.g., transcriptional activation domain, transcriptional repressors (e.g., silencers such as Heterochromatin Protein 1 (HP1), e.g., HP1α or HP1β, or a transcriptional repression domain, e.g., Krueppel-associated box (KRAB) domain, ERF repressor domain (ERD), or mSin3A interaction domain (SID)), enzymes that modify the methylation state of DNA (e.g., DNA methyltransferase (DNMT) or Ten-Eleven Translocation (TET) proteins, e.g., TET1, also known as Tet Methylcytosine Dioxygenase 1), or enzymes that modify histone subunit (e.g., histone acetyltransferases (HAT), histone deacetylases (HDAC), or histone demethylases). In some embodiments, the heterologous functional domain is a transcriptional activation domain, e.g., a transcriptional activation domain from VP64 or NF-κB p65; an enzyme that catalyzes DNA demethylation, e.g., a TET; or histone modification (e.g., LSD1, histone methyltransferase, HDACs, or HATs) or a transcription silencing domain, e.g., from Heterochromatin Protein 1 (HP1), e.g., HP1α or HP1β; or a biological tether, e.g., CRISPR/Cas Subtype Ypest protein 4 (Csy4), MS2,or lambda N protein.

In some embodiments, the heterologous functional domain is linked to the N terminus or C terminus of the catalytically inactive Cas9 protein, with an optional intervening linker, wherein the linker does not interfere with activity of the fusion protein.

The transcriptional activation domains can be fused on the N or C terminus of the Cas9. In addition, although the present description exemplifies transcriptional activation domains, other heterologous functional domains (e.g., transcriptional repressors (e.g., KRAB, ERD, SID, and others, e.g., amino acids 473-530 of the ets2 repressor factor (ERF) repressor domain (ERD), amino acids 1-97 of the KRAB domain of KOX1, or amino acids 1-36 of the Mad mSIN3 interaction domain (SID); see Beerli et al., PNAS USA 95:14628-14633 (1998)) or silencers such as Heterochromatin Protein 1 (HP1, also known as swi6), e.g., HP1α or HP1β; proteins or peptides that could recruit long non-coding RNAs (lncRNAs) fused to a fixed RNA binding sequence such as those bound by the MS2 coat protein, endoribonuclease Csy4, or the lambda N protein; enzymes that modify the methylation state of DNA (e.g., DNA methyltransferase (DNMT) or TET proteins); or enzymes that modify histone subunits (e.g., histone acetyltransferases (HAT), histone deacetylases (HDAC), histone methyltransferases (e.g., for methylation of lysine or arginine residues) or histone demethylases (e.g., for demethylation of lysine or arginine residues)) as are known in the art can also be used. A number of sequences for such domains are known in the art, e.g., a domain that catalyzes hydroxylation of methylated cytosines in DNA. Exemplary proteins include the Ten-Eleven-Translocation (TET)1-3 family, enzymes that converts 5-methylcytosine (5-mC) to 5-hydroxymethylcytosine (5-hmC) in DNA.

Sequences for human TET1-3 are known in the art and are shown in the following table:

GenBank Accession Nos.

| Gene | Amino Acid | Nucleic Acid |
|---|---|---|
| TET1 | NP_085128.2 | NM_030625.2 |
| TET2* | NP_001120680.1 (var 1) | NM_001127208.2 |
| | NP_060098.3 (var 2) | NM_017628.4 |
| TET3 | NP_659430.1 | NM_144993.1 |

*Variant (1) represents the longer transcript and encodes the longer isoform (a). Variant (2) differs in the 5′ UTR and in the 3′ UTR and coding sequence compared to variant 1. The resulting isoform (b) is shorter and has a distinct C-terminus compared to isoform a.

In some embodiments, all or part of the full-length sequence of the catalytic domain can be included, e.g., a catalytic module comprising the cysteine-rich extension and the 2OGFeDO domain encoded by 7 highly conserved exons, e.g., the Tet1 catalytic domain comprising amino acids 1580-2052, Tet2 comprising amino acids 1290-1905 and Tet3 comprising amino acids 966-1678. See, e.g., FIG. 1 of Iyer et al., Cell Cycle. 2009 Jun. 1; 8(11):1698-710. Epub 2009 Jun. 27, for an alignment illustrating the key catalytic residues in all three Tet proteins, and the supplementary materials thereof (available at ftp site ftp.ncbi.nih.gov/pub/aravind/DONS/supplementary_material_DONS.html) for full length sequences (see, e.g., seq 2c); in some embodiments, the sequence includes amino acids 1418-2136 of Tet1 or the corresponding region in Tet2/3.

Other catalytic modules can be from the proteins identified in Iyer et al., 2009.

In some embodiments, the heterologous functional domain is a biological tether, and comprises all or part of (e.g., DNA binding domain from) the MS2 coat protein, endoribonuclease Csy4, or the lambda N protein. These proteins can be used to recruit RNA molecules containing a specific stem-loop structure to a locale specified by the dCas9 gRNA targeting sequences. For example, a dCas9 fused to MS2 coat protein, endoribonuclease Csy4, or lambda N can be used to recruit a long non-coding RNA (lncRNA) such as XIST or HOTAIR; see, e.g., Keryer-Bibens et al., Biol. Cell 100:125-138 (2008), that is linked to the Csy4, MS2 or lambda N binding sequence. Alternatively, the Csy4, MS2 or lambda N protein binding sequence can be linked to another protein, e.g., as described in Keryer-Bibens et al., supra, and the protein can be targeted to the dCas9 binding site using the methods and compositions described herein. In some embodiments, the Csy4 is catalytically inactive. In some embodiments, the Csy4 is catalytically inactive. In some embodiments, the Cas9 variant, preferably a dCas9 variant, is fused to FokI as described in U.S. Pat. No. 8,993,233; US 20140186958; U.S. Pat. No. 9,023,649; WO/2014/099744; WO 2014/089290; WO2014/144592; WO144288; WO2014/204578; WO2014/152432; WO2115/099850; U.S. Pat. No. 8,697,359; US2010/0076057; US2011/0189776; US2011/0223638; US2013/0130248; WO/2008/108989; WO/2010/054108; WO/2012/164565; WO/2013/098244; WO/2013/176772; US20150050699; US 20150071899 and WO 2014/204578.

In some embodiments, the fusion proteins include a linker between the dCas9 and the heterologous functional domains. Linkers that can be used in these fusion proteins (or between fusion proteins in a concatenated structure) can include any sequence that does not interfere with the function of the fusion proteins. In preferred embodiments, the linkers are short, e.g., 2-20 amino acids, and are typically flexible (i.e., comprising amino acids with a high degree of freedom such as glycine, alanine, and serine). In some embodiments, the linker comprises one or more units consisting of GGGS (SEQ ID NO:5) or GGGGS (SEQ ID NO:6), e.g., two, three, four, or more repeats of the GGGS (SEQ ID NO:5) or GGGGS (SEQ ID NO:6) unit. Other linker sequences can also be used.

Guide RNA (gRNA)

In some embodiments, e.g., when the aTF is a CRISPR based aTF, the aTF system comprises one or more nucleic acids encoding gRNA(s), e.g., enhancer targeting and/or promoter targeting gRNA(s). Suitable gRNA are those that target a nucleic acid sequence binding domain, e.g. CRISPR-Cas or CRISPR-Cpf1, to a selected sequence e.g., a promotor or enhancer.

In some embodiments, the gRNA is specific to a particular promoter or enhancer sequence. In some embodiments, the gRNA is specific to a particular allele of the promoter or enhancer sequence.

In some embodiments, the guide RNAs can interact with the Cas and/or Cpf1 protein and direct it to the target sequence (e.g., the promoter or enhancer)

The gRNA(s) can be encoded on one or more expression vectors. Thus, in some embodiments, the aTFs described herein comprise one or more nucleic acid vector(s) encoding gRNA(s). The nucleic acid vector(s) encoding gRNA(s) can also encode other elements of the aTFs described herein, e.g., fusion proteins, e.g., Cas9 or Cpf1 fusion proteins.

### Methods of Use

The described aTF systems are useful and versatile tools for modifying gene expression, e.g., the expression of endogenous genes. Current methods for achieving this require the generation of novel engineered DNA-binding proteins (such as engineered zinc finger or transcription activator-like effector DNA binding domains) for each site to be targeted. Because these methods demand expression of a large protein specifically engineered to bind each target site, they are limited in their capacity for multiplexing. aTFs, however, require expression of only a single Cas9-gene expression domain fusion protein, which can be targeted to multiple sites in the genome by expression of multiple short gRNAs. This system could therefore easily be used to simultaneously induce expression of a large number of genes or to recruit multiple Cas9-gene expression domain fusion proteins to a single gene, promoter, or enhancer. This capability will have broad utility, e.g., for basic biological research, where it can be used to study gene function and to manipulate the expression of multiple genes in a single pathway, and in synthetic biology, where it will enable researchers to create circuits in cell that are responsive to multiple input signals. The relative ease with which this technology can be implemented and adapted to multiplexing will make it a broadly useful technology with many wide-ranging applications.

The methods described herein include contacting cells with a nucleic acid encoding the fusion proteins described herein, and nucleic acids encoding one or more guide RNAs directed to a selected gene, to thereby modulate expression of that gene.

Guide RNAs (gRNAs)

Guide RNAs generally speaking come in two different systems: System 1, which uses separate crRNA and tracrRNAs that function together to guide cleavage by Cas9, and System 2, which uses a chimeric crRNA-tracrRNA hybrid that combines the two separate guide RNAs in a single system (referred to as a single guide RNA or sgRNA, see also Jinek et al., Science 2012; 337:816-821). The tracrRNA can be variably truncated and a range of lengths has been shown to function in both the separate system (system 1) and the chimeric gRNA system (system 2). For example, in some embodiments, tracrRNA may be truncated from its 3′ end by at least 1, 2, 3, 4, 5, 6, 7, 8, 9, 10, 15, 20, 25, 30, 35 or 40 nts. In some embodiments, the tracrRNA molecule may be truncated from its 5′ end by at least 1, 2, 3, 4, 5, 6, 7, 8, 9, 10, 15, 20, 25, 30, 35 or 40 nts. Alternatively, the tracrRNA molecule may be truncated from both the 5′ and 3′ end, e.g., by at least 1, 2, 3, 4, 5, 6, 7, 8, 9, 10, 15 or 20 nts on the 5′ end and at least 1, 2, 3, 4, 5, 6, 7, 8, 9, 10, 15, 20, 25, 30, 35 or 40 nts on the 3′ end. See, e.g., Jinek et al., Science 2012; 337:816-821; Mali et al., Science. 2013 Feb. 15; 339(6121):823-6; Cong et al., Science. 2013 Feb. 15; 339(6121):819-23; and Hwang and Fu et al., Nat Biotechnol. 2013 March; 31(3):227-9; Jinek et al., Elife 2, e00471 (2013)). For System 2, generally the longer length chimeric gRNAs have shown greater on-target activity but the relative specificities of the various length gRNAs currently remain undefined and therefore it may be desirable in certain instances to use shorter gRNAs. In some embodiments, the gRNAs are complementary to a region that is within about 100-800 bp upstream of the transcription start site, e.g., is within about 500 bp upstream of the transcription start site, includes the transcription start site, or within about 100-800 bp, e.g., within about 500 bp, downstream of the transcription start site. In some embodiments, vectors (e.g., plasmids) encoding more than one gRNA are used, e.g., plasmids encoding, 2, 3, 4, 5, or more gRNAs directed to different sites in the same region of the target gene.

Cas9 nuclease can be guided to specific 17-20 nt genomic targets bearing an additional proximal protospacer adjacent motif (PAM), e.g., of sequence NGG, using a guide RNA, e.g., a single gRNA or a tracrRNA/crRNA, bearing 17-20 nts at its 5′ end that are complementary to the complementary strand of the genomic DNA target site. Thus, the present methods can include the use of a single guide RNA comprising a crRNA fused to a normally trans-encoded tracrRNA, e.g., a single Cas9 guide RNA as described in Mali et al., Science 2013 Feb. 15; 339(6121):823-6, with a sequence at the 5′ end that is complementary to the target sequence, e.g., of 25-17, optionally 20 or fewer nucleotides (nts), e.g., 20, 19, 18, or 17 nts, preferably 17 or 18 nts, of the complementary strand to a target sequence immediately 5′ of a protospacer adjacent motif (PAM), e.g., NGG, NAG, or NNGG. In some embodiments, the single Cas9 guide RNA consists of the sequence:

(SEQ ID NO: 209)
(X₁₇₋₂₀)GUUUUAGAGCUAGAAAUAGCAAG
UUAAAAUAAGGCUAGUCCG(X_{N});
(SEQ ID NO: 210)
(X₁₇₋₂₀)GUUUUAGAGCUAUGCUGAAAAGC
AUAGCAAGUUAAAAUAAGGCUAGUCCGUU
AUC(X_{N});
(SEQ ID NO: 211)
(X₁₇₋₂₀)GUUUUAGAGCUAUGCUGUUUUGGA
AACAAAACAGCAUAGCAAGUUAAAAUAAGG
CUAGUCCGUUAUC(X_{N});
(SEQ ID NO: 212)
(X₁₇₋₂₀)GUUUUAGAGCUAGAAAUAGCAAGU
UAAAAUAAGGCUAGUCCGUUAUCAACUUGA
AAAAGUGGCACCGAGUCGGUGC(X_{N}),
(SEQ ID NO: 213)
(X₁₇₋₂₀)GUUUAAGAGCUAGAAAUAGCAAGU
UUAAAUAAGGCUAGUCCGUUAUCAACUUGA
AAAAGUGGCACCGAGUCGGUGC;
(SEQ ID NO: 214)
(X₁₇₋₂₀)GUUUUAGAGCUAUGCUGGAAACAG
CAUAGCAAGUUUAAAUAAGGCUAGUCCGUU
AUCAACUUGAAAAAGUGGCACCGAGUCGGUG
C;
or
(SEQ ID NO: 215)
(X₁₇₋₂₀)GUUUAAGAGCUAUGCUGGAAACAG
CAUAGCAAGUUUAAAUAAGGCUAGUCCGUU
AUCAACUUGAAAAAGUGGCACCGAGUCGGUG
C;

wherein X₁₇₋₂₀ is the nucleotide sequence complementary to 17-20 consecutive nucleotides of the target sequence. DNAs encoding the single guide RNAs have been described previously in the literature (Jinek et al., Science. 337(6096):816-21 (2012) and Jinek et al., Elife. 2:e00471 (2013)).

The guide RNAs can include X_{N} which can be any sequence, wherein N (in the RNA) can be 0-200, e.g., 0-100, 0-50, or 0-20, that does not interfere with the binding of the ribonucleic acid to Cas9.

In some embodiments, the guide RNA includes one or more Adenine (A) or Uracil (U) nucleotides on the 3′ end. In some embodiments the RNA includes one or more U, e.g., 1 to 8 or more Us (e.g., U, UU, UUU, UUUU, UUUUU, UUUUUU, UUUUUUU,) at the 3′ end of the molecule, as a result of the optional presence of one or more Ts used as a termination signal to terminate RNA PolIII transcription.

Although some of the examples described herein utilize a single gRNA, the methods can also be used with dual gRNAs (e.g., the crRNA and tracrRNA found in naturally occurring systems). In this case, a single tracrRNA would be used in conjunction with multiple different crRNAs expressed using the present system, e.g., the following:
(X₁₇₋₂₀)GUUUUAGAGCUA (SEQ ID NO:216);
(X₁₇₋₂₀) GUUUUAGAGCUAUGCUGUUUUG (SEQ ID NO:217); or
(X₁₇₋₂₀)GUUUUAGAGCUAUGCU (SEQ ID NO:218); and a tracrRNA sequence. In this case, the crRNA is used as the guide RNA in the methods and molecules described herein, and the tracrRNA can be expressed from the same or a different DNA molecule. In some embodiments, the methods include contacting the cell with a tracrRNA comprising or consisting of the sequence GGAACCAUUCAAAACAGCAUAGCAAGUUAAAAUAAGGCUAGUCCGUUA UCAACUUGAAAAAGUGGCACCGAGUCGGUGC (SEQ ID NO:219) or an active portion thereof (an active portion is one that retains the ability to form complexes with Cas9 or dCas9). In some embodiments, the tracrRNA molecule may be truncated from its 3′ end by at least 1, 2, 3, 4, 5, 6, 7, 8, 9, 10, 15, 20, 25, 30, 35 or 40 nts. In another embodiment, the tracrRNA molecule may be truncated from its 5′ end by at least 1, 2, 3, 4, 5, 6, 7, 8, 9, 10, 15, 20, 25, 30, 35 or 40 nts. Alternatively, the tracrRNA molecule may be truncated from both the 5′ and 3′ end, e.g., by at least 1, 2, 3, 4, 5, 6, 7, 8, 9, 10, 15 or 20 nts on the 5′ end and at least 1, 2, 3, 4, 5, 6, 7, 8, 9, 10, 15, 20, 25, 30, 35 or 40 nts on the 3′ end. Exemplary tracrRNA sequences in addition to SEQ ID NO:219 include the following: UAGCAAGUUAAAAUAAGGCUAGUCCGUUAUCAACUUGAAAAAGUGGCA CCGAGUCGGUGC (SEQ ID NO:220) or an active portion thereof; or AGCAUAGCAAGUUAAAAUAAGGCUAGUCCGUUAUCAACUUGAAAAAGU GGCACCGAGUCGGUGC (SEQ ID NO:221) or an active portion thereof.

In some embodiments when (X₁₇₋₂₀)GUUUUAGAGCUAUGCUGUUUUG (SEQ ID NO:222) is used as a crRNA, the following tracrRNA is used: GGAACCAUUCAAAACAGCAUAGCAAGUUAAAAUAAGGCUAGUCCGUUA UCAACUUGAAAAAGUGGCACCGAGUCGGUGC (SEQ ID NO:223) or an active portion thereof.

In some embodiments when (X₁₇₋₂₀)GUUUUAGAGCUA (SEQ ID NO:224) is used as a crRNA, the following tracrRNA is used: UAGCAAGUUAAAAUAAGGCUAGUCCGUUAUCAACUUGAAAAAGUGGCA CCGAGUCGGUGC (SEQ ID NO:225) or an active portion thereof.

In some embodiments when (X₁₇₋₂₀) GUUUUAGAGCUAUGCU (SEQ ID NO:226) is used as a crRNA, the following tracrRNA is used: AGCAUAGCAAGUUAAAAUAAGGCUAGUCCGUUAUCAACUUGAAAAAGU GGCACCGAGUCGGUGC (SEQ ID NO:227) or an active portion thereof.

In some embodiments, the gRNA is targeted to a site that is at least three or more mismatches different from any sequence in the rest of the genome in order to minimize off-target effects.

Modified RNA oligonucleotides such as locked nucleic acids (LNAs) have been demonstrated to increase the specificity of RNA-DNA hybridization by locking the modified oligonucleotides in a more favorable (stable) conformation. For example, 2′-O-methyl RNA is a modified base where there is an additional covalent linkage between the 2′ oxygen and 4′ carbon which when incorporated into oligonucleotides can improve overall thermal stability and selectivity (Formula I).

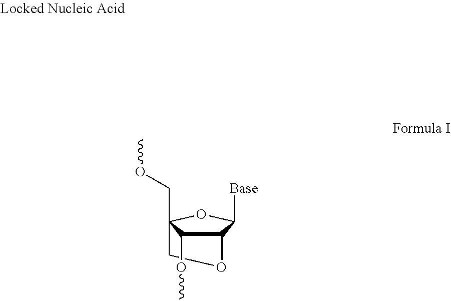

Thus in some embodiments, the tru-gRNAs disclosed herein may comprise one or more modified RNA oligonucleotides. For example, the truncated guide RNAs molecules described herein can have one, some or all of the region of the guideRNA complementary to the target sequence are modified, e.g., locked (2′-O-4′-C methylene bridge), 5′-methylcytidine, 2′-O-methyl-pseudouridine, or in which the ribose phosphate backbone has been replaced by a polyamide chain (peptide nucleic acid), e.g., a synthetic ribonucleic acid.

In other embodiments, one, some or all of the nucleotides of the tru-gRNA sequence may be modified, e.g., locked (2′-O-4′-C methylene bridge), 5′-methylcytidine, 2′-O-methyl-pseudouridine, or in which the ribose phosphate backbone has been replaced by a polyamide chain (peptide nucleic acid), e.g., a synthetic ribonucleic acid.

In some embodiments, the single guide RNAs and/or crRNAs and/or tracrRNAs can include one or more Adenine (A) or Uracil (U) nucleotides on the 3′ end.

Existing Cas9-based RGNs use gRNA-DNA heteroduplex formation to guide targeting to genomic sites of interest. However, RNA-DNA heteroduplexes can form a more promiscuous range of structures than their DNA-DNA counterparts. In effect, DNA-DNA duplexes are more sensitive to mismatches, suggesting that a DNA-guided nuclease may not bind as readily to off-target sequences, making them comparatively more specific than RNA-guided nucleases. Thus, the guide RNAs usable in the methods described herein can be hybrids, i.e., wherein one or more deoxyribonucleotides, e.g., a short DNA oligonucleotide, replaces all or part of the gRNA, e.g., all or part of the complementarity region of a gRNA. This DNA-based molecule could replace either all or part of the gRNA in a single gRNA system or alternatively might replace all of part of the crRNA and/or tracrRNA in a dual crRNA/tracrRNA system. Such a system that incorporates DNA into the complementarity region should more reliably target the intended genomic DNA sequences due to the general intolerance of DNA-DNA duplexes to mismatching compared to RNA-DNA duplexes. Methods for making such duplexes are known in the art, See, e.g., Barker et al., BMC Genomics. 2005 Apr. 22; 6:57; and Sugimoto et al., Biochemistry. 2000 Sep. 19; 39(37):11270-81.

In addition, in a system that uses separate crRNA and tracrRNA, one or both can be synthetic and include one or more modified (e.g., locked) nucleotides or deoxyribonucleotides.

In a cellular context, complexes of Cas9 with these synthetic gRNAs could be used to improve the genome-wide specificity of the CRISPR/Cas9 nuclease system.

The methods described can include expressing in a cell, or contacting the cell with, a Cas9 gRNA plus a fusion protein as described herein.

### Enhancer and Promoter Regions

Enhancer regions are regulatory sequences generally located far from the promoters that they regulate. See, e.g., Bulger and Groudine, “Enhancers: The Abundance and Function of Regulatory Sequences beyond Promoters,” Developmental Biology 339(2):250-7 (2010); and Spitz and Furlong, “Transcription Factors: From Enhancer Binding to Developmental Control,” Nature Reviews Genetics 13:613-26 (2012).

Enhancer regions can be downstream or upstream of promoter regions and can be capable of activating transcription regardless of how far they are located from a promoter.

The enhancer regions described herein can be identified, e.g., by functional assays or predictive assays. In some embodiments, the enhancer region is a putative enhancer region, e.g., identified by characteristic(s) associated with enhancer regions, e.g., bioinformatically.

In some embodiments, the enhancer region is identified by monomethylation at histone H3 lysine 4 (H3K4). In some embodiments, the enhancer region is identified by binding with transcriptional coactivator p300.

In some embodiments, the enhancer can encompass putative enhancers (e.g., sequences that contain DNase hypersensitivity sites, those identified as putative enhancer sequences by chromosome conformation capture assay, circularized chromosome conformation capture assay, or Hi-C assay) or those sequences that are upstream or downstream of known enhancer sequence (e.g., within 10 bases, within 100 bases, within 500 bases, or within 1000 bases upstream or downstream from a known enhancer).

In some embodiments, the enhancer region is about 1,000 kb or more away from the transcription start site of the target gene (TSS).

Enhancer regions, e.g., human enhancer regions, are known in the art and described, e.g., in Wang et al., “HACER: an Atlas of Human Active Enhancers to Interpret Regulatory Variants,” Nucleic Acids Research 47(D1):D106-12 (2019) and the HACER database (bioinfo.vanderbilt.edu/AE/HACER/).

Promoter regions, sometimes called core promoters, are the region of a gene to which RNA polymerase II and the general transcription factors (GTFs) bind to initiate transcription. See Spitz and Furlong, “Transcription Factors: From Enhancer Binding to Developmental Control,” Nature Reviews Genetics 13:613-26 (2012). Core promoters span ˜40 base pairs upstream and downstream of the transcription start site. Id.

The promoter regions described herein can be identified, e.g., by functional assays or predictive assays. In some embodiments, the enhancer region is a putative enhancer region, e.g., identified by characteristic(s) associated with enhancer regions, e.g., bioinformatically.

In some embodiments, the promoter region is identified by chromatin immunoprecipitation. In some embodiments, the promoter region is identified bioinformatically.

In some embodiments, the promoter region is between about 1,000 bp upstream to about 500 bp downstream of the transcription start site (TSS) of the target gene. In some embodiments, the promoter is about 500 bp upstream to about 500 bp downstream of the transcription start site (TSS) of the target gene.

Promoter regions, e.g., eukaryotic promoter regions, are known in the art and described, e.g., in Dreos et al., “The Eukaryotic Promoter Database: Expansion of EPDnew and New Promoter Analysis Tools,” Nucleic Acids Research 43(D1):D92-6 (2015) and the Eukaryotic Promoter Database (epd.epfl.ch/index.php).

### Fusion Proteins

The nucleic acid sequence binding domains and gene expression modulating domains disclosed herein can be expressed as part of a fusion protein(s). Also provided herein are isolated nucleic acids encoding the fusion proteins, vectors comprising the isolated nucleic acids, optionally operably linked to one or more regulatory domains for expressing the fusion proteins, and host cells, e.g., mammalian host cells, comprising the nucleic acids, and optionally expressing the fusion proteins.

The fusion proteins described herein can be used for altering the genome of a cell; the methods generally include expressing the variant proteins in the cells, along with a guide RNA having a region complementary to a selected portion of the genome of the cell. Methods for selectively altering the genome of a cell are known in the art, see, e.g., U.S. Pat. No. 8,993,233; US 20140186958; U.S. Pat. No. 9,023,649; WO/2014/099744; WO 2014/089290; WO2014/144592; WO144288; WO2014/204578; WO2014/152432; WO2115/099850; U.S. Pat. No. 8,697,359; US20160024529; US20160024524; US20160024523; US20160024510; US20160017366; US20160017301; US20150376652; US20150356239; US20150315576; US20150291965; US20150252358; US20150247150; US20150232883; US20150232882; US20150203872; US20150191744; US20150184139; US20150176064; US20150167000; US20150166969; US20150159175; US20150159174; US20150093473; US20150079681; US20150067922; US20150056629; US20150044772; US20150024500; US20150024499; US20150020223; US20140356867; US20140295557; US20140273235; US20140273226; US20140273037; US20140189896; US20140113376; US20140093941; US20130330778; US20130288251; US20120088676; US20110300538; US20110236530; US20110217739; US20110002889; US20100076057; US20110189776; US20110223638; US20130130248; US20150050699; US20150071899; US20150045546; US20150031134; US20150024500; US20140377868; US20140357530; US20140349400; US20140335620; US20140335063; US20140315985; US20140310830; US20140310828; US20140309487; US20140304853; US20140298547; US20140295556; US20140294773; US20140287938; US20140273234; US20140273232; US20140273231; US20140273230; US20140271987; US20140256046; US20140248702; US20140242702; US20140242700; US20140242699; US20140242664; US20140234972; US20140227787; US20140212869; US20140201857; US20140199767; US20140189896; US20140186958; US20140186919; US20140186843; US20140179770; US20140179006; US20140170753; WO/2008/108989; WO/2010/054108; WO/2012/164565; WO/2013/098244; WO/2013/176772; Makarova et al., “Evolution and classification of the CRISPR-Cas systems” 9(6) Nature Reviews Microbiology 467-477 (1-23) (June 2011); Wiedenheft et al., “RNA-guided genetic silencing systems in bacteria and archaea” 482 Nature 331-338 (Feb. 16, 2012); Gasiunas et al., “Cas9-crRNA ribonucleoprotein complex mediates specific DNA cleavage for adaptive immunity in bacteria” 109(39) Proceedings of the National Academy of Sciences USA E2579-E2586 (Sep. 4, 2012); Jinek et al., “A Programmable Dual-RNA-Guided DNA Endonuclease in Adaptive Bacterial Immunity” 337 Science 816-821 (Aug. 17, 2012); Carroll, “A CRISPR Approach to Gene Targeting” 20(9) Molecular Therapy 1658-1660 (September 2012); U.S. Appl. No. 61/652,086, filed May 25, 2012; Al-Attar et al., Clustered Regularly Interspaced Short Palindromic Repeats (CRISPRs): The Hallmark of an Ingenious Antiviral Defense Mechanism in Prokaryotes, Biol Chem. (2011) vol. 392, Issue 4, pp. 277-289; Hale et al., Essential Features and Rational Design of CRISPR RNAs That Function With the Cas RAMP Module Complex to Cleave RNAs, Molecular Cell, (2012) vol. 45, Issue 3, 292-302.

The fusion proteins described herein can be used in place of or in addition to any of the Cas9 or Cpf1 proteins described in the foregoing references, or in combination with analogous mutations described therein, with a guide RNA appropriate for the selected Cas9 or Cpf1, i.e., with guide RNAs that target selected sequences.

In addition, the fusion proteins described herein can be used in place of the wild-type Cas9, Cpf1 or other Cas9 or Cpf1 mutations (such as the dCpf1 or Cpf1 nickase) as known in the art, e.g., a fusion protein with a heterologous functional domain as described in U.S. Pat. No. 8,993,233; US 20140186958; U.S. Pat. No. 9,023,649; WO/2014/099744; WO 2014/089290; WO2014/144592; WO144288; WO2014/204578; WO2014/152432; WO2115/099850; U.S. Pat. No. 8,697,359; US2010/0076057; US2011/0189776; US2011/0223638; US2013/0130248; WO/2008/108989; WO/2010/054108; WO/2012/164565; WO/2013/098244; WO/2013/176772; US20150050699; US 20150071899 and WO 2014/124284.

In some embodiments, the fusion proteins include a linker between the Cas9 pr Cpf1 variant and the heterologous functional domains. Linkers that can be used in these fusion proteins (or between fusion proteins in a concatenated structure) can include any sequence that does not interfere with the function of the fusion proteins. In preferred embodiments, the linkers are short, e.g., 2-20 amino acids, and are typically flexible (i.e., comprising amino acids with a high degree of freedom such as glycine, alanine, and serine). In some embodiments, the linker comprises one or more units consisting of GGGS (SEQ ID NO:5) or GGGGS (SEQ ID NO:6), e.g., two, three, four, or more repeats of the GGGS (SEQ ID NO:5) or GGGGS (SEQ ID NO:6) unit. Other linker sequences can also be used.

In some embodiments, the variant protein includes a cell-penetrating peptide sequence that facilitates delivery to the intracellular space, e.g., HIV-derived TAT peptide, penetratins, transportans, or hCT derived cell-penetrating peptides, see, e.g., Caron et al., (2001) Mol Ther. 3(3):310-8; Langel, Cell-Penetrating Peptides: Processes and Applications (CRC Press, Boca Raton Fla. 2002); El-Andaloussi et al., (2005) Curr Pharm Des. 11(28):3597-611; and Deshayes et al., (2005) Cell Mol Life Sci. 62(16):1839-49.

Cell penetrating peptides (CPPs) are short peptides that facilitate the movement of a wide range of biomolecules across the cell membrane into the cytoplasm or other organelles, e.g. the mitochondria and the nucleus. Examples of molecules that can be delivered by CPPs include therapeutic drugs, plasmid DNA, oligonucleotides, siRNA, peptide-nucleic acid (PNA), proteins, peptides, nanoparticles, and liposomes. CPPs are generally 30 amino acids or less, are derived from naturally or non-naturally occurring protein or chimeric sequences, and contain either a high relative abundance of positively charged amino acids, e.g. lysine or arginine, or an alternating pattern of polar and non-polar amino acids. CPPs that are commonly used in the art include Tat (Frankel et al., (1988) Cell. 55:1189-1193, Vives et al., (1997) J Biol. Chem. 272:16010-16017), penetratin (Derossi et al., (1994) J. Biol. Chem. 269:10444-10450), polyarginine peptide sequences (Wender et al., (2000) Proc. Natl. Acad. Sci. USA 97:13003-13008, Futaki et al., (2001) J. Biol. Chem. 276:5836-5840), and transportan (Pooga et al., (1998) Nat. Biotechnol. 16:857-861).

CPPs can be linked with their cargo through covalent or non-covalent strategies. Methods for covalently joining a CPP and its cargo are known in the art, e.g. chemical cross-linking (Stetsenko et al., (2000) J. Org. Chem. 65:4900-4909, Gait et al. (2003) Cell. Mol. Life. Sci. 60:844-853) or cloning a fusion protein (Nagahara et al., (1998) Nat. Med. 4:1449-1453). Non-covalent coupling between the cargo and short amphipathic CPPs comprising polar and non-polar domains is established through electrostatic and hydrophobic interactions.

CPPs have been utilized in the art to deliver potentially therapeutic biomolecules into cells. Examples include cyclosporine linked to polyarginine for immunosuppression (Rothbard et al., (2000) Nature Medicine 6(11):1253-1257), siRNA against cyclin B1 linked to a CPP called MPG for inhibiting tumorigenesis (Crombez et al., (2007) Biochem Soc. Trans. 35:44-46), tumor suppressor p53 peptides linked to CPPs to reduce cancer cell growth (Takenobu et al., (2002) Mol. Cancer Ther. 1(12):1043-1049, Snyder et al., (2004) PLoS Biol. 2:E36), and dominant negative forms of Ras or phosphoinositol 3 kinase (PI3K) fused to Tat to treat asthma (Myou et al., (2003) J. Immunol. 171:4399-4405).

CPPs have been utilized in the art to transport contrast agents into cells for imaging and biosensing applications. For example, green fluorescent protein (GFP) attached to Tat has been used to label cancer cells (Shokolenko et al., (2005) DNA Repair 4(4):511-518). Tat conjugated to quantum dots have been used to successfully cross the blood-brain barrier for visualization of the rat brain (Santra et al., (2005) Chem. Commun. 3144-3146). CPPs have also been combined with magnetic resonance imaging techniques for cell imaging (Liu et al., (2006) Biochem. and Biophys. Res. Comm. 347(1):133-140). See also Ramsey and Flynn, Pharmacol Ther. 2015 Jul. 22. pii: S0163-7258(15)00141-2.

Alternatively or in addition, the variant proteins can include a nuclear localization sequence, e.g., SV40 large T antigen NLS (PKKKRRV (SEQ ID NO:7)) and nucleoplasmin NLS (KRPAATKKAGQAKKKK (SEQ ID NO:8)). Other NLSs are known in the art; see, e.g., Cokol et al., EMBO Rep. 2000 Nov. 15; 1(5): 411-415; Freitas and Cunha, Curr Genomics. 2009 December; 10(8): 550-557.

In some embodiments, the variants include a moiety that has a high affinity for a ligand, for example GST, FLAG or hexahistidine sequences. Such affinity tags can facilitate the purification of recombinant variant proteins.

For methods in which the variant proteins are delivered to cells, the proteins can be produced using any method known in the art, e.g., by in vitro translation, or expression in a suitable host cell from nucleic acid encoding the variant protein; a number of methods are known in the art for producing proteins. For example, the proteins can be produced in and purified from yeast, E. coli, insect cell lines, plants, transgenic animals, or cultured mammalian cells; see, e.g., Palomares et al., “Production of Recombinant Proteins: Challenges and Solutions,” Methods Mol Biol. 2004; 267:15-52. In addition, the variant proteins can be linked to a moiety that facilitates transfer into a cell, e.g., a lipid nanoparticle, optionally with a linker that is cleaved once the protein is inside the cell. See, e.g., LaFountaine et al., Int J Pharm. 2015 Aug. 13; 494(1):180-194.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

In some embodiments, the mutants have alanine in place of the wild type amino acid. In some embodiments, the mutants have any amino acid other than arginine or lysine (or the native amino acid).

### Expression Systems

In order to use the fusion proteins and guide RNAs described herein, it may be desirable to express them from a nucleic acid that encodes them. This can be performed in a variety of ways. For example, a nucleic acid encoding a guide RNA or fusion protein can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the fusion protein or for production of the fusion protein. The nucleic acid encoding the guide RNA or fusion protein can also be cloned into an expression vector, for administration to a plant cell, animal cell, preferably a mammalian cell or a human cell, fungal cell, bacterial cell, or protozoan cell.

To obtain expression, a sequence encoding a guide RNA or fusion protein is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of the nucleic acid depends on the particular application. For example, a strong constitutive promoter is typically used for expression and purification of fusion proteins. In contrast, when the fusion protein is to be administered in vivo for gene regulation, either a constitutive or an inducible promoter can be used, depending on the particular use of the fusion protein. In addition, a preferred promoter for administration of the fusion protein can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the fusion protein, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the fusion protein, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ. A preferred tag-fusion protein is the maltose binding protein (MBP). Such tag-fusion proteins can be used for purification of the engineered TALE repeat protein. Epitope tags can also be added to recombinant proteins to provide convenient methods of isolation, for monitoring expression, and for monitoring cellular and subcellular localization, e.g., c-myc or FLAG.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

The vectors for expressing the guide RNAs can include RNA Pol III promoters to drive expression of the guide RNAs, e.g., the H1, U6 or 7SK promoters. These human promoters allow for expression of gRNAs in mammalian cells following plasmid transfection. Alternatively, a T7 promoter may be used, e.g., for in vitro transcription, and the RNA can be transcribed in vitro and purified. Vectors suitable for the expression of short RNAs, e.g., siRNAs, shRNAs, or other small RNAs, can be used.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the fusion protein encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

In some embodiments, the fusion protein includes a nuclear localization domain which provides for the protein to be translocated to the nucleus. Several nuclear localization sequences (NLS) are known, and any suitable NLS can be used. For example, many NLSs have a plurality of basic amino acids, referred to as a bipartite basic repeats (reviewed in Garcia-Bustos et al, 1991, Biochim. Biophys. Acta, 1071:83-101). An NLS containing bipartite basic repeats can be placed in any portion of chimeric protein and results in the chimeric protein being localized inside the nucleus. In preferred embodiments a nuclear localization domain is incorporated into the final fusion protein, as the ultimate functions of the fusion proteins described herein will typically require the proteins to be localized in the nucleus. However, it may not be necessary to add a separate nuclear localization domain in cases where the DBD domain itself, or another functional domain within the final chimeric protein, has intrinsic nuclear translocation function.

The present invention also includes the vectors and cells comprising the vectors, and cells and transgenic animals expressing the fusion proteins.

### aTF Systems

Provided herein are aTF systems. The aTF systems described herein can include one or more aTF(s) and/or aTF components (e.g. programmable nucleic acid binding domains, gene expression modulating domains, fusion proteins, and RNAs), as described herein.

In some embodiments, the aTF systems described herein comprise aTF(s) targeting one or more enhancer regions. In some embodiments, the aTF systems described herein comprise aTF(s) targeting one or more promoter regions. In some embodiments, the aTF systems described herein comprise (i) one or more aTF(s) that target an enhancer region that interacts, e.g., upregulates, a promoter region and (ii) one or more aTF(s) that target the promoter region.

In some embodiments, the aTF system comprises one or more promoter-targeting aTF(s) and one or more enhancer-targeting aTF(s). In some embodiments, the promoter that the promoter-targeting aTF(s) targets and the enhancer that the enhancer-targeting aTF(s) targets modulate the expression of the same gene. In some embodiments, the promoter that the promoter-targeting aTF(s) targets and the enhancer that the enhancer-targeting aTF(s) targets modulate the expression of different gene(s). In some embodiments, the aTF system comprises one or more promoter-targeting aTF(s) and one or more enhancer-targeting aTF(s), wherein the promoter that the promoter-targeting aTF(s) targets and the enhancer that the enhancer-targeting aTF(s) targets modulate the expression of the same gene and one or more aTF(s) wherein the promoter that the promoter-targeting aTF(s) targets and the enhancer that the enhancer-targeting aTF(s) targets modulate the expression of different gene(s).

In some embodiments, the promoter-targeting aTF comprises: (i) a fusion protein comprising a nucleic acid sequence binding domain, e.g., a catalytically inactive Cas9 or Cpf1 variant, and a gene expression modulating domain, e.g., a gene activating domain, e.g., p65, VP40, VPR, or p300. In some embodiments, e.g., when the aTF is a CRISPR-based aTF, the promoter-targeting aTF further comprises one or more gRNA(s) targeted to a promoter sequence.

In some embodiments, the enhancer-targeting aTF comprises: (i) a fusion protein comprising a nucleic acid sequence binding domain, e.g., a catalytically inactive Cas9 or Cpf1 variant, and a gene expression modulating domain, e.g., a gene activating domain, e.g., p65, VP40, VPR, or p300. In some embodiments, e.g., when the aTF is a CRISPR-based aTF, the promoter-targeting aTF further comprises one or more gRNA(s) targeted to a enhancer sequence.

In some embodiments promoter-targeting aTF comprises (i) a fusion protein comprising a nucleic acid sequence binding domain, e.g., a catalytically inactive Cas9 or Cpf1 variant, and a first dimerizing domain, e.g., DmrA(s); and (ii) a fusion protein comprising a gene expression modulating domain, e.g., a gene activating domain, e.g., p65, VP40, VPR, or p300, and a second coupling domain, e.g., Dmr(C)s. In some embodiments, e.g., when the aTF is a CRISPR-based aTF, the promoter-targeting aTF further comprises one or more gRNA(s) targeted to a promoter sequence.

In some embodiments enhancer-targeting aTF comprises (i) a fusion protein comprising a nucleic acid sequence binding domain, e.g., a catalytically inactive Cas9 or Cpf1 variant, and a first dimerizing domain, e.g., DmrA(s); and (ii) a fusion protein comprising a gene expression modulating domain, e.g., a gene activating domain, e.g., p65, VP40, VPR, or p300, and a second coupling domain, e.g., Dmr(C)s. In some embodiments, e.g., when the aTF is a CRISPR-based aTF, the enhancer-targeting aTF further comprises one or more gRNA(s) targeted to a promoter sequence.

In some embodiments, the aTF system further comprises a dimerizing agent.

Also provided herein are aTF systems comprising one or more expression vector(s) encoding the aTF(s) described herein. In some embodiments, the elements of the aTF(s) are encoded on the same nucleic acid vector. In some embodiments, some or all of the elements of the aTF(s) are encoded on different expression vectors.

In some embodiments, the system comprises a cell transformed with the nucleic acid vector(s) encoding the aTF(s) described herein. In some embodiments, the system comprises a cell expressing the aTF(s) described herein.

### Modulation of Gene Expression

Also provided herein are methods for modulating gene expression using the aTF systems described herein.

In some instances, the present disclosure relates to artificial transcription factor (aTF) systems that include two or more distinct aTFs that can be directed to bring gene expression modulating domains to both promoter regions and enhancer regions of genes different sequences on a nucleic acid (e.g., DNA) and methods for modulating (e.g., increasing or activating) expression of target genes using such aTF systems. In some instances, the aTF systems described herein include two or more distinct aTFs that can each bind specifically to one or more nucleic acid sequences of one or more enhancers and one or more nucleic acid sequences of one or promoters of one or more target genes to modulate (e.g., increase or activate) expression of the one or more target genes, e.g., as compared to wild-type expression. For example, the aTF systems described herein can be used to (1) heterotopically activate expression of one or more target genes that is otherwise not expressed (or not expressed beyond a certain threshold level) in a normal cell-type-specific context; (2) further increase expression (e.g., as compared to wild-type expression levels) of one or more target genes whose expression is already activated by one or more transcription factors (e.g., that are bound to promoters of the one or more target genes); (3) target activation of a gene in an allele-specific manner by specifically directing aTFs to enhancer regions, promoter regions, or both enhancer and promoter regions of a gene in an allele-specific manner. Such allele-specific activation can be achieved when the enhancer and/or the promoter contain sequences at the same genomic coordinates that are different between the two (or more) alleles.

Because a single enhancer can modulate the expression of multiple target genes, the expression of multiple target genes can be regulated by one or more aTFs targeting a single enhancer if an aTF is also recruited to the promoter of the target gene to be activated. In some instances, multiple enhancers can modulate the expression of a single target gene, thus a plurality of different aTFs targeting a plurality of enhancers can be used to modulate the expression of a single target gene. In such instances, using a plurality of aTFs targeting multiple enhancers can increase the expression of the target gene to a greater extent than when a single type of aTF targeting a single enhancer is used.

In some instances, the aTF systems described herein can include multiple aTFs that target a plurality of different sequences of a single enhancer or a single promoter. In such instances, using a plurality of aTFs targeting multiple sequences of a single enhancer or promoter can increase the expression of the target gene to a greater extent than when a single type of aTF targeting a single sequence of an enhancer or promoter is used.

### Variants/Identity

In certain instances, the present disclosure also encompasses fusion proteins and other aTF components (e.g., gRNAs) having amino acid sequences or nucleic acid sequences that share certain % homology (e.g., greater than 75%, greater than 80%, greater than 85%, greater than 90%, greater than 95%, greater than 97%, greater than 98%, or greater than 99%) to the examples provided in the present disclosure.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%). For purposes of the present compositions and methods, at least 80% of the full length of the sequence is aligned.

For purposes of the present application, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

In some embodiments, the variants or mutants have alanine in place of the wild type amino acid. In some embodiments, the variants or mutants have any amino acid other than arginine or lysine (or the native amino acid).

### Exemplary Embodiments

Provided herein is an artificial transcription factor (aTF) system comprising: (a) a first aTF comprising a target gene enhancer-binding domain and a first gene expression modulating domain; and a second aTF comprising a target gene promoter-binding domain and a second gene expression modulating domain.

Also provided herein is an artificial transcription factor (aTF) system including: a plurality of aTF including a gene expression modulating domain and a CRISPR-Cas domain; a first gRNA including a sequence complementary to a target gene enhancer sequence; and a second gRNA including a sequence complementary to a target gene promoter sequence.

In some embodiments, the target gene expression is heterotopically increased (e.g., as compared to wild-type expression) when the first aTF is bound to the target gene enhancer and the second aTF is bound to the target gene promoter.

In some embodiments, the target gene expression is increased by at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 60 fold, at least 70 fold, at least 80 fold, at least 90 fold, at least 100 fold, at least 150 fold, at least 200 fold, at least 300 fold, at least 350 fold, at least 400 fold, at least 450 fold, at least 500 fold, at least 600 fold, at least 700 fold, at least 800 fold, at least 900 fold, at least 1000 fold, at least 1100 fold, at least 1200 fold, at least 1300 fold, at least 1400 fold, at least 1500 fold, at least 1600 fold, at least 1700 fold, at least 1800 fold, at least 1900 fold, at least 2000 fold, at least 2500 fold, or at least 3000 fold, compared to normotopic target gene expression, as measured by mRNA expression.

In some embodiments, the target gene expression is increased when the first aTF is bound to the target gene enhancer and the second aTF is bound to the target gene promoter, as compared to when only the first aTF is bound to the target gene enhancer without the second aTF bound to the target gene promoter.

In some embodiments, the target gene expression is increased when the first aTF is bound to the target gene enhancer and the second aTF is bound to the target gene promoter, as compared to when only the second aTF is bound to the target gene promoter without the first aTF bound to the target gene enhancer.

In some embodiments, the target gene expression is increased by at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 60 fold, at least 70 fold, at least 80 fold, at least 90 fold, at least 100 fold, at least 150 fold, at least 200 fold, at least 300 fold, at least 350 fold, at least 400 fold, at least 450 fold, at least 500 fold, at least 600 fold, at least 700 fold, at least 800 fold, at least 900 fold, at least 1000 fold, at least 1100 fold, at least 1200 fold, at least 1300 fold, at least 1400 fold, at least 1500 fold, at least 1600 fold, at least 1700 fold, at least 1800 fold, at least 1900 fold, at least 2000 fold, at least 2500 fold, or at least 3000 fold, as compared to: (1) when only the first aTF is bound to the target gene enhancer without the second aTF bound to the target gene promoter; or (2) when only the second aTF is bound to the target gene promoter without the first aTF bound to the target gene enhancer, as measured by mRNA expression.

In some embodiments, the first aTF includes a plurality of first aTFs each including a distinct target gene enhancer-binding domain, and where the plurality of first aTFs include target gene enhancer-binding domains that are specific to: (a) a plurality of distinct target gene enhancers; or (b) a plurality of distinct sequences of the target gene enhancer.

In some embodiments, the target gene expression is increased compared to when less than all of the plurality of first aTFs is bound to the target gene enhancer.

In some embodiments, the second aTF includes a plurality of second aTFs each including a distinct target gene promoter-binding domain, and where the plurality of second aTFs include target gene promoter-binding domains that are specific to a plurality of distinct sequences of the target gene promoter.

In some embodiments, the target gene expression is increased compared to when less than all of the plurality of second aTFs is bound to the target gene promoter.

In some embodiments, the target gene includes a plurality of target genes under the control of a single enhancer, and where the second aTF includes a plurality of second aTFs each including a distinct target promoter-binding domain, and where the plurality of distinct target promoter-binding domains are specific to promoters of the plurality of distinct target genes.

In some embodiments, the target gene includes a plurality of target genes under the control of a plurality of enhancers, and where (i) the first aTF includes a plurality of first aTFs each including distinct target enhancer binding domains, where the distinct target enhancer binding domains are specific to the plurality of enhancers; and (ii) the second aTF includes a plurality of second aTFs each including a distinct target promoter-binding domain, and where the plurality of distinct target promoter-binding domains are specific to promoters of the plurality of distinct target genes.

In some embodiments, the target gene includes: a first allele including a first promoter and a first enhancer; and a second allele including a second promoter and a second enhancer, where the target gene enhancer-binding domain of the first aTF is capable of activating the first enhancer of the target gene with greater efficiency than the second enhancer of the target gene.

In some embodiments, the first enhancer or the second enhancer are at the same genomic coordinates but differ from one another in sequence.

In some embodiments, the sequence difference includes a single-nucleotide polymorphism (SNP), a deletion, or an insertion.

In some embodiments, the sequence difference includes a SNP, and where the SNP disrupts or creates a PAM sequence.

In some embodiments, the first promoter or the second promoter are at the same genomic coordinates but differ from one another in sequence.

In some embodiments, the sequence difference includes a single-nucleotide polymorphism (SNP), a deletion, or an insertion.

In some embodiments, the aTF system is capable of selectively increasing expression of the target gene on the first allele.

In some embodiments, the target gene includes a plurality of target genes that are under the control of a single enhancer sequence, and where the second aTF is capable of activating the promoter sequence of one or more of the plurality of target genes with greater efficiency as compared to the promoter sequences of the other target genes.

In some embodiments, the target gene promoter-binding domain and the target gene enhancer-binding domain each includes a CRISPR-Cas domain, a zinc-finger DNA binding domain, or a transcription activator-like (TAL) effector domain.

In some embodiments, the first aTF, the second aTF, or both the first aTF and the second aTF include a CRISPR-Cas domain.

In some embodiments, at least one of the CRISPR-Cas domain is a catalytically inactive Cas9 (dCas9) or a catalytically inactive Cas12a (dCpf1).

In some embodiments, the CRISPR-Cas domain further includes a gRNA, where the gRNA includes a sequence complementary to a sequence of the target gene enhancer or a sequence of the target gene promoter.

In some embodiments, the CRISPR-Cas domain further includes a first gRNA including a sequence complementary to a sequence of the target gene enhancer and a second gRNA including a sequence complementary to a sequence of the target gene promoter.

In some embodiments, the first gene expression modulating domain and the second gene expression modulating domain are the same.

In some embodiments, the first gene expression modulating domain and the second gene expression modulating domain are different.

In some embodiments, the gene expression modulating domain includes an activation domain of p65, VPR, VP64, or p300.

In some embodiments, the gene expression modulating domain includes: (1) a protein that can introduce or remove covalent modifications to histones or DNA; or (2) a protein that directly or indirectly recruits other proteins in the cell that in turn can modulate gene expression.

In some embodiments, the protein that can introduce or remove covalent modifications to histones or DNA includes LSD1 or TET1.

In some embodiments, the first aTF, the second aTF, or the both the first and the second aTF each includes two or more gene expression modulating domains.

In some embodiments, the two or more gene expression modulating domains are coupled to the aTF by an inducible dimerization system.

In some embodiments, the inducible dimerization system includes a DmrA, and a DmrC.

In some embodiments, the the aTF system described herein further including a drug that induces the activity of an aTF.

In some embodiments, the addition of an inducible drug causes the aTF system to increase expression of the target gene.

In some embodiments, the enhancer sequence is located upsteam of the transcription start site of the target gene.

In some embodiments, the enhancer sequence is located greater than 500 nucleotides, greater than 1000 nucleotides, greater than 1500 nucleotides, greater than 2000 nucleotides, greater than 3000 nucleotides, greater than 4000 nucleotides, greater than 5000 nucleotides, greater than 10,000 nucleotides, greater than 50,000 nucleotides, greater than 100,000 nucleotides, greater than 500,000 nucleotides, or greater than 1,000,000 nucleotides upsteam of the transcription start site of the target gene.

In some embodiments, the enhancer sequence is located downstream of the transcription start site of the target gene.

In some embodiments, the enhancer sequence is located greater than 500 nucleotides, greater than 1000 nucleotides, greater than 1500 nucleotides, greater than 2000 nucleotides, greater than 3000 nucleotides, greater than 4000 nucleotides, greater than 5000 nucleotides, greater than 10,000 nucleotides, greater than 50,000 nucleotides, greater than 100,000 nucleotides, greater than 500,000 nucleotides, or greater than 1,000,000 nucleotides downstream of the transcription start site of the target gene.

In some embodiments, the enhancer sequence is a known enhancer sequence.

In some embodiments, the enhancer sequence is a putative enhancer sequence.

In some embodiments, the putative enhancer sequence includes DNase hypersensitivity sites (DHSs).

In some embodiments, the putative enhancer sequence is determined by chromosome conformation capture assay, circularized chromosome conformation capture assay, or Hi-C assay.

In some embodiments, the promoter sequence is located less than 1000 nucleotides upstream or less than 1000 nucleotides downstream of the transcription start site of the target gene.

In some embodiments, the promoter sequence is located less than 1000 nucleotides upstream of the transcription start site of the target gene.

In some embodiments, the target gene is the IL2RA gene, the MYOD1 gene, the CD69 gene, the HEB gene, the HBG1/2 gene, the APOC3 gene, or the HBB gene.

In some embodiments, the target gene is the APOA4 gene.

Provided herein are vectors including sequences encoding one or more of the components of an aTF system described herein.

Also provided herein are pharmaceutical compositions including an aTF system described herein or a vector described herein, and an acceptable pharmaceutical excipient.

Also provided herein are methods for increasing a target gene expression in a cell, the method including contacting the cell with an aTF system described herein, a vector described herein, or a pharmaceutical composition described herein, under condition sufficient to increase the target gene expression in the cell.

Also provided herein are methods for heterotopic activation of a target gene expression in a cell, the method including contacting the cell with an aTF system described herein, a vector described herein, or a pharmaceutical composition described herein, under condition sufficient to increase the target gene expression in the cell.

Also provided herein are methods for allele-specific activation of a target gene, the method including contacting a cell with an aTF system described herein, under condition sufficient to increase the target gene expression.

Also provided herein are methods for selective activation of one of a plurality of target genes under the control of an enhancer in a cell, the method including contacting the cell with an aTF system described herein under condition sufficient to increase the target gene expression.

In some embodiments, the cell is a eukaryotic cell. In some embodiments, the cell is a mammalian cell. In some embodiments, the cell is a human cell.

Also provided herein are methods for treating or preventing a condition or a disease in a subject, the method including administering to the subject an effective amount of a pharmaceutical composition described herein, thereby treating or preventing the condition or the disease.

Also provided herein are methods for treating or preventing a condition or a disease in a subject, the method including contacting an aTF system described herein, a vector described herein, or a pharmaceutical composition described herein, with a cell of the subject under condition sufficient to increase the target gene expression in the cell, thereby treating or preventing the condition or the disease in the subject.

In some embodiments, the condition or the disease is caused, at least in part, by insufficient expression of the target gene.

In some embodiments, the condition or the disease is caused, at least in part, by insufficient expression of the target gene on an allele.

In some embodiments, the condition or the disease is related to haploinsufficiency.

In some embodiments, the condition or the disease is caused, at least in part, by a dominant-negative gene.

In some embodiments, the administration of the pharmaceutical composition increases allele-specific expression of the target gene, thereby treating the condition or the disease.

In some embodiments, the condition or the disease is caused, at least in part, by insufficient expression of a target gene that is under the control of an enhancer, where the enhancer controls the expression of a plurality of genes.

In some embodiments, the aTF system described herein causes increase in the expression of the target gene in the cell or in the cell of the subject (e.g., as compared to wild-type expression) by at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 60 fold, at least 70 fold, at least 80 fold, at least 90 fold, at least 100 fold, at least 150 fold, at least 200 fold, at least 300 fold, at least 350 fold, at least 400 fold, at least 450 fold, at least 500 fold, at least 600 fold, at least 700 fold, at least 800 fold, at least 900 fold, at least 1000 fold, at least 1100 fold, at least 1200 fold, at least 1300 fold, at least 1400 fold, at least 1500 fold, at least 1600 fold, at least 1700 fold, at least 1800 fold, at least 1900 fold, at least 2000 fold, at least 2500 fold, or at least 3000 fold, as measured by mRNA expression.

Also provided herein are methods for identifying an enhancer of a target gene, the method including: contacting a cell with an aTF system described herein, where the target gene enhancer-binding domain of the first aTF is specific for a putative enhancer; comparing the target gene expression level of the cell with a threshold target gene expression level; and determining if the putative enhancer is an enhancer of a target gene by determining if the target gene expression level of the cell is greater than the threshold target gene expression level.

The methods and compositions disclosed herein provides several advantages. For example, in some instances, the aTF systems described herein can regulate the expression of target genes beyond the range that was possible using traditional aTFs that target enhancers or the promoter alone. This dynamic range of gene regulation provided by the aTF systems can also be adapted to regulate allele-selective activation of target genes, for example, by targeting sequences of the target gene enhancers or promoters that differ between the two alleles. Further, the aTF systems described herein can be used to selectively regulate the expression of multiple genes that are under the control of a single enhancer, or the expression of a gene that is under the control of multiple enhancers.

Yet another advantage is the highly programmable nature of the sequence specificity of the aTF systems provided herein, which can be useful, for example in screening multiple putative enhancer sequences of a target gene (e.g., by using a library of aTFs that specifically bind to putative enhancer sequences) to identify previously unknown enhancers a target gene.

### EXAMPLES

The invention is further described in the following examples, which do not limit the scope of the invention described in the claims.

The examples described herein show efficient heterotopic enhancer activation by CRISPR-SpCas9-based aTFs in human cells also requires concurrent activation of the target promoter and that doing so leads to a synergistic increase of target gene expression. The aTFs were used to achieve allele-selective activation of human gene expression by exploiting enhancer-embedded SNPs, to expand the dynamic range of human gene regulation mediated by aTFs, and to recapitulate in non-erythroid cells the stage-specific activation of different promoters in the human beta-globin gene cluster by a locus control region (LCR) enhancer. Our findings broaden the capabilities of the epigenetic editing toolbox by enabling robust heterotopic activation of enhancers to specific alleles and/or promoters as well as providing mechanistic insights into how enhancers normally function and choose their target promoters.

### Methods and Materials

The Methods and Materials described herein were used in the Examples provided herein.

Plasmids and Oligonucleotides

Diagram of the constructs and the list of plasmids and related sequences used in this study can be found below Table 6 and SpCas9 gRNA oligo sequences can be found in Table 7.

Human Cell Culture Conditions

HEK293 cells (Invitrogen) and U2OS cells (obtained from Dr. Toni Cathomen, University of Freiburg) were grown at 37° C., in 5% CO2 in Dulbecco's Modified Eagle Medium (DMEM) (ThermoFisher, cat #11995073) with 10% heat-inactivated fetal bovine serum (FBS) (ThermoFisher, cat #16140-089) and 1% penicillin and streptomycin (ThermoFisher, cat #1507006). HepG2 cells (ATCC, cat #HB-8065) were grown at 37° C., in 5% CO2 in Eagle's Minimum Essential Medium (EMEM) (ATCC, cat #30-2033) with 10% FBS and 1% penicillin and streptomycin. K562 cells (ATCC) were grown at 37° C., in 5% CO2 in Roswell Park Memorial Institute 1640 Medium (RPMI) (ThermoFisher, cat #62870-127) supplemented with 10% heat-inactivated FBS, 2 mM GlutaMax (ThermoFisher, cat #35050061), and 1% penicillin and streptomycin. Media supernatant was analyzed biweekly for any contamination of the cultures with mycoplasma using MycoAlert PLUS Mycoplasma Detection Kit (Lonza, cat #LT07-703).

Gene Activation Experiments

For direct fusion aTF experiments, HEK293, HepG2, U2OS and K562 cells were transfected with dCas9 activator plasmids (750 ng) and Cas9 gRNA plasmids (250 ng). For bi-partite aTF experiments, the cell lines were transfected with dCas9-DmrA(x4) plasmid (400 ng), DmrC-p65, DmrC-VP64 or DmrC-VPR plasmids (200 ng), and Cas9 gRNA plasmids (400 ng). 500 μM A/C heterodimerizer (Takara Clontech cat #635056) was added in the complete media at the time of transfection when bi-partite dCas9 activators were used. HEK293 and HepG2 cells were transfected using lipofection and U2OS and K562 were transfected by nucleofection. 24 hours prior to transfection, HEK293 cells (8.6×10⁴) and HepG2 cells (2.0×10⁵) were seeded in 12-well plates and then transfected with the plasmids using 3 μl of TransIT-293 (Minis Bio, cat #MIR2705) for HEK293 cells and 3 μl of TransfeX (ATCC, cat #ACS-4005) for HepG2 cells. U2OS cells and K562 cells (2×10⁵) were nucleofected with the plasmids using a 4D-Nucleofector (Lonza) and the DN-100 program with the SE Cell Line Nucleofector Kit and FF-120 program with the SF Cell Line Nucleofector Kit respectively. For gene activation analysis, total RNA was extracted from the cells 72 hours post-transfection using the NucleoSpin RNA Plus Kit (Clontech, cat #740984.250) and 50-250 ng of purified RNA was used for cDNA synthesis using a High Capacity RNA-to-cDNA kit (ThermoFisher, cat #4387406). For only the experiments at the β-globin, cDNA synthesis used the SuperScript III kit (ThermoFisher cat #18080-400) using oligo dT without random hexamers in the reverse transcription reaction. 3 μl of 1:4 to 1:20 diluted cDNA was amplified by quantitative PCR (qPCR) using Fast SYBR Green Master Mix (ThermoFisher, cat #4385612) with the primers listed elsewhere in this application. qPCR reactions were performed on a LightCycler 480 (Roche) with the following program: initial denaturation at 95° C. for 20 seconds (s) followed by 45 cycles of 95° C. for 3 s and 60° C. for 30 s. Ct values greater than 35 were considered as 35, because Ct values fluctuate for transcripts expressed at very low levels. Gene expression levels were normalized to HPRT1 and calculated relative to that of the negative controls (dCas9 activators and non-targeting gRNA plasmids).

Chromatin Immunoprecipitation (ChIP)

24 hours prior to transfections, HEK293 cells (2×10⁶) were seeded in 10 cm dishes and then transfected with 15 μg of plasmids (6 μg of dCas9-DmrA(x4), 3 μg of DmrC-p65, and 6μg of Cas9 gRNA) using 45 μl of TransIT-293. Cells were trypsinized 72 hours post-transfection, and ChIP experiments were performed using 5×10⁶ cells per sample per epitope. Chromatin from 1% formaldehyde-fixed cells were fragmented to 200-500 bp by sonication for 5-6 mins using the Branson Sonifier SFX250 (cat #101-063-965R) and immunoprecipitated with specific antibodies (details below) overnight at 4° C. Input DNA control samples were not treated with antibodies. Antibody-chromatin complexes were pulled down with protein G-Dynabeads (ThermoFisher, cat #10003D) for two hours, washed, eluted, and the cross-link reversed as previously described³⁷. After RNase A and proteinase K treatment, DNA was purified with paramagnetic beads as described previously (Rohland et al., “Cost-effective, high-throughput DNA sequencing libraries for multiplexed target capture,” Genome Res 22, 939-946, doi:10.1101/gr.128124.111 (2012)), and quantified using Qubit 4 Fluorometer (ThermoFisher, Cat#Q33226).

H3K27Ac ChIP-seq

H3K27Ac ChIP assay was conducted with 5 μg of H3K27Ac antibody (Active Motif, cat #39133) using the protocol described above. Sequencing libraries were prepared with 3 ng each of H3K27Ac ChIP DNA and input sample using SMARTer ThruPLEX DNA-seq kit (Takara, cat #R400675). Libraries were sequenced with single-end (SE) 75 cycles on an Illumina Nextseq 500 system at the Broad Institute of Harvard and MIT and the reads were aligned to human reference genome hg19 using Burrows-Wheeler Alignment (BWA) tool³⁹. Genome-wide coverage was calculated after extending to 200 bases (approximate fragment size) and averaged over 25 bp windows using igvtools (https://doi.org/10.1093/bib/bbs017). Coverage was then normalized and scaled using RSeqC (http://rseqc.sourceforge.net/#normalize-bigwig-py).

ChIP-qPCR

dCas9 fused to DmrA(x4) and p65 fused to DmrC were pulled down using 5 μg Cas9 antibody (Active motif, cat #61757) per ChIP assay as detailed above. The DNA was eluted in 30 μl of 10 mM Tris pH 7.5, and 3 μl of DNA was used for each qPCR using Fast SYBR Green Master Mix (ThermoFisher, cat #4385612) with the primers listed in Table 10. qPCR reactions were performed on a LightCycler 480 (Roche) with the following program: initial denaturation at 95° C. for 20 seconds (s) followed by 45 cycles of 95° C. for 3 s and 60° C. for 30 s. Relative enrichment for each target was calculated by normalization to input control.

RNA-Seq

RNA libraries were prepared from 500 ng of total RNA treated with Ribogold zero to remove ribosomal RNA, using TruSeq Stranded Total RNA Library Prep Gold kit (Illumina, cat #20020599) and TruSeq RNA Single Indexes. The RNA libraries were sequenced with SE 75 cycles on an Illumina Nextseq500 system at the Broad institute of Harvard and MIT. Reads were aligned to human reference genome hg19 using STAR (doi:10.1093/bioinformatics/bts635) and PCR duplicates were removed using Picard tools (http://broadinstitute.github.io/picard/). Reads aligning to ribosomal RNA were then filtered out of the alignment. Genomic coverage from filtered alignments were calculated by normalizing to sequencing depth using bedtools (https://doi.org/10.1093/bioinformatics/btq033). FPKMs were calculated using Cufflinks (https://doi.org/10.1038/nbt.1621).

ATAC-Seq

ATAC-seq libraries were constructed as previously described (Corces et al., “An improved ATAC-seq protocol reduces background and enables interrogation of frozen tissues,” Nat Methods 14, 959-962, doi:10.1038/nmeth.4396 (2017)). Cells (5×104) were incubated with DNase I (Worthington cat #LS002007) to remove DNA from dead cells, washed with PBS, resuspended in lysis buffer, and treated with transposase from Nextera DNA sample Prep Kit (Illumina, cat #FC-121-1030). After DNA purification, adaptor sequences were added to the tagmented DNA by PCR with the following program: 72° C. for 5 minutes (m), 98° C. for 30 s followed by 12 cycles of 98° C. for 10 s, 63° C. for 30 s and 72° C. for 1 m. DNA was purified with double-sided bead purification to remove primer dimers and large size (>1 kb) products. Purified products were sequenced with PE 150 cycles on an Illumina Nextseq500 system at the Broad institute of Harvard and MIT. Reads were aligned to human reference genome hg19 using BWA and filtered to exclude PCR duplicates and processed as previously described (Buenrostro et al., “Transposition of native chromatin for fast and sensitive epigenomic profiling of open chromatin, DNA-binding proteins and nucleosome position,” Nat Methods 10, 1213-1218, doi:10.1038/nmeth.2688 (2013)). Read start positions were shifted towards the 3′ end by 4 bp for reads aligning to plus strand and towards the 5′ end by 5 bp for reads aligning to minus strand. Genomic coverage was calculated by counting reads in 150 bp sliding windows at 20 bp steps across the genome and then normalized to 10 million reads in each experiment using bedtools (Quinlan et al., “BEDTools: a flexible suite of utilities for comparing genomic features,” Bioinformatics 26, 841-842, doi:10.1093/bioinformatics/btq033btq033 [pii] (2010)).

Defining APOC3 Enhancer Sequences for SNP Analysis

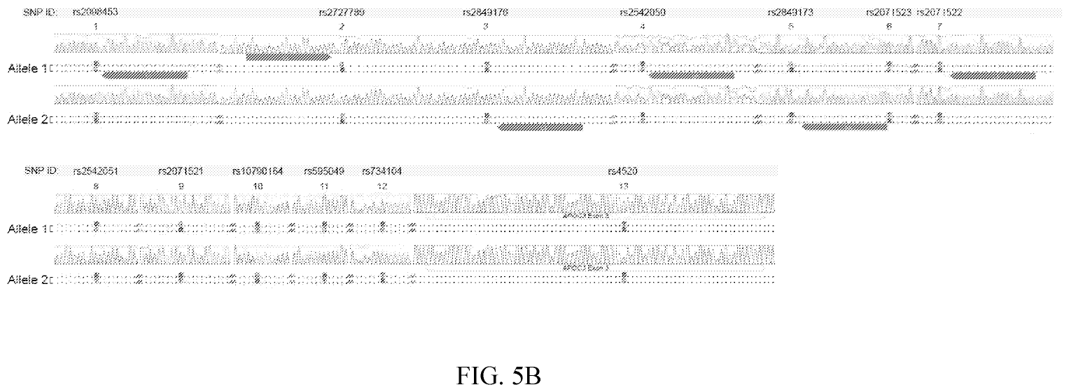

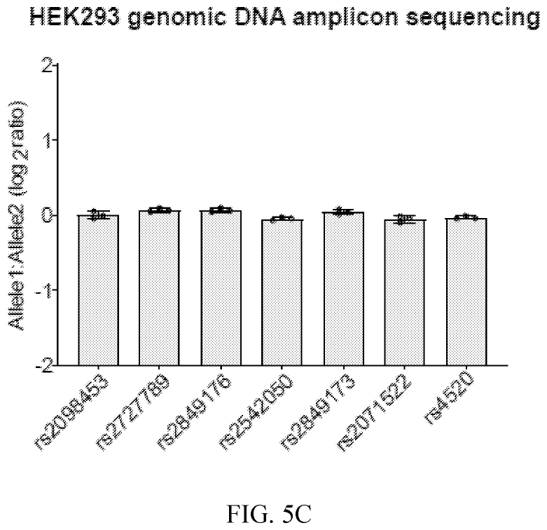

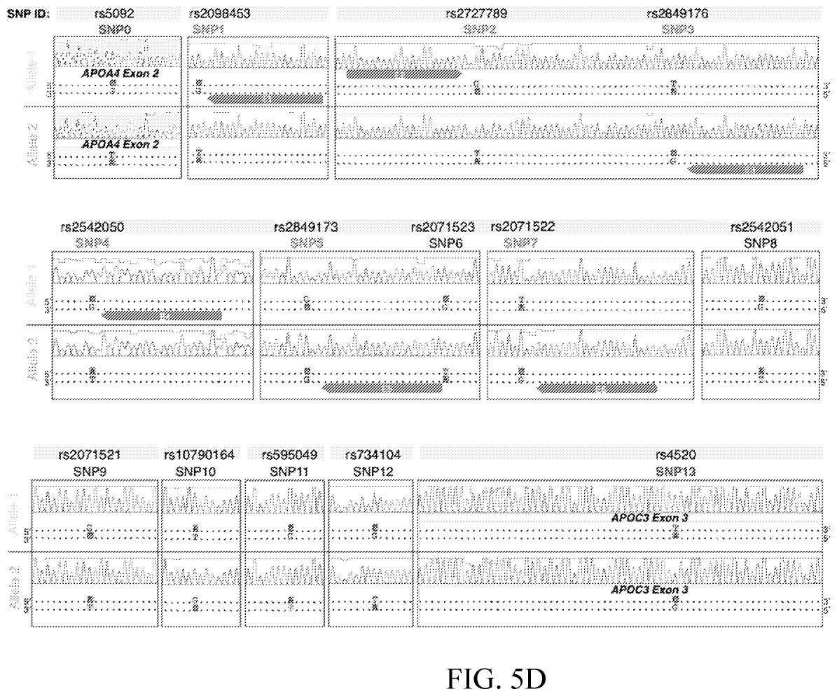

Known APOC3 enhancer sequences are located 500 to 890 bp upstream of the TSS10 (Zannis et al., “Transcriptional regulatory mechanisms of the human apolipoprotein genes in vitro and in vivo,” Curr Opin Lipidol 12, 181-207, doi:10.1097/00041433-200104000-00012 (2001)), and show open chromatin features in HepG2 cells in which APOC3 is highly expressed. We identified potential enhancer sequences in the region encompassing ˜4.4 Kb to 2 Kb upstream of TSS based on similar open chromatin features (FIG. 5).

Haplotype Analysis

Primers flanking the enhancer site E1 and APOC3 exon 3 SNP region (Table 11) were used to amplify ˜4.9 kb of HEK293 genomic DNA. Amplicons were cloned into topo vector using Zero Blunt TOPO PCR cloning kit (ThermoFisher, cat #450031) according to the blunt-end cloning kit protocol and ˜100 colonies were analyzed by Sanger sequencing.

Allele-Selective Binding of Activators and Gene Expression Experiments

Allele-selective binding of activators to gDNA identified by ChIP, allele ratio in native gDNA, and allele-selective gene expression were determined using next-generation sequencing. Libraries for amplicon sequencing were prepared in two steps by PCR. In the first step, target sites were amplified by PCR using primers that contain Ilumina adaptor sequences. The PCR reactions contained 50 ng of gDNA, 5 μl of ChIP DNA or 5 μl of 1:20 diluted cDNA, 500 nM each of forward and reverse primer, 200 μM dNTP, 1 unit of Phusion Hot Start Flex DNA Polymerase (NEB, Cat #M0535L) and 1× Phusion HF buffer in a total volume of 50 μl. The first PCR cycling conditions were 98° C. for 2 min followed by 25 cycles of 98° C. for 10 s, 65° C. for 12 s and 72° C. for 12 s, and a final 72° C. extension for 10 min. PCR products were purified using 0.7× to 1.2× paramagnetic beads according to amplicon size as described previously38 and quantified on Qubit 4 Fluorometer (ThermoFisher, Cat #Q33226) using 1× dsDNA high sensitivity kit (Cat #Q33231). Amplicons with Illumina adapters from the first PCR (1-19 ng) were barcoded with Illumina indexes containing sequences complementary to the adapter overhangs in a second PCR using the cycling conditions of 98° C. for 2 min, 7 cycles of 98° C. 10 s, 65° C. 30 s and 72° C. 30 s followed by 72° C. 10 min. The PCR products were purified as above and quantified by Qubit 4 Fluorometer. Amplicon libraries were sequenced paired-end (PE) 300 cycles on the Illumina Miseq using 300-cycle MiSeq Reagent Kit v2 (MS-102-2002) or Micro Kit v2 (Illumina, MS-103-2002). Demultiplexed FASTQ files were analyzed using TrimGalore (https://github.com/FelixKrueger/TrimGalore), FLASH2 (http://github.com/dstreett/FLASH2) and CRISPResso243. Allele-preferential expression of APOC3 gene in HEK293 was confirmed by RT-qPCR using allele-specific primers targeting APOC3 exonic SNP (rs4520) designed as per Li et. al. for mismatch amplification mutation assays (Li et al., “Genotyping with TaqMAMA,” Genomics 83, 311-320, doi:10.1016/j.ygeno.2003.08.005 (2004)). All the primers used in the above reactions are described herein. The specificity of the allele-specific primers was verified using U2OS cDNA in which the variant allele is not present (FIGS. 5A-5C).

Comparison of SNP Densities at Cas9 PAM Sequences in Promoters and Putative Enhancers

For this analysis, promoters were defined as +/−500 bp from TSS, and putative enhancers were determined as DNase Hypersensitivity Sites (DHSs) excluding promoter sequences described above. NCBI refseq version GCF_000001405.25_GRC37.p13 was used for defining TSS, and 83 DHS tracks of different cells and tissues from ENCODE/Roadmap project (encodeproject.org) were combined for the analysis. All SNPs from 1000 genomes project phase 3 were used for the analysis (internationalgenome.org/data) SNP sites were classified into three distinct categories based on their activity on the PAM sites: PAM creation, PAM disruption and Mixed (i.e. creation and disruption at the same time but on different strands). Based on the overlapping counts of SNPs in promoters and putative enhancers, we defined the SNP density as the number of SNPs in each region divided by the length of each regulatory element. Enhancer SNP density indicates the number of SNPs in each DHS divided by the peak size of each DHS. Promoter SNP density means the number of SNPs in each promoter divided by 1000 bp.

Statistical Analysis

For gene expression analysis, student t-test (two-tailed test assuming equal variance) was used and if the p-value is less than 0.05, the results were considered as statistically significant. To compare SNP densities between promoter and enhancer, Mann-Whitney U test was used, and if the p-value is less than 0.05, the results were considered as statistically significant.

Data and Code Availability

Data sets from amplicon sequencing have been deposited with the National Center for Biotechnology Information Sequence Read Archive ncbi.nlm.nih.gov/sra/PRJNA578485. Data sets from ChIP-seq, RNA-seq, and ATAC-seq experiments have been deposited with the Gene Expression Omnibus (GEO) repository with the accession number GSE 139190.

### Example 1

### Heterotopic Activation of Enhancer Sequences by Cas9-Based aTFs in Multiple Human Cell Lines

First, we assessed whether simple recruitment of an aTF could heterotopically activate enhancer sequences in human cells in which they are not normally active (FIG. 1A). We did this for three endogenous genes (IL2RA, CD69 and MYOD1) that are not expressed at detectable levels as measured by RNA-seq (FPKM values<1; see Methods and Materials) in four human cell lines: U2OS, HEK293, HepG2, and K562 (with the exception of CD69 which is expressed in K562 cells) (TABLE 4).

TABLE 4
RNA expression levels in fragments per kilobase
of transcript per million reads mapped (FPKM).

| | U2OS | | HEK293 | | HepG2 | | K562 | |
|---|---|---|---|---|---|---|---|---|
| | Rep1 | Rep2 | Rep1 | Rep2 | Rep1 | Rep2 | Rep1 | Rep2 |
| IL2RA | 0.03 | 0.00 | 0.00 | 0.00 | 0.00 | 0.00 | 0.00 | 0.00 |
| CD69 | 0.00 | 0.00 | 0.00 | 0.00 | 0.00 | 0.00 | 24.53 | 3.45 |
| MyoD1 | 0.00 | 0.00 | 0.11 | 0.13 | 0.00 | 0.00 | 0.00 | 0.00 |
| HBB | 0.00 | 0.00 | 1.23 | 0.51 | 0.00 | 0.00 | 2.91 | 5.48 |
| HBG1 | 0.25 | 0.00 | 0.11 | 0.14 | 2.71 | 0.00 | 641.59 | 1378.12 |
| HBG2 | 0.50 | 0.25 | 0.11 | 0.14 | 2.96 | 0.00 | 11213.60 | 24677.50 |
| HBE1 | 4.13 | 2.75 | 0.14 | 0.00 | 0.91 | 0.68 | 93.52 | 101.24 |
| APOA1 | 0.39 | 0.89 | 1.07 | 0.15 | 109.67 | 126.09 | 0.00 | 0.40 |
| APOC3 | 0.00 | 0.00 | 0.26 | 0.00 | 73.46 | 77.96 | 0.00 | 0.00 |
| APOA4 | 0.00 | 0.00 | 0.00 | 0.00 | 6.08 | 4.39 | 0.00 | 0.00 |
| APOA5 | 0.00 | 0.00 | 0.00 | 0.00 | 20.77 | 23.30 | 0.00 | 0.00 |

Bolded numbers refer to FPKM < 2.

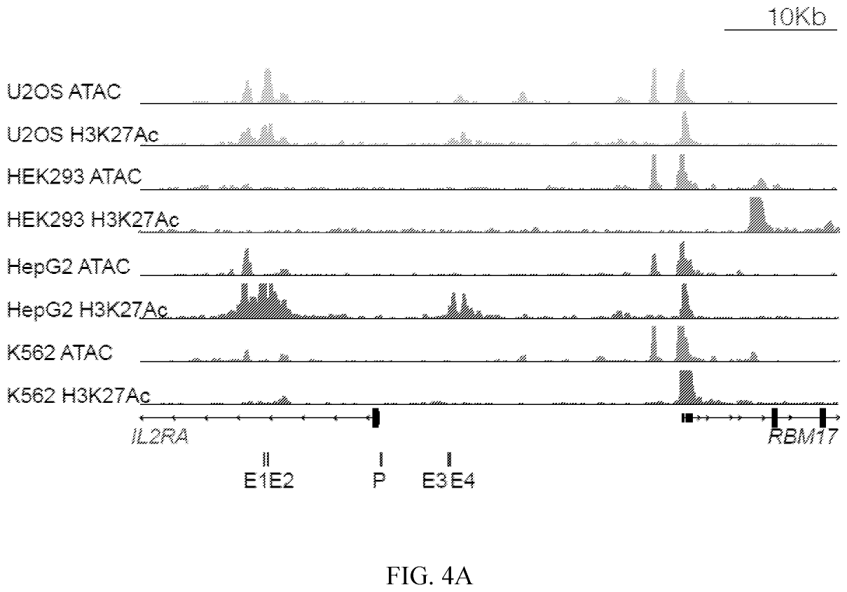

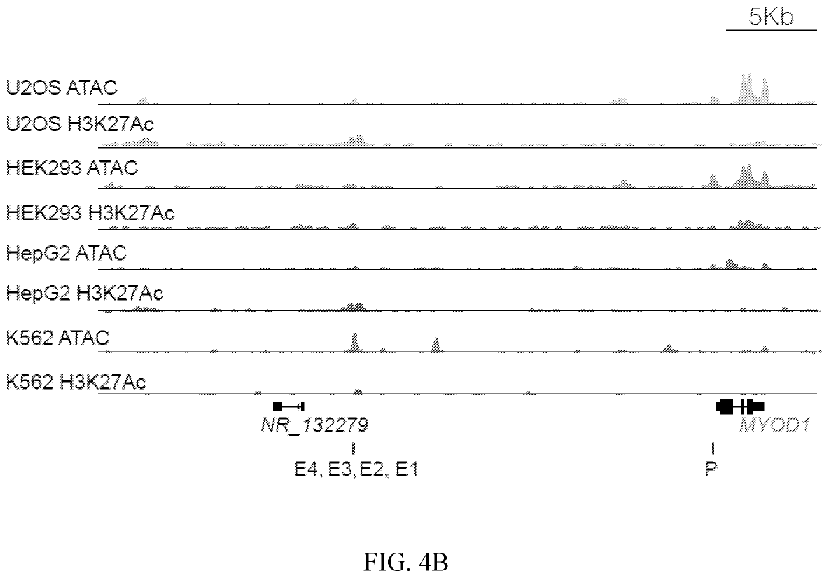

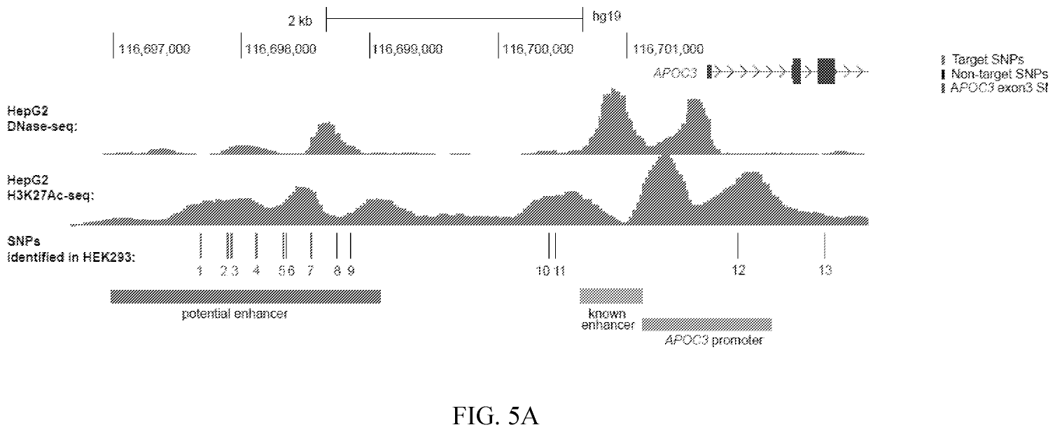

We used a bi-partite, small molecule-inducible, CRISPR-SpCas9 (hereafter referred to as Cas9)-based aTF that harbors a NF-KB p65 transcriptional activation domain⁶ (see FIG. 1B and Methods and Materials above). For the IL2RA gene, we designed guide RNAs (gRNAs) to target the bi-partite p65 aTF to sequences located ˜5 kb upstream or ˜10 kb downstream of the TSS (FIG. 1C) that were previously shown to be functional enhancers in T cells. See Simeonov et al., “Discovery of stimulation-responsive immune enhancers with CRISPR activation,” Nature 549, 111-115, doi:10.1038/nature23875 (2017). We tested gRNAs targeted to each of these two enhancer sites (FIG. 1C) for their abilities to stimulate transcription of the IL2RA gene when co-expressed with the bi-partite p65 aTF. We did not detect a significant increase in IL2RA gene transcription in any of the four human cell lines (FIG. 1C), regardless of whether the targeted enhancer sequences were in closed and inactive chromatin (HEK293 and K562 cells; FIG. 4A) or in open chromatin with H3K27Ac marks (U2OS and HepG2 cells; FIG. 4A). We also did not observe activation of the CD69 gene when we used gRNAs to target the bi-partite p65 aTF to an upstream conserved non-coding sequence 2 (CNS2) that has been previously shown to function as a stimulus-responsive enhancer in T-cells (Laguna et al., “New insights on the transcriptional regulation of CD69 gene through a potent enhancer located in the conserved non-coding sequence 2,” Mol Immunol 66, 171-179, doi:10.1016/j.molimm.2015.02.031 (2015)) (FIG. 1D). Additionally, testing of four different gRNAs that targeted the bi-partite p65 aTF to a core enhancer (CE) previously shown to activate the MYOD1 gene in myoblasts (located ˜20 kb upstream of the TSS (Chen et al., “The core enhancer is essential for proper timing of MyoD activation in limb buds and branchial arches,” Dev Biol 265, 502-512, doi:10.1016/j.ydbio.2003.09.018 (2004)) (FIG. 1E)) revealed only modest gene activation (five- to six-fold) with just one of the four gRNAs (E4) in HEK293 and U2OS cells and no significant activation with any of the four gRNAs in HepG2 and K562 cells (FIGS. 1E and 1H). Our results are consistent with and re-confirm earlier studies showing that simple recruitment of an aTF to an enhancer sequence is generally insufficient to induce efficient heterotopic activity.

The inability to consistently and efficiently induce heterotopic enhancer activation may be due to the closed state of the target gene promoter, rendering the enhancer unable to exert any activating effects (FIG. 1A). The MYOD1 promoter exhibited an open architecture and weak H3K27Ac marks in HEK293 and U2OS cells (FIG. 4B) in which we were able to weakly activate the MYOD1 CE enhancer heterotopically (FIGS. 1E and 1H); by contrast, the MYOD1 promoter remained closed in HepG2 and K562 cells (FIG. 4B) in which we could not heterotopically activate the CE enhancer (FIG. 1E).

Based on the above findings, we assessed whether concurrent activation of the target promoter with an aTF is required to enable heterotopic enhancer activation (FIG. 1A), by co-expressing each of the enhancer-targeted gRNAs described above with a promoter-targeted gRNA (FIGS. 1C-1E), thereby potentially recruiting the bi-partite p65 aTF to both enhancer and promoter sequences concurrently (FIG. 1A). We found that each of these promoter-targeted gRNAs on its own activated transcription of its associated target gene (ranges of 3- to 62-fold, 1- to 44-fold, and 2- to 52-fold for the IL2RA, CD69, and MYOD1, respectively) across the various cell lines tested (FIGS. 1C-1E). We also found that co-expression of enhancer- and promoter-targeted gRNAs with the bi-partite p65 activator led to synergistically higher levels of target gene transcription (i.e., greater levels of expression than what was observed with either gRNA individually) for most combinations of gRNAs (ranges of 5- to 224-fold, 6- to 160-fold, and 14- to 496-fold for the IL2RA, CD69, and MYOD1, respectively) (FIGS. 1C-1E). This represents as much as an additional 9-, 6-, and 31-fold upregulation in expression of IL2RA, CD69, and MYOD1 genes, respectively (FIGS. 1C-1E) due to heterotopic enhancer activation.

We explored the generality of this heterotopic enhancer activation strategy by testing a series of aTFs harboring different activation domains across four human cell lines. For these experiments, we assessed bi-partite activators harboring synthetic VP64 or VPR domains as well as direct fusions of dCas9 to p65, VPR, VP64 or the p300 domains (FIG. 1B) using the same pairs of enhancer-promoter gRNAs that we tested with the bi-partite p65 activator. We found that nearly all of these different aTFs were capable of inducing concerted enhancer-promoter activation at two or more of the gene promoters, albeit sometimes showing cell-type-specific efficiencies and lower activities than what was observed with the bi-partite p65 activator (FIGS. 1F-1H). Only the direct dCas9-p65 fusion failed to work efficiently in these experiments. Taken together, the findings described above show that efficient and robust heterotopic enhancer sequence activation can be achieved by recruiting aTFs to both the enhancer and target promoter simultaneously.

### Example 2

### Induction of Allele-Selective Gene Upregulation and Expansion of the Dynamic Range of Gene Expression in Human Cells Using Heterotopic Enhancer Activation

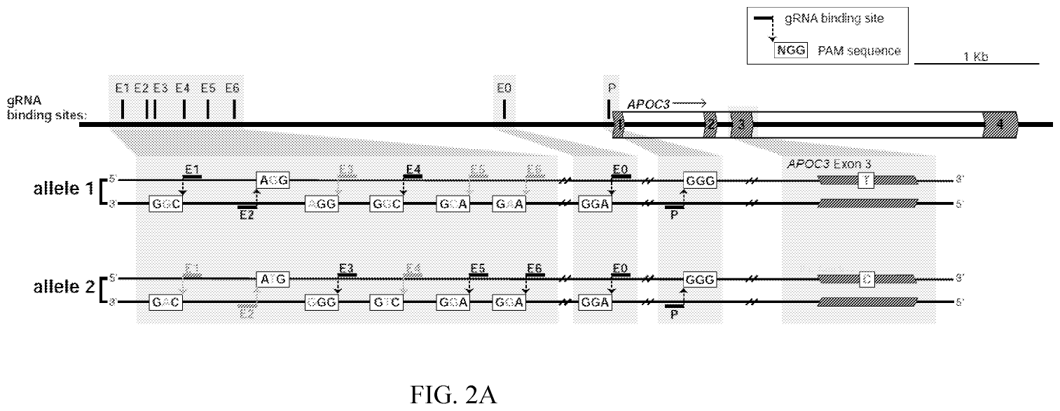

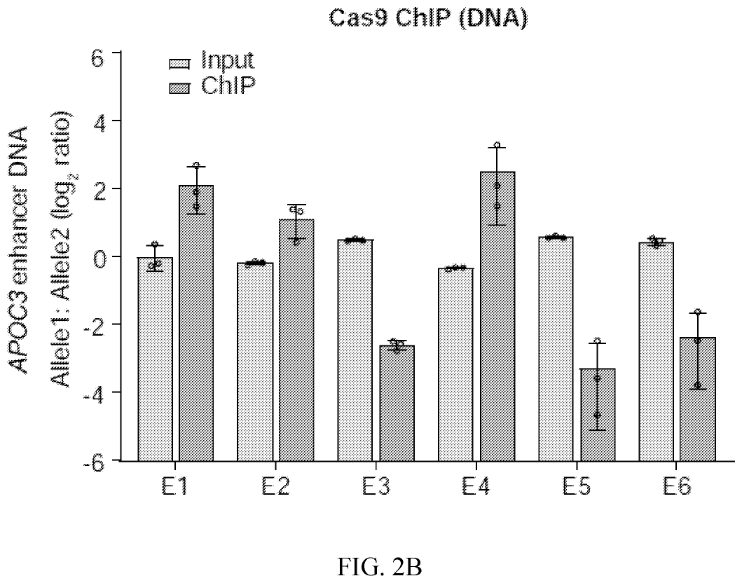

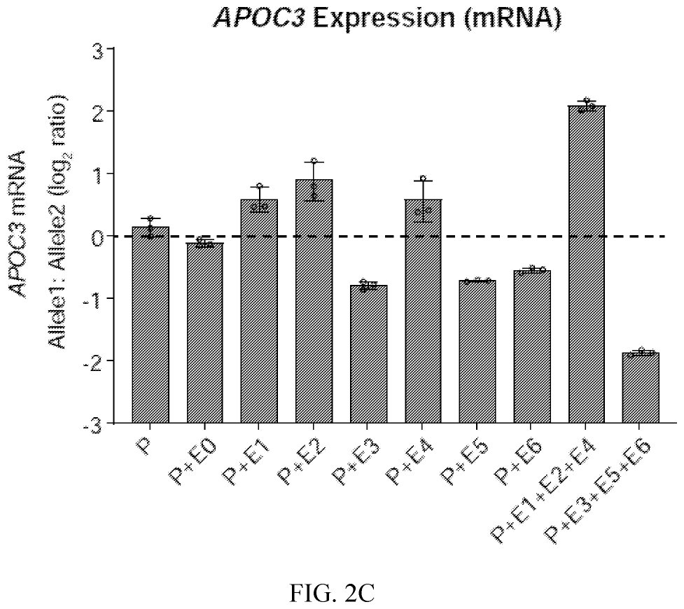

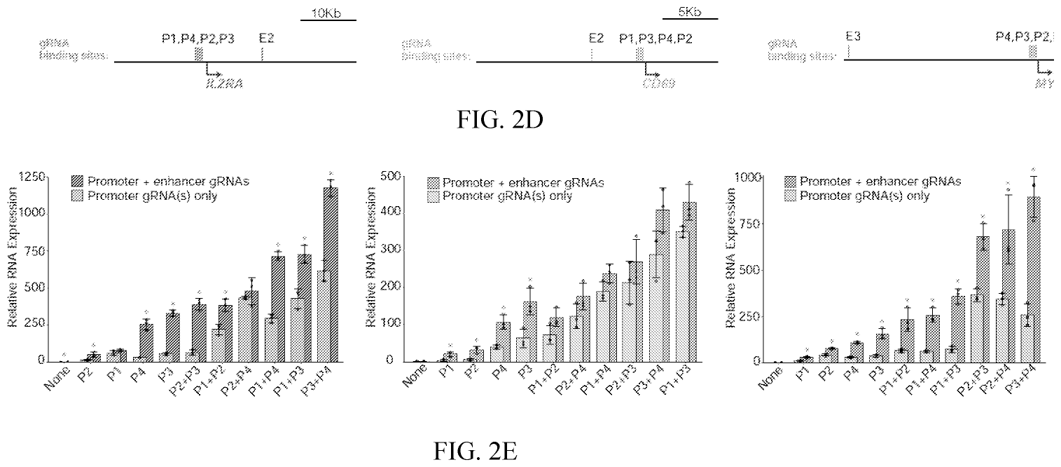

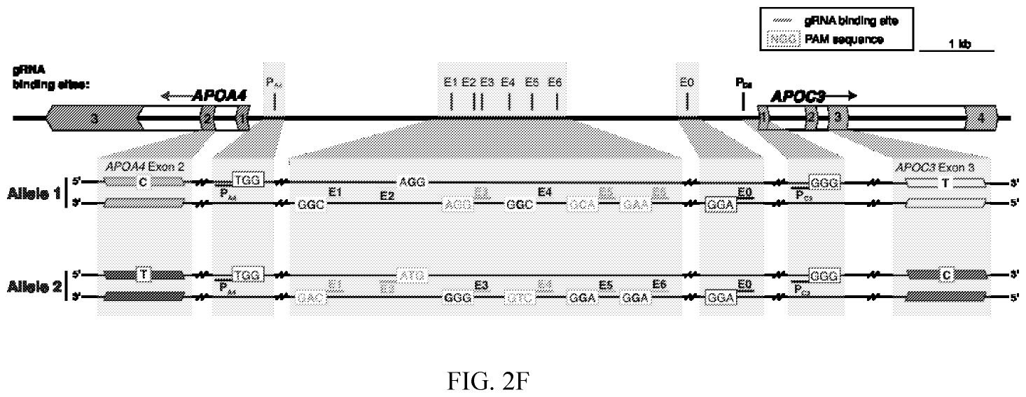

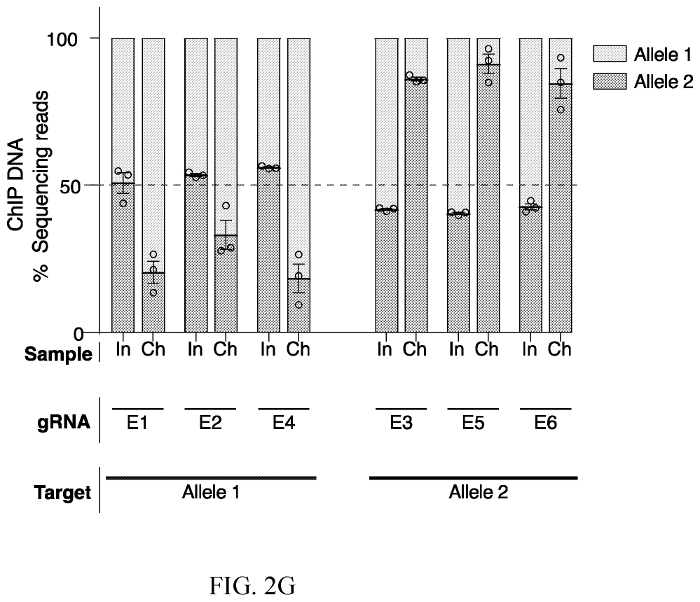

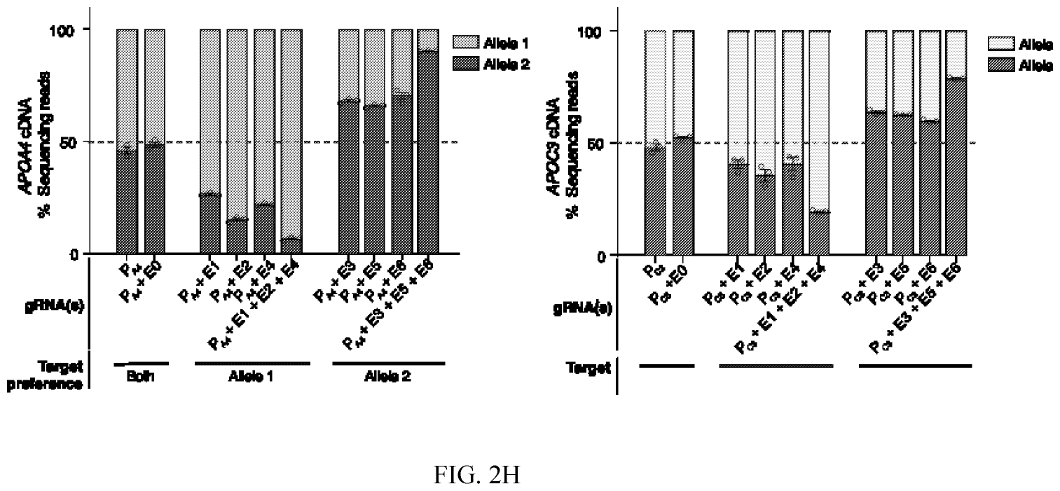

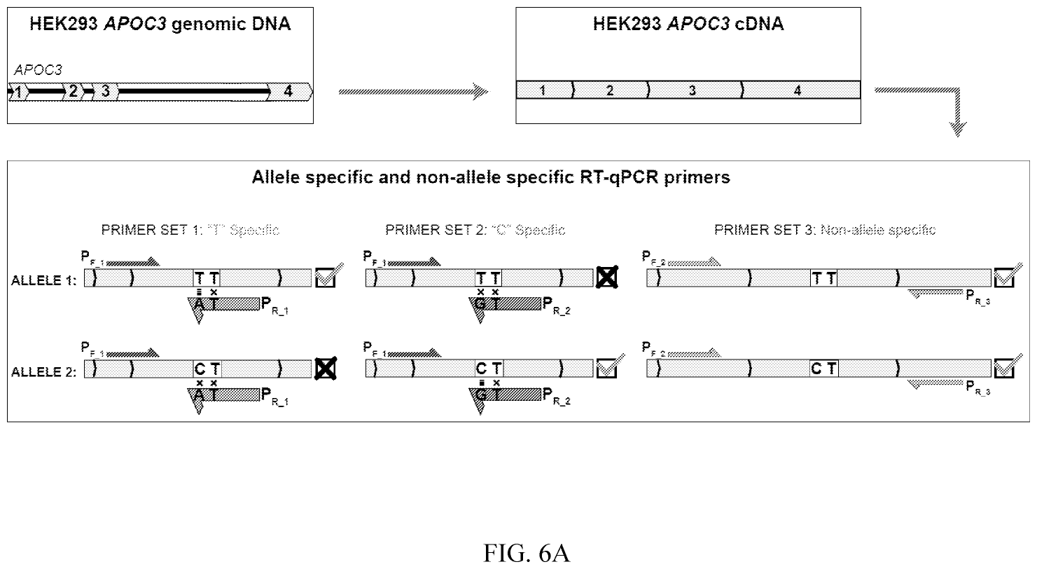

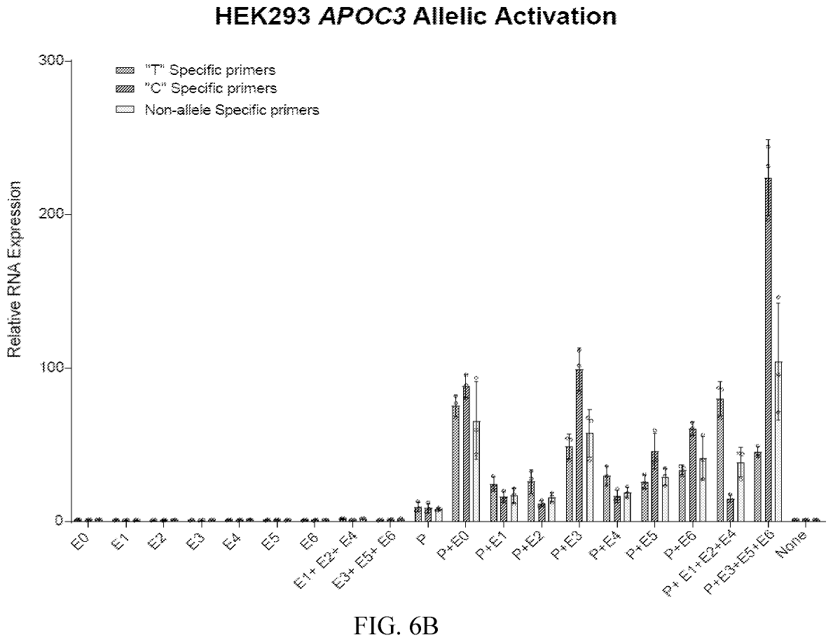

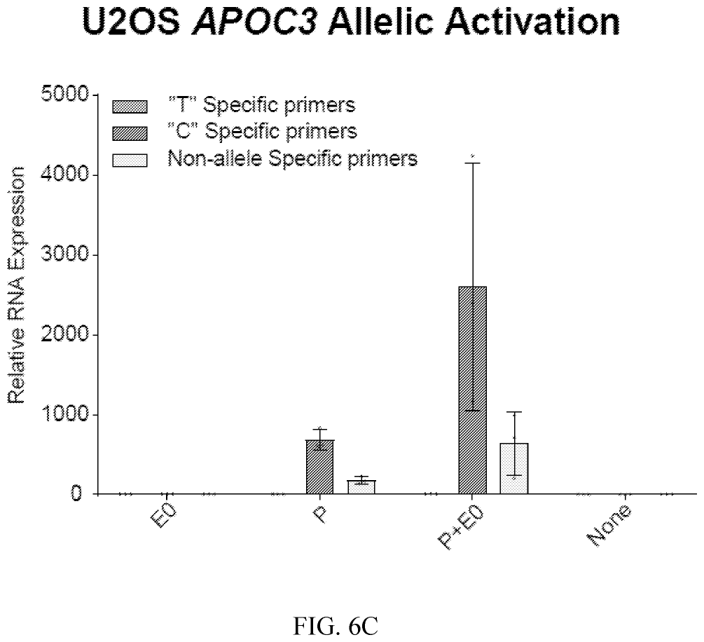

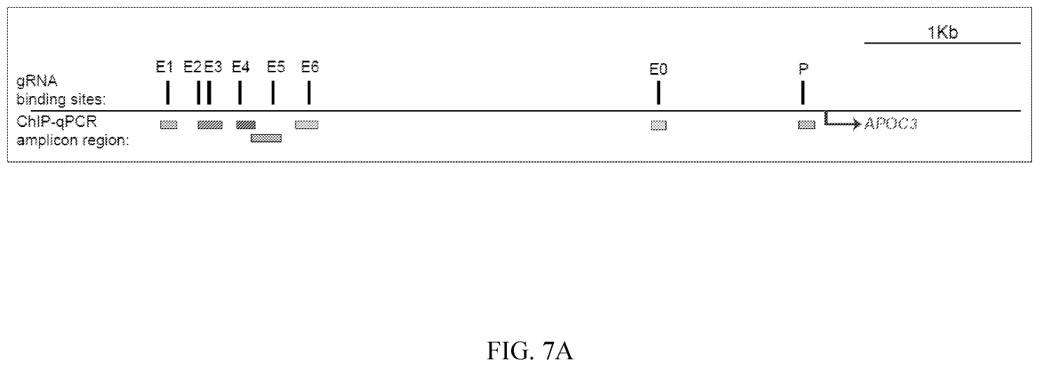

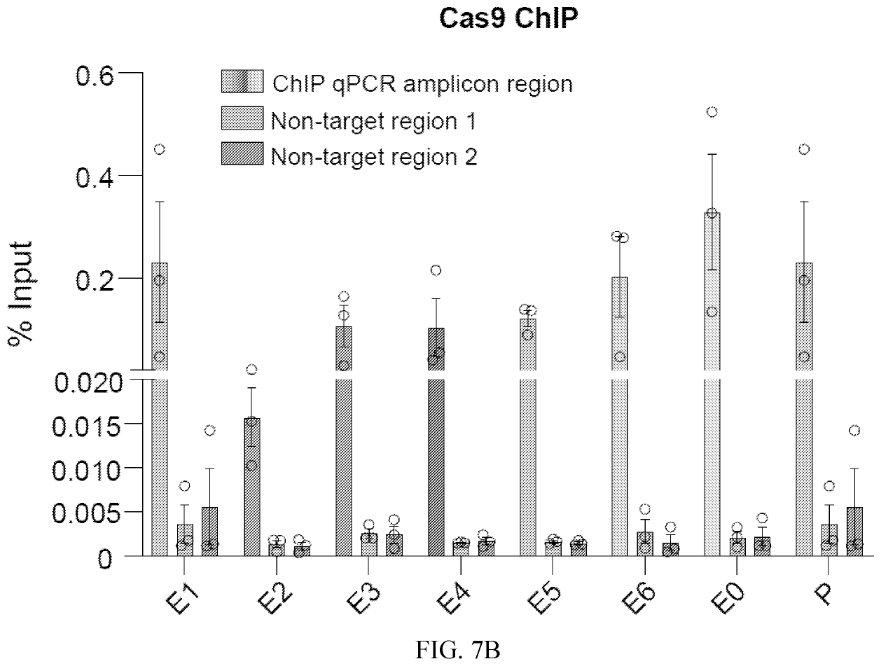

Having established our ability to use enhancer-bound aTFs for gene regulation, we assessed whether we could exploit DNA sequence variation in enhancer sequences to achieve allele-selective target gene activation. To do this, we sequenced a known enhancer (Ktistaki et al., “Transcriptional regulation of the apolipoprotein A-IV gene involves synergism between a proximal orphan receptor response element and a distant enhancer located in the upstream promoter region of the apolipoprotein C-III gene,” Nucleic Acids Res 22, 4689-4696, doi:10.1093/nar/22.22.4689 (1994); and Zannis et al., “Transcriptional regulatory mechanisms of the human apolipoprotein genes in vitro and in vivo,” Curr Opin Lipidol 12, 181-207, doi:10.1097/00041433-200104000-00012 (2001)) and coding sequences of the human APOC3 and APOA4 gene in HEK293 cells (Methods and Materials). This analysis identified a SNP in exon 3 of APOC3 and a SNP in exon 2 of APOA4 that distinguished two different alleles but no SNPs in the known enhancer (FIGS. 2A and 5). However, DNase-seq and H3K27Ac-seq data from the UCSC genome browser and our own analysis of HepG2 cells in which APOC3 is highly expressed (FIG. 5 and Table 4) identified additional regions just upstream of the known enhancer that exhibited features consistent with potential enhancers (i.e., H3K37Ac and ATAC-seq open chromatin peaks) and we identified 11 SNPs in these regions that differed between the two alleles (FIGS. 2A and 4). Using this information, we designed six enhancer gRNAs (E1-E6) targeting sites with a single base difference between the two alleles in their associated PAM sequences; we also designed a promoter gRNA (P) and an enhancer gRNA (E0) targeting common sequences present in both alleles (FIG. 2A). We reasoned that the E1-E6 gRNAs might each be used with a Cas9-based aTF to differentially activate one of the two alleles (FIGS. 2A, 2F and 5). Each of the seven enhancer-targeted gRNAs substantially up-regulated APOC3 gene expression with a bi-partite dCas9-based p65 activator only when used concurrently with the promoter gRNA (FIG. 6). However, sequencing as well as quantitation of DNA from ChIP-PCR experiments performed with a Cas9 antibody showed differential binding to the allele with the intact NGG PAM in the presence of the E1-E6 gRNAs (FIGS. 2B, 2G and 7). Consistent with this, cDNA sequencing of APOC3 and APOA4 transcripts from these experiments revealed significant allelic imbalances (judged by the SNP in exon 3; see Methods and Materials) with each of the E1-E6 gRNAs relative to the E0 gRNA (FIGS. 2C and 2H), a finding further supported by allele-specific quantitative RT-qPCR (FIG. 6). The magnitude of allele-preferential APOC3 and APOA4 activation could be further increased by simultaneous expression of multiple enhancer-targeted gRNAs against the same allele (FIGS. 2C and 2H).

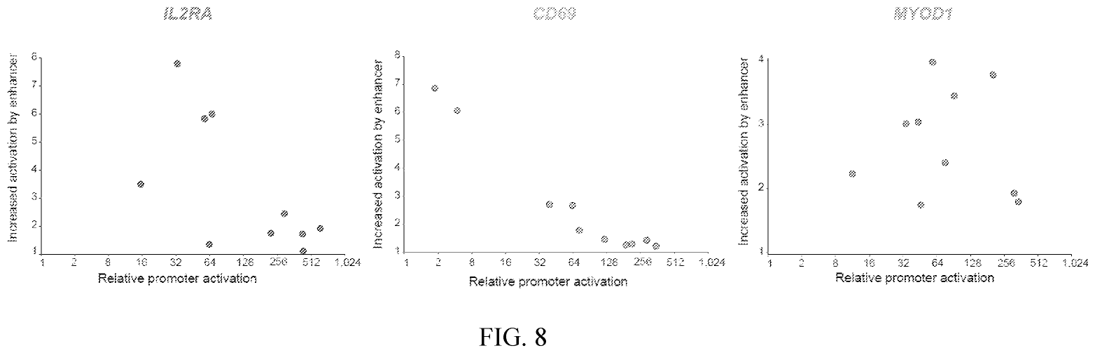

We next tested whether heterotopic enhancer activation can be used to further augment promoters that are already strongly activated by promoter-bound aTFs. Previous work has shown that targeting of more than one aTF to a promoter can yield synergistic increases in human gene transcription. See Hilton et al., “Epigenome editing by a CRISPR-Cas9-based acetyltransferase activates genes from promoters and enhancers,” Nat Biotechnol 33, 510-517, doi:10.1038/nbt.3199 (2015); Tak et al., “Inducible and multiplex gene regulation using CRISPR-Cpf1-based transcription factors,” Nat Methods 14, 1163-1166, doi:10.1038/nmeth.4483 (2017); Liu et al., “Regulation of an endogenous locus using a panel of designed zinc finger proteins targeted to accessible chromatin regions. Activation of vascular endothelial growth factor A,” J Biol Chem 276, 11323-11334, doi:10.1074/jbc.M011172200M011172200 [pii] (2001); Maeder et al., “Robust, synergistic regulation of human gene expression using TALE activators,” Nat Methods 10, 243-245, doi:10.1038/nmeth.2366nmeth.2366 [pii] (2013); Perez-Pinera et al., “RNA-guided gene activation by CRISPR-Cas9-based transcription factors,” Nat Methods 10, 973-976, doi:10.1038/nmeth.2600nmeth.2600 [pii] (2013); Perez-Pinera et al., “Synergistic and tunable human gene activation by combinations of synthetic transcription factors,” Nat Methods 10, 239-242, doi:10.1038/nmeth.2361nmeth.2361 [pii] (2013); Maeder, M. L. et al., “CRISPR RNA-guided activation of endogenous human genes,” Nat Methods 10, 977-979, doi:10.1038/nmeth.2598nmeth.2598 [pii] (2013); and Chavez et al., “Comparison of Cas9 activators in multiple species,” Nat Methods 13, 563-567, doi:10.1038/nmeth.3871 (2016). Consistent with this, we found that co-expression of various pairs of promoter-targeted gRNAs with the bi-partite p65 aTF led to greater than additive increases in gene transcription than what was observed with single gRNAs at the IL2RA, CD69, and MYOD1 genes (FIGS. 2D-2E). With various combinations of one or two promoter-bound aTFs, we observed mean activation ranges of 16- to 618-fold, 4- to 351-fold, and 11- to 365-fold for the IL2RA, CD69, and MYOD1 genes, respectively (FIG. 2E; light bars). Importantly, expression of a third gRNA targeted to an enhancer sequence generally led to even greater increases in gene transcription, expanding the mean activation values to as high as 1176-fold, 429-fold, and 894-fold for the IL2RA, CD69, and MYOD1 genes, respectively (FIG. 2E; dark bars). The impact of adding an enhancer-bound aTF was strongest for the IL2RA and MYOD1 genes but still measurable and significant for the CD69 gene (FIG. 2E); interestingly, for the IL2RA and CD69 genes, the magnitude of the enhancer-bound aTF effect on gene activation was inversely correlated with the magnitude of fold-activation induced by promoter-bound aTFs (FIG. 8).

### Example 3

### Directing of Heterotopic Enhancer Activities to a Specific Promoter in the Human β-Globin Locus Using dCas9-Based aTFs

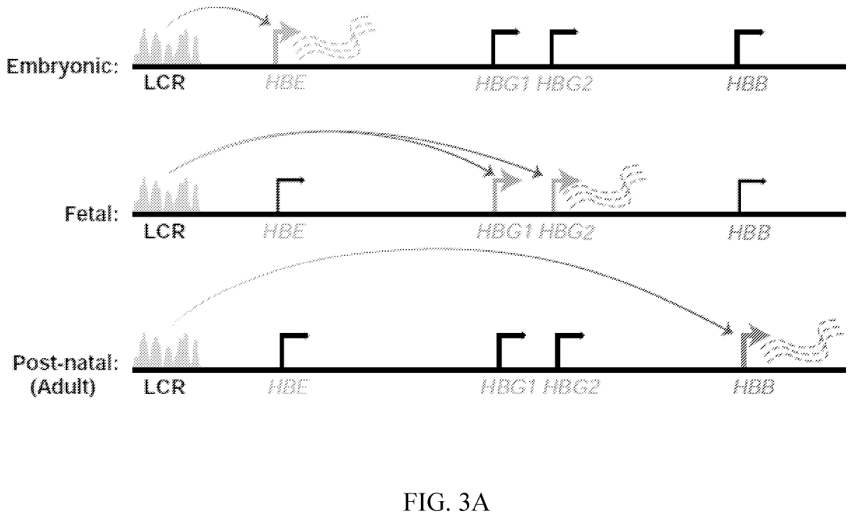

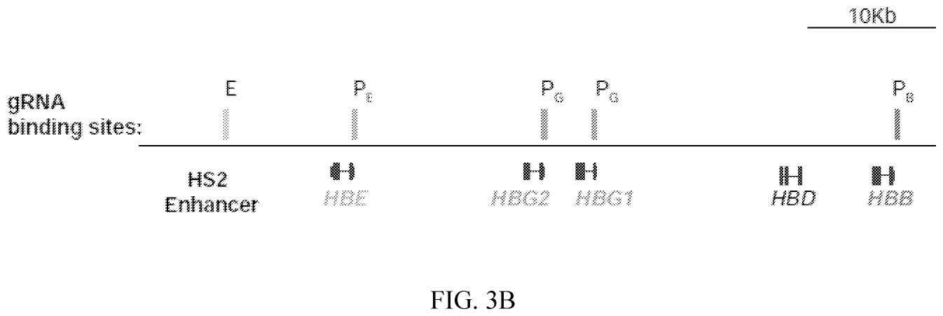

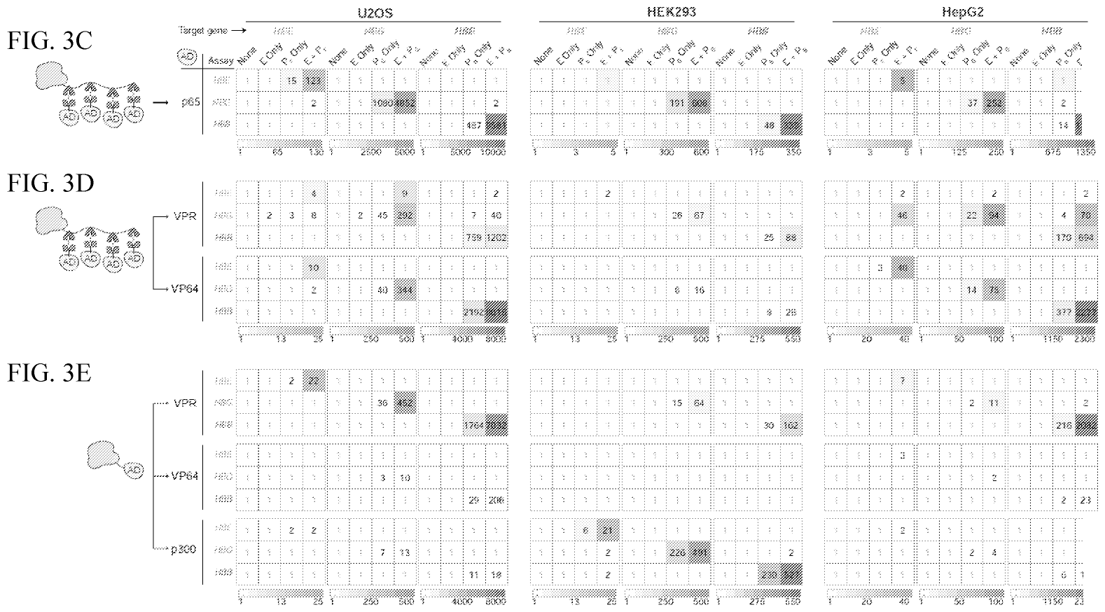

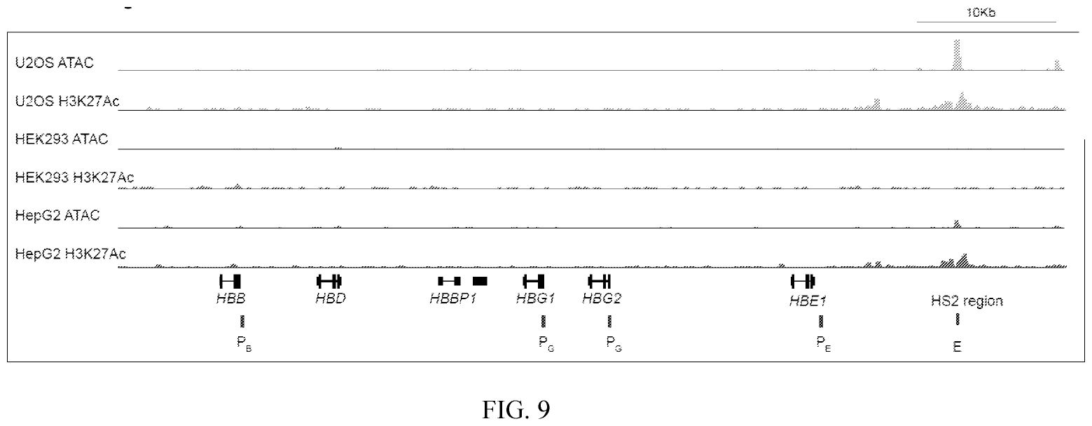

Here, we assessed whether the heterotopic enhancer activation strategy can be used to direct promoter choice for an enhancer that can potentially regulate multiple target genes. In erythroid cells, genes in the beta-globin cluster are preferentially expressed in a developmental stage-specific fashion by a distal locus control region (LCR) enhancer, leading to transcription from the HBE, HBG1/2, and HBB genes during embryonic, fetal, and post-natal stages of human development, respectively (Wienert et al., “Wake-up Sleepy Gene: Reactivating Fetal Globin for beta-Hemoglobinopathies,” Trends Genet 34, 927-940, doi:10.1016/j.tig.2018.09.004 (2018); Diepstraten et al., “Modelling human haemoglobin switching. Blood Rev 33, 11-23, doi:10.1016/j.blre.2018.06.001 (2019); Sankaran et al., “The switch from fetal to adult hemoglobin. Cold Spring Harb Perspect Med 3, a011643, doi:10.1101/cshperspect.a011643 (2013); and Sankaran et al., “Human fetal hemoglobin expression is regulated by the developmental stage-specific repressor BCL11A,” Science 322, 1839-1842, doi:10.1126/science.1165409 (2008).) (FIG. 3A). Using human cell lines (HEK293, HepG2 and U2OS) in which these three genes are not expressed at detectable levels (D), we tested whether we could direct the LCR enhancer to selectively turn on a particular target gene promoter in these cells using our concerted aTF approach. We co-expressed a gRNA targeted to the HBE, HBG1/2 or HBB promoter with the bi-partite p65 aTF and a gRNA designed to target the well-characterized DNase hypersensitive site 2 (HS2) site (Li et al., “Locus control regions: coming of age at a decade plus,” Trends Genet 15, 403-408, doi:10.1016/s0168-9525(99)01780-1 (1999).) within the LCR (FIG. 3B). Strikingly, in all three cell lines, we observed differential transcriptional activation of only the gene targeted by the promoter gRNA expressed (with the exception of the HBE gene that was not activated in HEK293 cells) and not the other two non-targeted genes (FIG. 3C). In each case, the level of gene activation we observed in the presence of both the LCR HS2 enhancer and promoter gRNAs was much higher than that observed in the presence of only the promoter gRNA (with the exception again of the HBE gene in HEK293 cells) (FIG. 3C). We were able to activate the LCR HS2 enhancer regardless of whether this region showed no, weak, or robust evidence of open chromatin (ATAC-seq) and H3K27Ac marks in HEK293, HepG2, or U2OS cells, respectively (FIG. 9). Targeting of the LCR enhancer alone using only the HS2-targeted gRNA was insufficient to upregulate transcription at any of the three promoters in the cell lines tested (FIG. 3C).

To assess the robustness of our strategy for directing the LCR enhancer to a desired target gene of interest, we tested this method using additional aTFs in the HEK293, U2OS, and HepG2 cell lines. For these experiments, we used bi-partite VPR or VP64 aTFs (FIG. 3D) and direct fusions of dCas9 to the p65, VPR or VP64 or p300 domains (FIG. 3E). We found that we could differentially direct efficient LCR enhancer activation to the HBE, HBG1/2, and HBB genes with bi-partite VPR or VP64 aTFs and with the direct dCas9-VPR aTF in all three cell lines (again with the exception of the HBE gene in HEK293 cells) as we observed with the bi-partite p65 activator (FIGS. 3D and 3E). Interestingly, the dCas9-p300 aTF also activated the LCR enhancer in all three cell types but was the most robust in HEK293 cells where, in contrast to all of the other aTFs tested, it could differentially activate expression of all three target promoters including that of the HBE gene (FIG. 3E). By contrast, heterotopic enhancer activation by dCas9-VP64 aTF was cell line-dependent, as it could differentially direct LCR activity robustly in U2OS cells and modestly in HepG2 cells but not at all in HEK293 cells (FIG. 3E). Lastly, the dCas9-p65 aTF failed to activate the LCR enhancer or any of the three gene promoters in the cell lines tested.

TABLE 5:
# of SNPs at NGG PAM sequences in regulatory elements

| | | | NGG PAM | |
|---|---|---|---|---|
| | NGG PAM | NGG PAM | creation + | |
| | creation | disruption | disruption | Sum |
| Promoter | 138,832 | 301,103 | 117,224 | 557,159 |
| (+/−500bp | | | | |
| TSS) | | | | |
| Putative | 1,788,668 | 3,547,400 | 1,107,015 | 6,443,083 |
| enhancer | | | | |
| Folds ratio (# | 12.88 | 11.78 | 9.44 | 11.56 |
| of SNPs in | | | | |
| putative | | | | |
| enhancer/ | | | | |
| promoter) | | | | |

### Exemplary aTF Constructs and Their Sequences

TABLE 6
Example aTF Constructs

| | Addgene | | SEQ |
|---|---|---|---|
| Name | # | Description | ID NO: |
| BPK1179 | TBD | pCAG-NLS-dSpCas9(D10A, H840A)- | 9 |
| | | NLS-3xFLAG-DmrA-DmrA-DmrA- | |
| | | DmrA | |
| BPK617 | TBD | pCAG-NLS-dSpCas9(D10A, H840A)- | 10 |
| | | NLS-3xFLAG-VP64 | |
| BPK1160 | TBD | pCAG-NLS-dSpCas9(D10A, H840A)- | 11 |
| | | NLS-3xFLAG-p65 | |
| JEH127 | TBD | pCAG-NLS-dSpCas9(D10A, H840A)- | 12 |
| | | NLS-3xHA-VPR(VP64-p65-RTA) | |
| BPK880 | TBD | BPK880: pCAG-DmrC-NLS-3xFLAG- | 13 |
| | | VP64 | |
| BPK1169 | 104564 | BPK1169: pCAG-DmrC-NLS- | 14 |
| | | 3xFLAG-p65 | |
| MMW948 | 104565 | MMW948: pCAG-DmrC-NLS- | 15 |
| | | 3xFLAG-VPR(VP64-p65-RTA) | |
| BPK1520 | 65777 | BPK1520 (pU6-BsmBICassette- | 16 |
| | | S. pyogenes.sgRNA) | |

### Sequences of Example aTF Constructs in Table 6:

BPK1179: pCAG-NLS-dSpCas9(D10A, H840A)-NLS-3xFLAG-DmrA-DmrA-DmrA-
DmrA
(SEQ ID NO: 9)
ATGGCGCCGAAAAAAAAACGCAAAGTGAACGGCGGAGGGTCCGGAGGAGGC

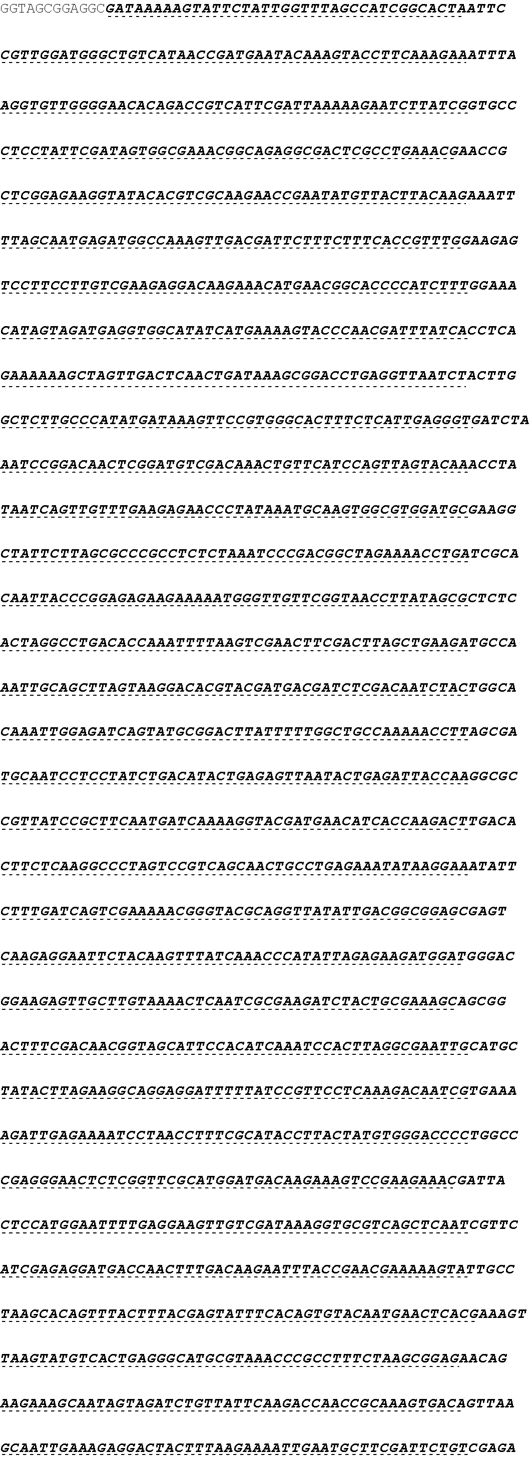

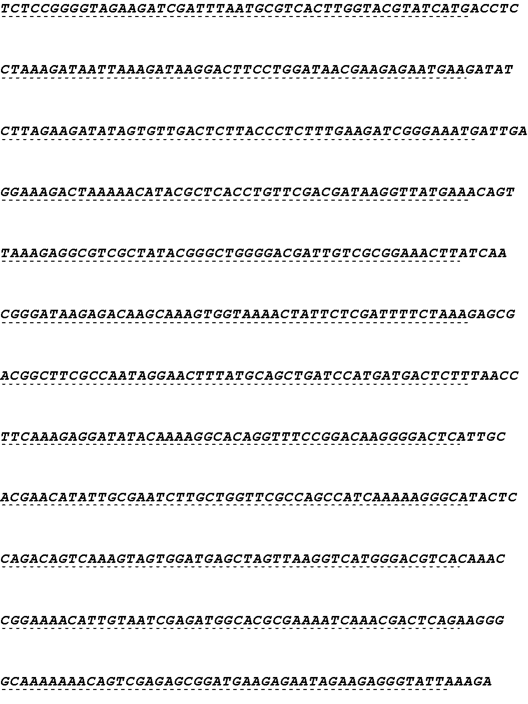

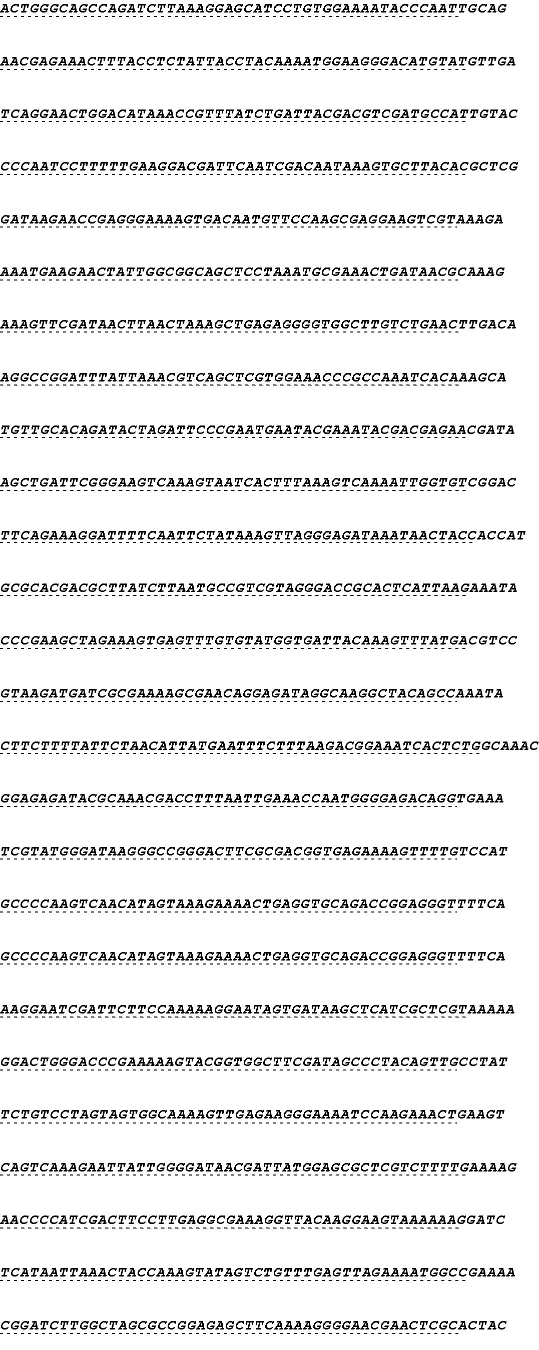

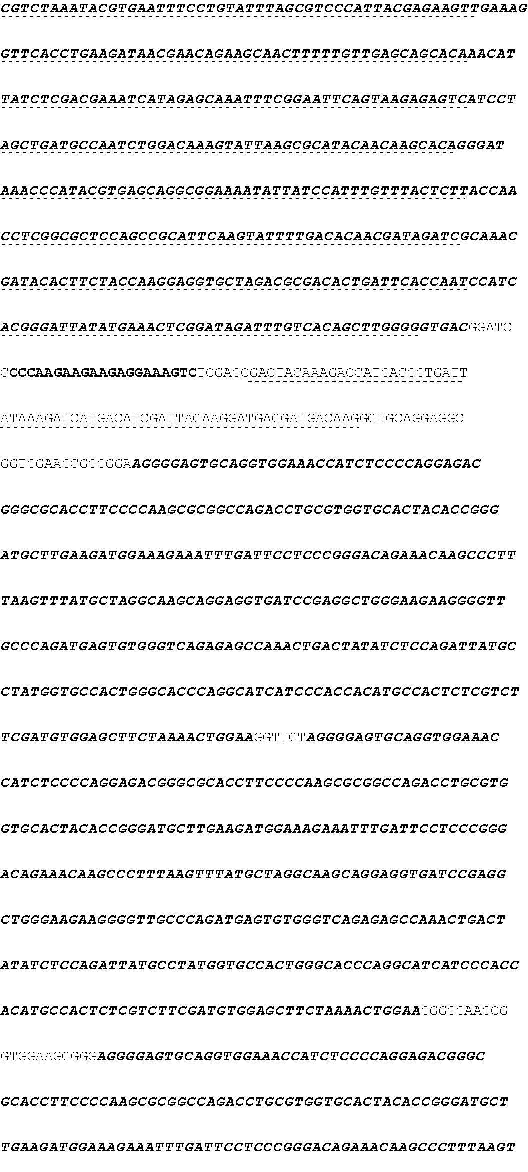

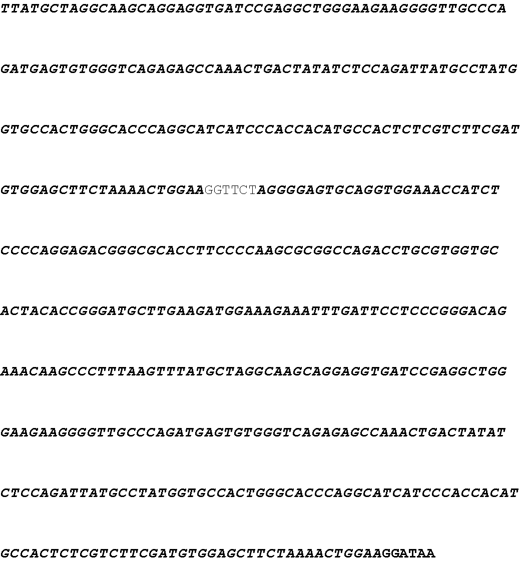

NNNN = NLS

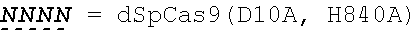

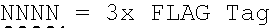

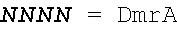

BPK617: pCAG-NLS-dSpCas9(D10A, H840A)-NLS-3xFLAG-VP64
(SEQ ID NO: 10)
ATGGCGCCGAAAAAAAAACGCAAAGTGAACGGCGGAGGGTCCGGAGGAGGC

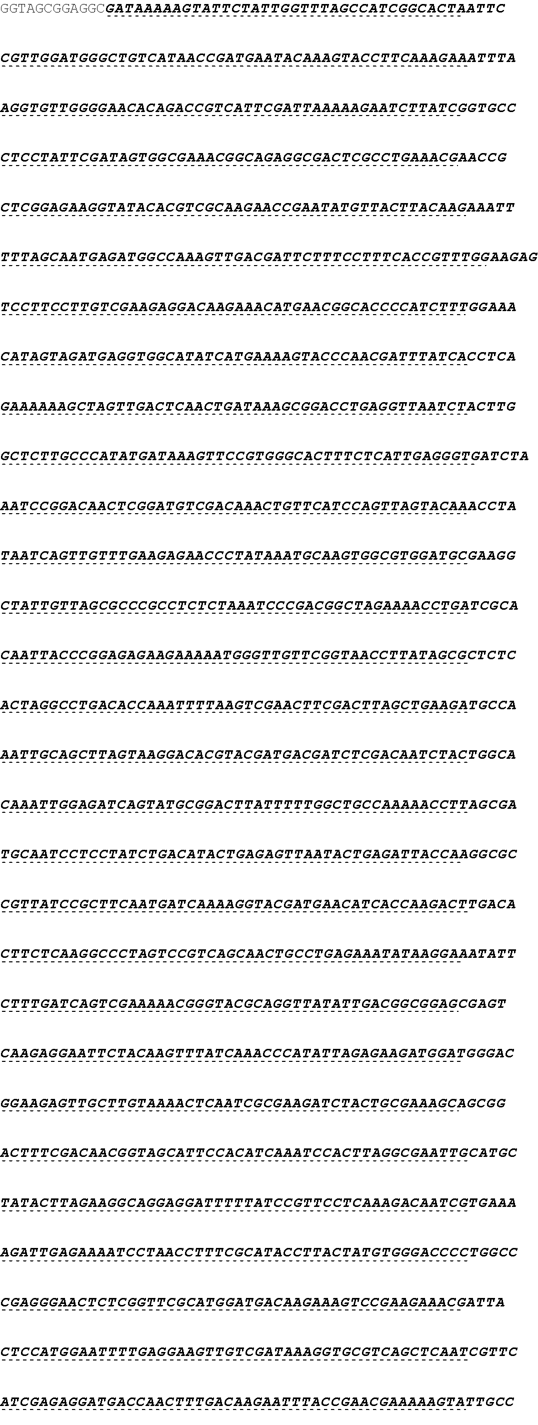

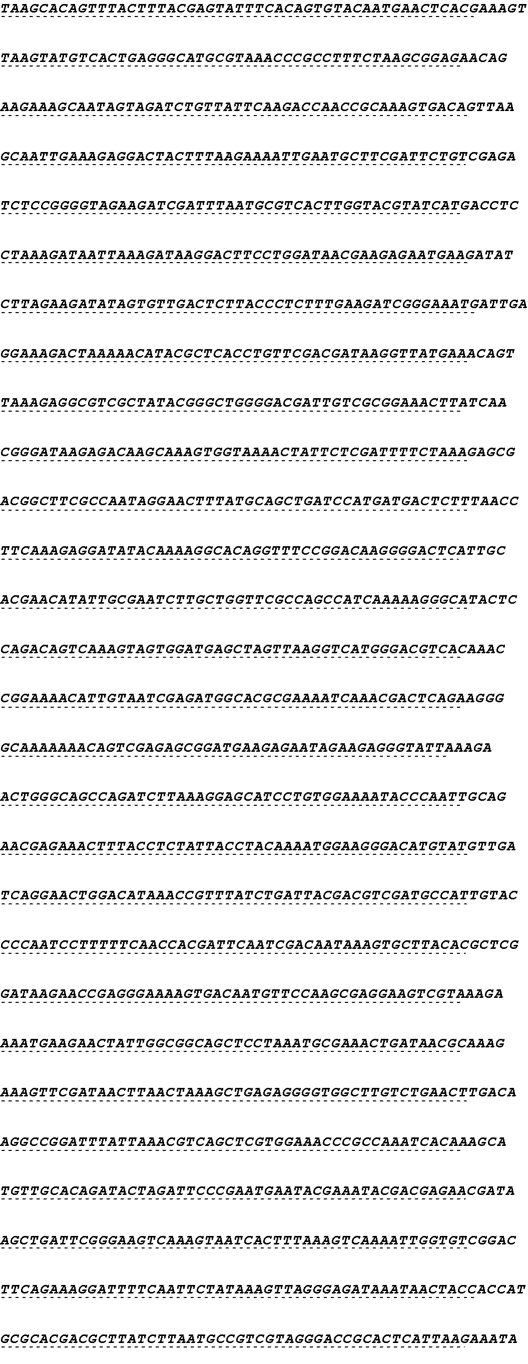

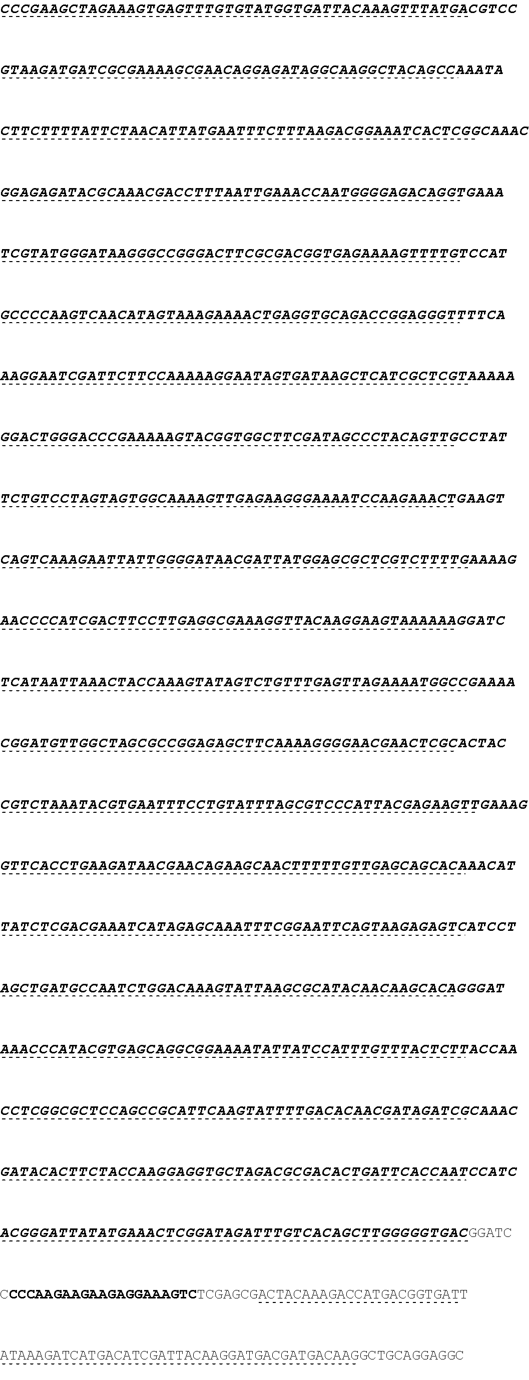

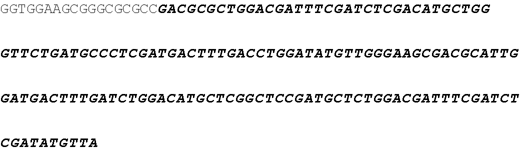

NNNN = NLS

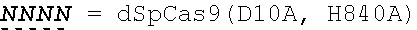

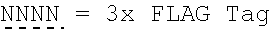

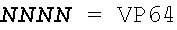

BPK1160: pCAG-NLS-dSpCas9(D10A, H840A)-NLS-3xFLAG-p65
(SEQ ID NO: 11)
ATGGCGCCGAAAAAAAAACGCAAAGTGAACGGCGGAGGGTCCGGAGGAGGC

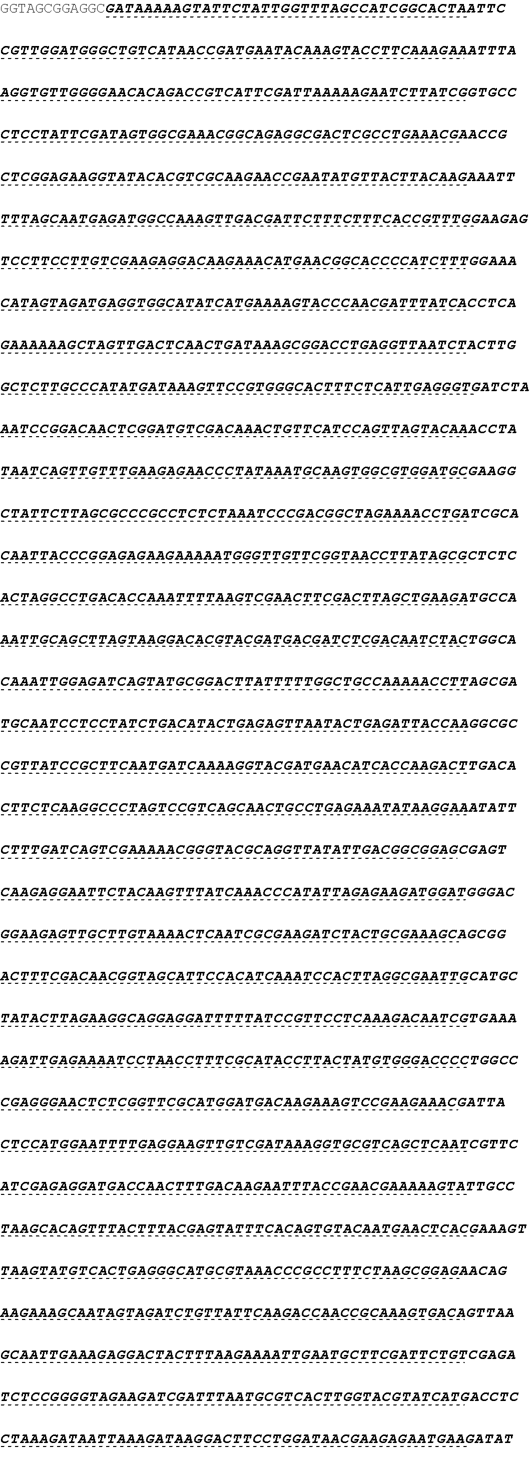

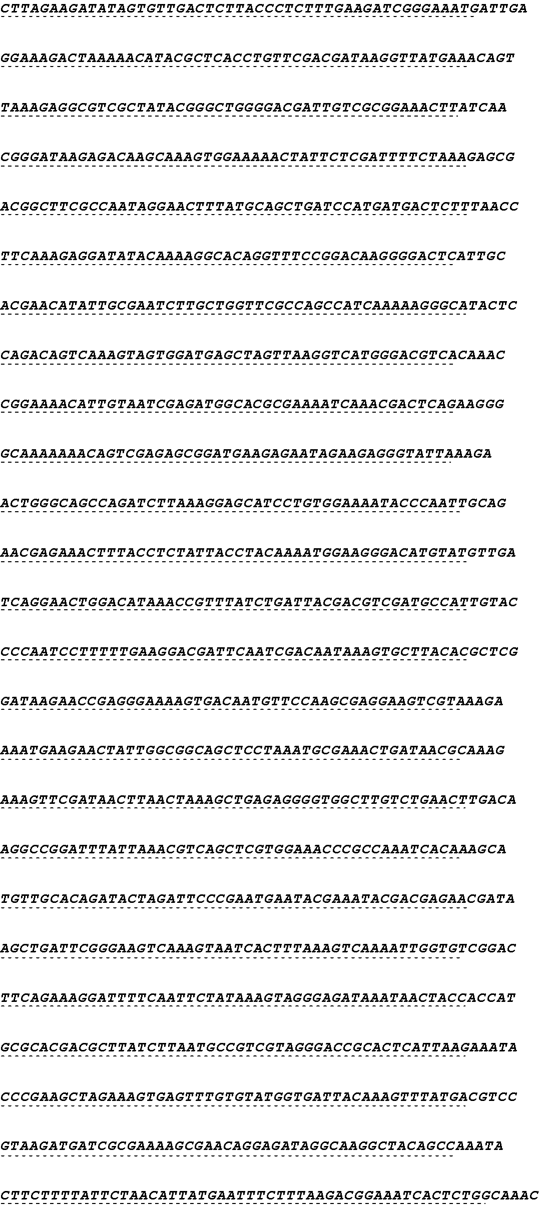

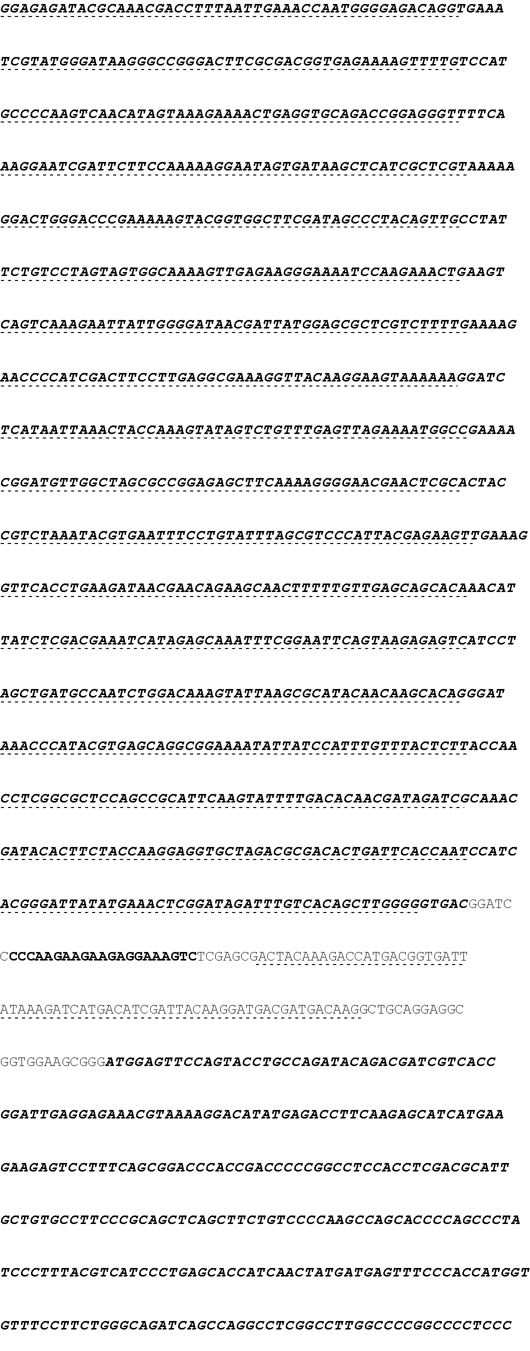

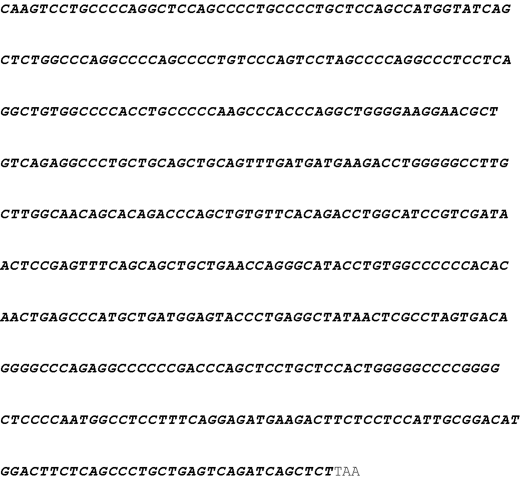

NNNN = NLS

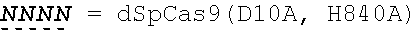

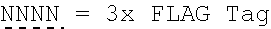

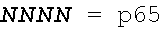

JEH127: pCAG-NLS-dSpCas9(D10A, H840A)-NLS-3xHA-VPR(VP64-p65-RTA)
(SEQ ID NO: 12)
ATGGCGCCGAAAAAAAAACGCAAAGTGAACGGCGGAGGGTCCGGAGGAGGC

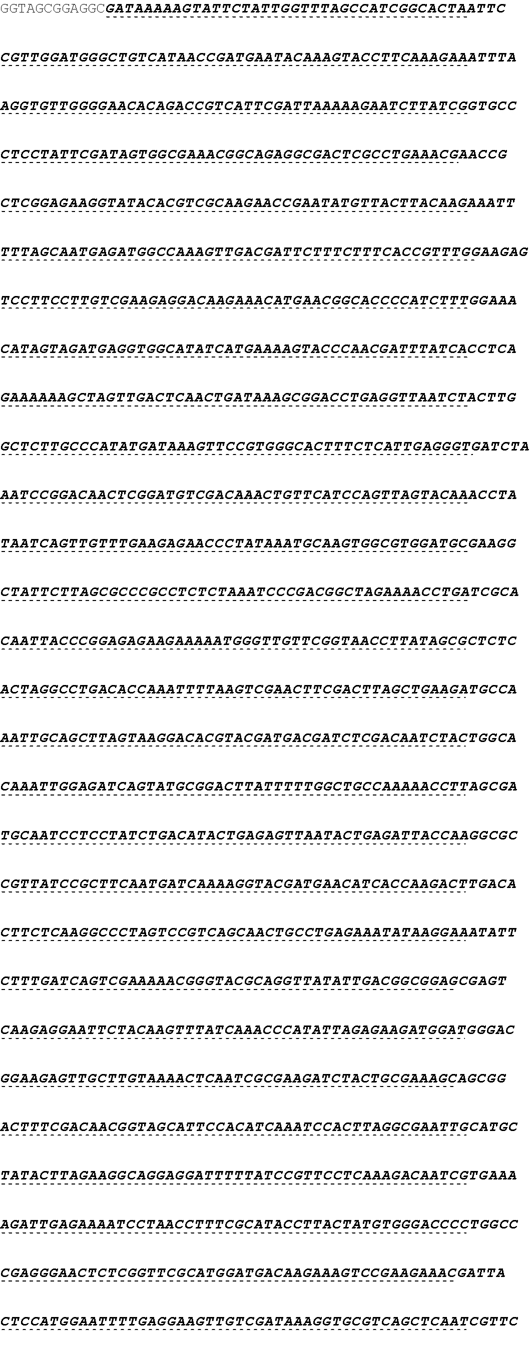

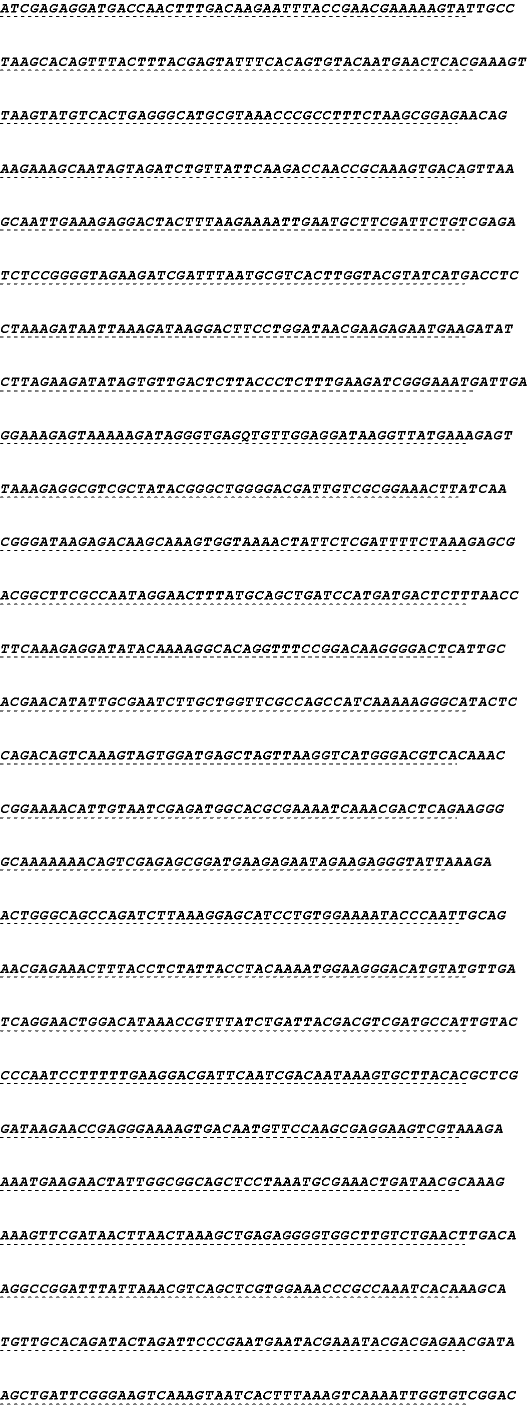

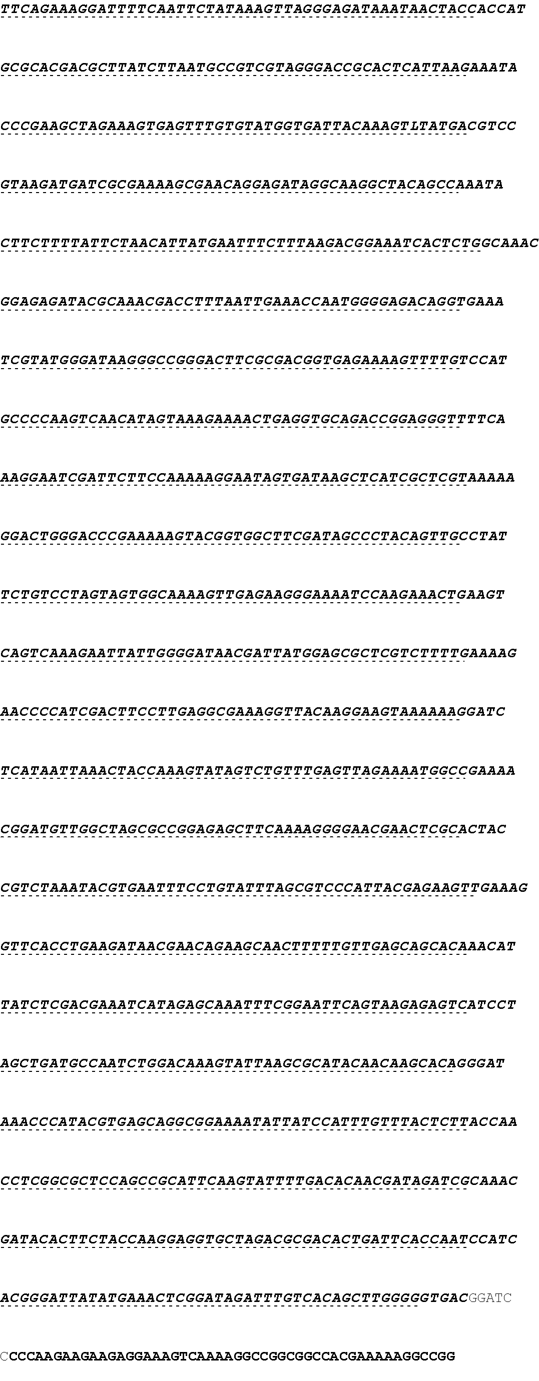

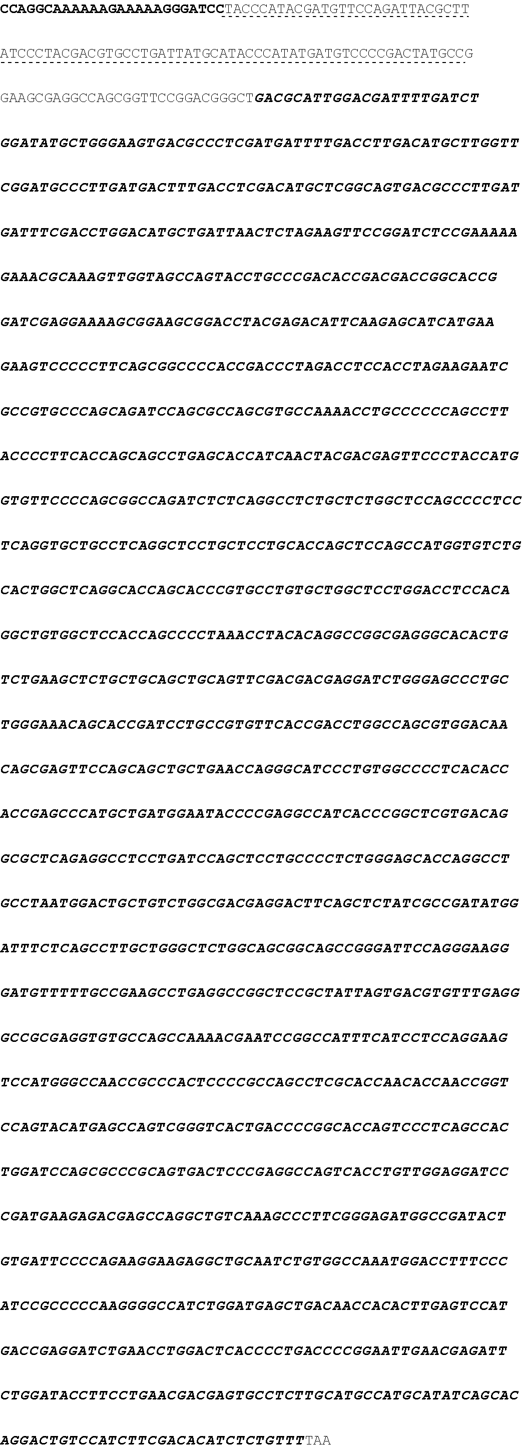

NNNN = NLS

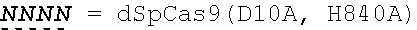

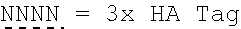

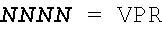

BPK880: pCAG-DmrC-NLS-3xFLAG-VP64
(SEQ ID NO:13)

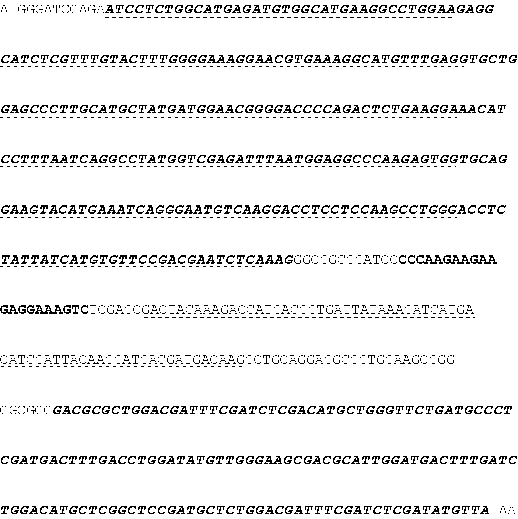

NNNN = NLS

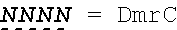

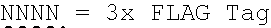

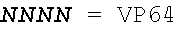

BPK1169: pCAG-DmrC-NLS-3xFLAG-p65
(SEQ ID NO: 14)
GTCGACATTGATTATTGACTAGTTATTAATAGTAATCAATTACGG
GGTCATTAGTTCATAGCCCATATATGGAGTTCCGCGTTACATAAC
TTACGGTAAATGGCCCGCCTGGCTGACCGCCCAACGACCCCCGCC
CATTGACGTCAATAATGACGTATGTTCCCATAGTAACGCCAATAG
GGACTTTCCATTGACGTCAATGGGTGGAGTATTTACGGTAAACTG
CCCACTTGGGAGTAGATCAAGTGTATCATATGCCAAGTACGCCCC
CTATTGACGTCAATGACGGTAAATGGCCCGCCTGGCATTATGCCC
AGTACATGACCTTATGGGACTTTCCTACTTGGCAGTACATCTACG
TATTAGTCATCGCTATTACCATGGTCGAGGTGAGCCCCACGTTCT
GCTTCACTCTCCCCATCTCCCCCCCCTCCCCACCCCCAATTTTGT
ATTTATTTATTTTTTAATTATTTTGTGCAGCGATGGGGGCGGGGG
GGGGGGGGGGCGCGCGCCAGGCGGGGCGGGGCGGGGCGAGGGGCG
GGGCGGGGCGAGGCGGAGAGGTGCGGCGGCAGCCAATCAGAGCGG
CGCGCTCCGAAAGTTTCCTTTTATGGCGAGGCGGCGGCGGCGGCG
GCCCTATAAAAAGCGAAGCGCGCGGCGGGCGGGAGTCGCTGCGCG
CTGCCTTCGCCCCGTGCCCCGCTCCGCCGCCGCCTCGCGCCGCCC
GCCCCGGCTCTGACTGACCGCGTTACTCCCACAGGTGAGCGGGCG
GGACGGCCCTTCTCCTCCGGGCTGTAATTAGCGCTTGGTTTAATG
ACGGCTTGTTTCTTTTCTGTGGCTGCGTGAAAGCCTTGAGGGGCT
CCGGGAGGGCCCTTTGTGCGGGGGGAGCGGCTCGGGGGGTGCGTG
CGTGTGTGTGTGCGTGGGGAGCGCCGCGTGCGGCTCCGCGCTGCC
CGGCGGCTGTGAGCGCTGCGGGCGCGGCGCGGGGCTTTGTGCGCT
CCGCAGTGTGCGCGAGGGGAGCGCGGCCGGGGGCGGTGCCCCGCG
GTGCGGGGGGGGCTGCGAGGGGAACAAAGGCTGCGTGCGGGGTGT
GTGCGTGGGGGGGTGAGCAGGGGGTGTGGGCGCGTCGGTCGGGCT
GCAACCCCCCCTGCACCCCCCTCCCCGAGTTGCTGAGCACGGCCC
GGCTTCGGGTGCGGGGCTCCGTACGGGGCGTGGCGCGGGGCTCGC
CGTGCCGGGCGGGGGGTGGCGGCAGGTGGGGGTGCCGGGCGGGGC
GGGGCCGCCTCGGGCCGGGGAGGGCTCGGGGGAGGGGCGCGGCGG
CCCCCGGAGCGCCGGCGGCTGTCGAGGCGCGGCGAGCCGCAGCCA
TTGCCTTTTATGGTAATCGTGCGAGAGGGCGCAGGGACTTCCTTT
GTCCCAAATCTGTGCGGAGCCGAAATCTGGGAGGCGCCGCCGCAC
CCCCTCTAGCGGGCGCGGGGCGAAGCGGTGCGGCGCCGGCAGGAA
GGAAATGGGCGGGGAGGGCCTTCGTGCGTCGCCGCGCCGCCGTCC
CCTTCTCCCTCTCCAGCCTCGGGGCTGTCCGCGGGGGGACGGCTG
CCTTCGGGGGGGACGGGGCAGGGCGGGGTTCGGCTTCTGGCGTGT
GACCGGCGGCTCTAGAGCCTCTGCTAACCATGTTCATGCCTTCTT
CTTTTTCCTACAGCTCCTGGGCAACGTGCTGGTTATTGTGCTGTC
TCATCATTTTGGCAAAGAATTCTGCAGTCGACGGTACCGCGGGCC
CGGGATCCACCGGTCGCCACCATGGGATCCAGAATCCTCTGGCAT
GAGATGTGGCATGAAGGCCTGGAAGAGGCATCTCGTTTGTACTTT
GGGGAAAGGAACGTGAAAGGCATGTTTGAGGTGCTGGAGCCCTTG
CATGCTATGATGGAACGGGGACCCCAGACTCTGAAGGAAACATCC
TTTAATCAGGCCTATGGTCGAGATTTAATGGAGGCCCAAGAGTGG
TGCAGGAAGTACATGAAATCAGGGAATGTCAAGGACCTCCTCCAA
GCCTGGGACCTCTATTATCATGTGTTCCGACGAATCTCAAAGGGC
GGCGGATCCCCCAAGAAGAAGAGGAAAGTCTCGAGCGACTACAAA
GACCATGACGGTGATTATAAAGATCATGACATCGATTACAAGGAT
GACGATGACAAGGCTGCAGGAGGCGGTGGAAGCGGGATGGAGTTC
CAGTACCTGCCAGATACAGAGGATCGTCACCGGATTGAGGAGAAA
CGTAAAAGGAGATATGAGACCTTCAAGAGCATCATGAAGAAGAGT
CCTTTCAGCGGACCCACCGACCCCCGGCCTCCACCTCGACGCATT
GCTGTGCCTTCCCGCAGCTCAGCTTCTGTCCCCAAGCCAGCACCC
CAGCCCTATCCCTTTACGTCATCCCTGAGCACCATCAACTATGAT
GAGTTTCCCACCATGGTGTTTCCTTCTGGGCAGATCAGCCAGGCC
TCGGCCTTGGCCCCGGCCCCTCCCCAAGTCCTGCCCCAGGCTCCA
GCCCCTGCCCCTGCTCCAGCCATGGTATCAGCTCTGGCCCAGGCC
CCAGCCCCTGTCCCAGTCCTAGCCCCAGGCCCTCCTCAGGCTGTG
GCCCCACCTGCCCCCAAGCCCACCCAGGCTGGGGAAGGAACGCTG
TCAGAGGCCCTGCTGCAGCTGCAGTTTGATGATGAAGACCTGGGG
GCCTTGCTTGGCAACAGCACAGACCCAGCTGTGTTCACAGACCTG
GCATCCGTCGATAACTCCGAGTTTCAGCAGCTGCTGAACCAGGGC
ATACCTGTGGCCCCCCACACAACTGAGCCCATGCTGATGGAGTAC
CCTGAGGCTATAACTCGCCTAGTGACAGGGGCCCAGAGGCCCCCC
GACCCAGCTCCTGCTCCACTGGGGGCCCCGGGGCTCCCCAATGGC
CTCCTTTCAGGAGATGAAGACTTCTCCTCCATTGCGGACATGGAC
TTCTCAGCCCTGCTGAGTCAGATCAGCTCTTAAAGCGGCCGCACT
CCTCAGGTGCAGGCTGCCTATCAGAAGGTGGTGGCTGGTGTGGCC
AATGCCCTGGCTCACAAATACCACTGAGATCTTTTTCCCTCTGCC
AAAAATTATGGGGACATCATGAAGCCCCTTGAGCATCTGACTTCT
GGCTAATAAAGGAAATTTATTTTCATTGCAATAGTGTGTTGGAAT
TTTTTGTGTCTCTCACTCGGAAGGACATATGGGAGGGCAAATCAT
TTAAAACATCAGAATGAGTATTTGGTTTAGAGTTTGGCAACATAT
GCCCATATGCTGGCTGCCATGAACAAAGGTTGGCTATAAAGAGGT
CATCAGTATATGAAACAGCCCCCTGCTGTCCATTCCTTATTCCAT
AGAAAAGCCTTGACTTGAGGTTAGATTTTTTTTATATTTTGTTTT
GTGTTATTTTTTTCTTTAACATCCCTAAAATTTTCCTTACATGTT
TTACTAGCCAGATTTTTCCTCCTCTCCTGACTACTCCCAGTCATA
GCTGTCCCTCTTCTCTTATGGAGATCCCTCGACCTGCAGCCCAAG
CTTGGCGTAATCATGGTCATAGCTGTTTCCTGTGTGAAATTGTTA
TCCGCTCACAATTCCACACAACATACGAGCCGGAAGCATAAAGTG
TAAAGCCTGGGGTGCCTAATGAGTGAGCTAACTCACATTAATTGC
GTTGCGCTCACTGCCCGCTTTCCAGTCGGGAAACCTGTCGTGCCA
GCGGATCCGCATCTCAATTAGTCAGCAACCATAGTCCCGCCCCTA
ACTCCGCCCATCCCGCCCCTAACTCCGCCCAGTTCCGCCCATTCT
CCGCCCCATGGCTGACTAATTTTTTTTATTTATGCAGAGGCCGAG
GCCGCCTCGGCCTCTGAGCTATTCCAGAAGTAGTGAGGAGGCTTT
TTTGGAGGCCTAGGCTTTTGCAAAAAGCTAACTTGTTTATTGCAG
CTTATAATGGTTACAAATAAAGCAATAGCATCACAAATTTCACAA
ATAAAGCATTTTTTTCACTGCATTCTAGTTGTGGTTTGTCCAAAC
TCATCAATGTATCTTATCATGTCTGGATCCGCTGCATTAATGAAT
CGGCCAACGCGCGGGGAGAGGCGGTTTGCGTATTGGGCGCTCTTC
CGCTTCCTCGCTCACTGACTCGCTGCGCTCGGTCGTTCGGCTGCG
GCGAGCGGTATCAGCTCACTCAAAGGCGGTAATACGGTTATCCAC
AGAATCAGGGGATAACGCAGGAAAGAACATGTGAGCAAAAGGCCA
GCAAAAGGCCAGGAACCGTAAAAAGGCCGCGTTGCTGGCGTTTTT
CCATAGGCTCCGCCCCCCTGACGAGCATCACAAAAATCGACGCTC
AAGTCAGAGGTGGCGAAACCCGACAGGACTATAAAGATACCAGGC
GTTTCCCCCTGGAAGCTCCCTCGTGCGCTCTCCTGTTCCGACCCT
GCCGCTTACCGGATACCTGTCCGCCTTTCTCCCTTCGGGAAGCGT
GGCGCTTTCTCATAGCTCACGCTGTAGGTATCTCAGTTCGGTGTA
GGTCGTTCGCTCCAAGCTGGGCTGTGTGCACGAACCCCCCGTTCA
GCCCGACCGCTGCGCCTTATCCGGTAACTATCGTCTTGAGTCCAA
CCCGGTAAGACACGACTTATCGCCACTGGCAGCAGCCACTGGTAA
CAGGATTAGCAGAGCGAGGTATGTAGGCGGTGCTACAGAGTTCTT
GAAGTGGTGGCCTAACTACGGCTACACTAGAAGAACAGTATTTGG
TATCTGCGCTCTGCTGAAGCCAGTTACCTTCGGAAAAAGAGTTGG
TAGCTCTTGATCCGGCAAACAAACCACCGCTGGTAGCGGTGGTTT
TTTTGTTTGCAAGCAGCAGATTACGCGCAGAAAAAAAGGATCTCA
AGAAGATCCTTTGATCTTTTCTACGGGGTCTGACGCTCAGTGGAA
CGAAAACTCACGTTAAGGGATTTTGGTCATGAGATTATCAAAAAG
GATCTTCACCTAGATCCTTTTAAATTAAAAATGAAGTTTTAAATC
AATCTAAAGTATATATGAGTAAACTTGGTCTGACAGTTACCAATG
CTTAATCAGTGAGGCACCTATCTCAGCGATCTGTCTATTTCGTTC
ATCCATAGTTGCCTGACTCCCCGTCGTGTAGATAACTACGATACG
GGAGGGCTTACCATCTGGCCCCAGTGCTGCAATGATACCGCGAGA
CCCACGCTCACCGGCTCCAGATTTATCAGCAATAAACCAGCCAGC
CGGAAGGGCCGAGCGCAGAAGTGGTCCTGCAACTTTATCCGCCTC
CATCCAGTCTATTAATTGTTGCCGGGAAGCTAGAGTAAGTAGTTC
GCCAGTTAATAGTTTGCGCAACGTTGTTGCCATTGCTACAGGCAT
CGTGGTGTCACGCTCGTCGTTTGGTATGGCTTCATTCAGCTCCGG
TTCCCAACGATCAAGGCGAGTTACATGATCCCCCATGTTGTGCAA
AAAAGCGGTTAGCTCCTTCGGTCCTCCGATCGTTGTCAGAAGTAA
GTTGGCCGCAGTGTTATCACTCATGGTTATGGCAGCACTGCATAA
TTCTCTTACTGTCATGCCATCCGTAAGATGCTTTTCTGTGACTGG
TGAGTACTCAACCAAGTCATTCTGAGAATAGTGTATGCGGCGACC
GAGTTGCTCTTGCCCGGCGTCAATACGGGATAATACCGCGCCACA
TAGCAGAACTTTAAAAGTGCTCATCATTGGAAAACGTTCTTCGGG
GCGAAAACTCTCAAGGATCTTACCGCTGTTGAGATCCAGTTCGAT
GTAACCCACTCGTGCACCCAACTGATCTTCAGCATCTTTTACTTT
CACCAGCGTTTCTGGGTGAGCAAAAACAGGAAGGCAAAATGCCGC
AAAAAAGGGAATAAGGGCGACACGGAAATGTTGAATACTCATACT
CTTCCTTTTTCAATATTATTGAAGCATTTATCAGGGTTATTGTCT
CATGAGCGGATACATATTTGAATGTATTTAGAAAAATAAACAAAT
AGGGGTTCCGCGCACATTTCCCCGAAAAGTGCCACCTGG

MMW948: pCAG-DmrC-NLS-3xFLAG-VPR(VP64-p65-RTA)
(SEQ ID NO: 15)
GGTCGAGATTGATTATTGAGTAGTTATTAATAGTAATCAATTACG
GGGTCATTAGTTCATAGCCCATATATGGAGTTCCGCGTTACATAA
CTTACGGTAAATGGCCCGCCTGGCTGACCGCCCAACGACCCCCGC
CCATTGACGTCAATAATGACGTATGTTCCCATAGTAACGCCAATA
GGGACTTTCCATTGACGTCAATGGGTGGAGTATTTACGGTAAACT
GCCGAGTTGGGAGTAGATCAAGTGTATCATATGCCAAGTACGCCC
CCTATTGAGGTCAATGACGGTAAATGGCCCGCCTGGCATTATGCC
CAGTACATGACCTTATGGGACTTTCCTACTTGGCAGTACATCTAC
GTATTAGTCATCGCTATTACCATGGTCGAGGTGAGCCCCACGTTC
TGCTTCACTCTCCCCATCTCCCCCCCCTCCCCACCCCCAATTTTG
TATTTATTTATTTTTTAATTATTTTGTGCAGCGATGGGGGCGGGG
GGGGGGGGGGCGCGCGCCAGGCGGGGCGGGGCGGGGCGAGGGGSG
GGGCGGGGCGAGGCGGAGAGGTGCGGCGGCAGCCAATCAGAGCGG
CGCGCTCCGAAAGTTTCCTTTTATGGCGAGGCGGCGGCGGCGGCG
GCCCTATAAAAAGCGAAGCGCGCGGCGGGCGGGAGTCGCTGCGCG
CTGCCTTCGCCCCGTGCCCCGCTCCGCCGCCGCCTCGCGCCGCCC
GCCCCGGCTCTGACTGACCGCGTTACTCCCACAGGTGAGCGGGCG
GGACGGCCCTTCTCCTCCGGGCTGTAATTAGCGCTTGGTTTAATG
ACGGCTTGTTTCTTTTCTGTGGCTGCGTGAAAGCCTTGAGGGGCT
CCGGGAGGGCCCTTTGTGCGGGGGGAGCGGCTCGGGGGGTGCGTG
CGTGTGTGTGTGCGTGGGGAGCGCCGCGTGCGGCTCCGCGCTGCC
CGGCGGCTGTGAGCGCTGCGGGCGCGGCGCGGGGCTTTGTGCGCT
CCGCAGTGTGCGCGAGGGGAGCGCGGCCGGGGGCGGTGCCCCGCG
GTGCGGGGGGGGCTGCGAGGGGAACAAAGGCTGCGTGCGGGGTGT
GTGCGTGGGGGGGTGAGCAGGGGGTGTGGGCGCGTCGGTCGGGCT
GCAACCCCCCCTGCACCCCCCTCCCCGAGTTGCTGAGCACGGCCC
GGCTTCGGGTGCGGGGCTCCGTACGGGGCGTGGCGCGGGGCTCGC
CGTGCCGGGCGGGGGGTGGCGGCAGGTGGGGGTGCCGGGCGGGGC
GGGGCCGCCTCGGGCCGGGGAGGGCTCGGGGGAGGGGCGCGGCGG
CCCCCGGAGCGCCGGCGGCTGTCGAGGCGCGGCGAGCCGCAGCCA
TTGCCTTTTATGGTAATCGTGCGAGAGGGCGCAGGGACTTCCTTT
GTCCCAAATCTGTGCGGAGCCGAAATCTGGGAGGCGCCGCCGCAC
CCCCTCTAGCGGGCGCGGGGCGAAGCGGTGCGGCGCCGGCAGGAA
GGAAATGGGCGGGGAGGGCCTTCGTGCGTCGCCGCGCCGCCGTCC
CCTTCTCCCTCTCCAGCCTCGGGGCTGTCCGCGGGGGGACGGCTG
CCTTCGGGGGGGACGGGGCAGGGCGGGGTTCGGCTTCTGGCGTGT
GACCGGCGGCTCTAGAGCCTCTGCTAACCATGTTCATGCCTTCTT
CTTTTTCCTACAGCTCCTGGGCAACGTGCTGGTTATTGTGCTGTC
TCATCATTTTGGCAAAGAATTCTGCAGTCGACGGTACCGCGGGCC
CGGGATCCACCGGTCGCCACCATGGGATCCAGAATCCTCTGGCAT
GAGATGTGGCATGAAGGCCTGGAAGAGGCATCTCGTTTGTACTTT
GGGGAAAGGAACGTGAAAGGCATGTTTGAGGTGCTGGAGCCCTTG
CATGCTATGATGGAACGGGGACCCCAGACTCTGAAGGAAACATCC
TTTAATCAGGCCTATGGTCGAGATTTAATGGAGGCCCAAGAGTGG
TGCAGGAAGTACATGAAATCAGGGAATGTCAAGGACCTCCTCCAA
GCCTGGGACCTCTATTATCATGTGTTCCGACGAATCTCAAAGGGC
GGCGGATCCCCCAAGAAGAAGAGGAAAGTCTCGAGCGACTACAAA
GACCATGACGGTGATTATAAAGATCATGACATCGATTACAAGGAT
GACGATGACAAGGCTGCAGGAGGCGGTGGAAGCGGGTCGGAGGCC
AGCGGTTCCGGACGGGCTGACGCATTGGACGATTTTGATCTGGAT
ATGCTGGGAAGTGACGCCCTCGATGATTTTGACCTTGACATGCTT
GGTTCGGATGCCCTTGATGACTTTGACCTCGACATGCTCGGCAGT
GACGCCCTTGATGATTTCGACCTGGACATGCTGATTAACTCTAGA
AGTTCCGGATCTCCGAAAAAGAAACGCAAAGTTGGTAGCCAGTAC
CTGCCCGACACCGACGACCGGCACCGGATCGAGGAAAAGCGGAAG
CGGACCTACGAGACATTCAAGAGCATCATGAAGAAGTCCCCCTTC
AGCGGCCCCACCGACCCTAGACCTCCACCTAGAAGAATCGCCGTG
CCCAGCAGATCCAGCGCCAGCGTGCCAAAACCTGCCCCCCAGCCT
TACCCCTTCACCAGCAGCCTGAGCACCATCAACTACGACGAGTTC
CCTACCATGGTGTTCCCCAGCGGCCAGATCTCTCAGGCCTCTGCT
CTGGCTCCAGCCCCTCCTCAGGTGCTGCCTCAGGCTCCTGCTCCT
GCACCAGCTCCAGCCATGGTGTCTGCACTGGCTCAGGCACCAGCA
CCCGTGCCTGTGCTGGCTCCTGGACCTCCACAGGCTGTGGCTCCA
CCAGCCCCTAAACCTACACAGGCCGGCGAGGGCACACTGTCTGAA
GCTCTGCTGCAGCTGCAGTTCGACGACGAGGATCTGGGAGCCCTG
CTGGGAAACAGCACCGATCCTGCCGTGTTCACCGACCTGGCCAGC
GTGGACAACAGCGAGTTCCAGCAGCTGCTGAACCAGGGCATCCCT
GTGGCCCCTCACACCACCGAGCCCATGCTGATGGAATACCCCGAG
GCCATCACCCGGCTCGTGACAGGCGCTCAGAGGCCTCCTGATCCA
GCTCCTGCCCCTCTGGGAGCACCAGGCCTGCCTAATGGACTGCTG
TCTGGCGACGAGGACTTCAGCTCTATCGCCGATATGGATTTCTCA
GCCTTGCTGGGCTCTGGCAGCGGCAGCCGGGATTCCAGGGAAGGG
ATGTTTTTGCCGAAGCCTGAGGCCGGCTCCGCTATTAGTGACGTG
TTTGAGGGCCGCGAGGTGTGCCAGCCAAAACGAATCCGGCCATTT
CATCCTCCAGGAAGTCCATGGGCCAACCGCCCACTCCCCGCCAGC
CTCGCACCAACACCAACCGGTCCAGTACATGAGCCAGTCGGGTCA
CTGACCCCGGCACCAGTCCCTCAGCCACTGGATCCAGCGCCCGCA
GTGACTCCCGAGGCCAGTCACCTGTTGGAGGATCCCGATGAAGAG
ACGAGCCAGGCTGTCAAAGCCCTTCGGGAGATGGCCGATACTGTG
ATTCCCCAGAAGGAAGAGGCTGCAATCTGTGGCCAAATGGACCTT
TCCCATCCGCCCCCAAGGGGCCATCTGGATGAGCTGACAACCACA
CTTGAGTCCATGACCGAGGATCTGAACCTGGACTCACCCCTGACC
CCGGAATTGAACGAGATTCTGGATACCTTCCTGAACGACGAGTGC
CTCTTGCATGCCATGCATATCAGCACAGGACTGTCCATCTTCGAC
ACATCTCTGTTTTAAAGCGGCCGCACTCCTCAGGTGCAGGCTGCC
TATCAGAAGGTGGTGGCTGGTGTGGCCAATGCCCTGGCTCACAAA
TACCACTGAGATCTTTTTCCCTCTGCCAAAAATTATGGGGACATC
ATGAAGCCCCTTGAGCATCTGACTTCTGGCTAATAAAGGAAATTT
ATTTTCATTGCAATAGTGTGTTGGAATTTTTTGTGTCTCTCACTC
GGAAGGACATATGGGAGGGCAAATCATTTAAAACATCAGAATGAG
TATTTGGTTTAGAGTTTGGCAACATATGCCCATATGCTGGCTGCC
ATGAACAAAGGTTGGCTATAAAGAGGTCATCAGTATATGAAACAG
CCCCCTGCTGTCCATTCCTTATTCCATAGAAAAGCCTTGACTTGA
GGTTAGATTTTTTTTATATTTTGTTTTGTGTTATTTTTTTCTTTA
ACATCCCTAAAATTTTCCTTACATGTTTTACTAGCCAGATTTTTC
CTCCTCTCCTGACTACTCCCAGTCATAGCTGTCCCTCTTCTCTTA
TGGAGATCCCTCGACCTGCAGCCCAAGCTTGGCGTAATCATGGTC
ATAGCTGTTTCCTGTGTGAAATTGTTATCCGCTCACAATTCCACA
CAACATACGAGCCGGAAGCATAAAGTGTAAAGCCTGGGGTGCCTA
ATGAGTGAGCTAACTCACATTAATTGCGTTGCGCTCACTGCCCGC
TTTCCAGTCGGGAAACCTGTCGTGCCAGCGGATCCGCATCTCAAT
TAGTCAGCAACCATAGTCCCGCCCCTAACTCCGCCCATCCCGCCC
CTAACTCCGCCCAGTTCCGCCCATTCTCCGCCCCATGGCTGACTA
ATTTTTTTTATTTATGCAGAGGCCGAGGCCGCCTCGGCCTCTGAG
CTATTCCAGAAGTAGTGAGGAGGCTTTTTTGGAGGCCTAGGCTTT
TGCAAAAAGCTAACTTGTTTATTGCAGCTTATAATGGTTACAAAT
AAAGCAATAGCATCACAAATTTCACAAATAAAGCATTTTTTTCAC
TGCATTCTAGTTGTGGTTTGTCCAAACTCATCAATGTATCTTATC
ATGTCTGGATCCGCTGCATTAATGAATCGGCCAACGCGCGGGGAG
AGGCGGTTTGCGTATTGGGCGCTCTTCCGCTTCCTCGCTCACTGA
CTCGCTGCGCTCGGTCGTTCGGCTGCGGCGAGCGGTATCAGCTCA
CTCAAAGGCGGTAATACGGTTATCCACAGAATCAGGGGATAACGC
AGGAAAGAACATGTGAGCAAAAGGCCAGCAAAAGGCCAGGAACCG
TAAAAAGGCCGCGTTGCTGGCGTTTTTCCATAGGCTCCGCCCCCC
TGACGAGCATCACAAAAATCGACGCTCAAGTCAGAGGTGGCGAAA
CCCGACAGGACTATAAAGATACCAGGCGTTTCCCCCTGGAAGCTC
CCTCGTGCGCTCTCCTGTTCCGACCCTGCCGCTTACCGGATACCT
GTCCGCCTTTCTCCCTTCGGGAAGCGTGGCGCTTTCTCATAGCTC
ACGCTGTAGGTATCTCAGTTCGGTGTAGGTCGTTCGCTCCAAGCT
GGGCTGTGTGCACGAACCCCCCGTTCAGCCCGACCGCTGCGCCTT
ATCCGGTAACTATCGTCTTGAGTCCAACCCGGTAAGACACGACTT
ATCGCCACTGGCAGCAGCCACTGGTAACAGGATTAGCAGAGCGAG
GTATGTAGGCGGTGCTACAGAGTTCTTGAAGTGGTGGCCTAACTA
CGGCTACACTAGAAGAACAGTATTTGGTATCTGCGCTCTGCTGAA
GCCAGTTACCTTCGGAAAAAGAGTTGGTAGCTCTTGATCCGGCAA
ACAAACCACCGCTGGTAGCGGTGGTTTTTTTGTTTGCAAGCAGCA
GATTACGCGCAGAAAAAAAGGATCTCAAGAAGATCCTTTGATCTT
TTCTACGGGGTCTGACGCTCAGTGGAACGAAAACTCACGTTAAGG
GATTTTGGTCATGAGATTATCAAAAAGGATCTTCACCTAGATCCT
TTTAAATTAAAAATGAAGTTTTAAATCAATCTAAAGTATATATGA
GTAAACTTGGTCTGACAGTTACCAATGCTTAATCAGTGAGGCACC
TATCTCAGCGATCTGTCTATTTCGTTCATCCATAGTTGCCTGACT
CCCCGTCGTGTAGATAACTACGATACGGGAGGGCTTACCATCTGG
CCCCAGTGCTGCAATGATACCGCGAGACCCACGCTCACCGGCTCC
AGATTTATCAGCAATAAACCAGCCAGCCGGAAGGGCCGAGCGCAG
AAGTGGTCCTGCAACTTTATCCGCCTCCATCCAGTCTATTAATTG
TTGCCGGGAAGCTAGAGTAAGTAGTTCGCCAGTTAATAGTTTGCG
CAACGTTGTTGCCATTGCTACAGGCATCGTGGTGTCACGCTCGTC
GTTTGGTATGGCTTCATTCAGCTCCGGTTCCCAACGATCAAGGCG
AGTTACATGATCCCCCATGTTGTGCAAAAAAGCGGTTAGCTCCTT
CGGTCCTCCGATCGTTGTCAGAAGTAAGTTGGCCGCAGTGTTATC
ACTCATGGTTATGGCAGCACTGCATAATTCTCTTACTGTCATGCC
ATCCGTAAGATGCTTTTCTGTGACTGGTGAGTACTCAACCAAGTC
ATTCTGAGAATAGTGTATGCGGCGACCGAGTTGCTCTTGCCCGGC
GTCAATACGGGATAATACCGCGCCACATAGCAGAACTTTAAAAGT
GCTCATCATTGGAAAACGTTCTTCGGGGCGAAAACTCTCAAGGAT
CTTACCGCTGTTGAGATCCAGTTCGATGTAACCCACTCGTGCACC
CAACTGATCTTCAGCATCTTTTACTTTCACCAGCGTTTCTGGGTG
AGCAAAAACAGGAAGGCAAAATGCCGCAAAAAAGGGAATAAGGGC
GACACGGAAATGTTGAATACTCATACTCTTCCTTTTTCAATATTA
TTGAAGCATTTATCAGGGTTATTGTCTCATGAGCGGATACATATT
TGAATGTATTTAGAAAAATAAACAAATAGGGGTTCCGCGCACATT
TCCCCGAAAAGTGCCACCTG

BPK1520 (pU6-BsmBICassette-S.pyogenes.sgRNA)
(SEQ ID NO: 16)
CGAGGTACCTCTCTACATATGACATGTGAGCAAAAGGCCAGCAAA
AGGCCAGGAACCGTAAAAAGGCCGCGTTGCTGGCGTTTTTCCATA
GGCTCCGCCCCCCTGACGAGCATCACAAAAATCGACGCTCAAGTC
AGAGGTGGCGAAACCCGACAGGACTATAAAGATACCAGGCGTTTC
CCCCTGGAAGCTCCCTCGTGCGCTCTCCTGTTCCGACCCTGCCGC
TTACCGGATACCTGTCCGCCTTTCTCCCTTCGGGAAGCGTGGCGC
TTTCTCATAGCTCACGCTGTAGGTATCTCAGTTCGGTGTAGGTCG
TTCGCTCCAAGCTGGGCTGTGTGCACGAACCCCCCGTTCAGCCCG
ACCGCTGCGCCTTATCCGGTAACTATCGTCTTGAGTCCAACCCGG
TAAGACACGACTTATCGCCACTGGCAGCAGCCACTGGTAACAGGA
TTAGCAGAGCGAGGTATGTAGGCGGTGCTACAGAGTTCTTGAAGT
GGTGGCCTAACTACGGCTACACTAGAAGAACAGTATTTGGTATCT
GCGCTCTGCTGAAGCCAGTTACCTTCGGAAAAAGAGTTGGTAGCT
CTTGATCCGGCAAACAAACCACCGCTGGTAGCGGTGGTTTTTTTG
TTTGCAAGCAGCAGATTACGCGCAGAAAAAAAGGATCTCAAGAAG
ATCCTTTGATCTTTTCTACGGGGTCTGACGCTCAGTGGAACGAAA
ACTCACGTTAAGGGATTTTGGTCATGAGATTATCAAAAAGGATCT
TCACCTAGATCCTTTTAAATTAAAAATGAAGTTTTAAATCAATCT
AAAGTATATATGAGTAAACTTGGTCTGACAGTTACCAATGCTTAA
TCAGTGAGGCACCTATCTCAGCGATCTGTCTATTTCGTTCATCCA
TAGTTGCCTGACTCCCCGTCGTGTAGATAACTACGATACGGGAGG
GCTTACCATCTGGCCCCAGTGCTGCAATGATACCGCGAGACCCAC
GCTCACCGGCTCCAGATTTATCAGCAATAAACCAGCCAGCCGGAA
GGGCCGAGCGCAGAAGTGGTCCTGCAACTTTATCCGCCTCCATCC
AGTCTATTAATTGTTGCCGGGAAGCTAGAGTAAGTAGTTCGCCAG
TTAATAGTTTGCGCAACGTTGTTGCCATTGCTACAGGCATCGTGG
TGTCACGCTCGTCGTTTGGTATGGCTTCATTCAGCTCCGGTTCCC
AACGATCAAGGCGAGTTACATGATCCCCCATGTTGTGCAAAAAAG
CGGTTAGCTCCTTCGGTCCTCCGATCGTTGTCAGAAGTAAGTTGG
CCGCAGTGTTATCACTCATGGTTATGGCAGCACTGCATAATTCTC
TTACTGTCATGCCATCCGTAAGATGCTTTTCTGTGACTGGTGAGT
ACTCAACCAAGTCATTCTGAGAATAGTGTATGCGGCGACCGAGTT
GCTCTTGCCCGGCGTCAATACGGGATAATACCGCGCCACATAGCA
GAACTTTAAAAGTGCTCATCATTGGAAAACGTTCTTCGGGGCGAA
AACTCTCAAGGATCTTACCGCTGTTGAGATCCAGTTCGATGTAAC
CCACTCGTGCACCCAACTGATCTTCAGCATCTTTTAGTTTCACCA
GCGTTTCTGGGTGAGCAAAAACAGGAAGGCAAAATGCCGCAAAAA
AGGGAATAAGGGCGACACGGAAATGTTGAATACTCATACTCTTCC
TTTTTCAATATTATTGAAGCATTTATCAGGGTTATTGTCTCATGA
GCGGATACATATTTGAATGTATTTAGAAAAATAAACAAATAGGGG
TTCCGCGCACATTTCCCCGAAAAGTGCCACCTGACGTCGCTAGCT
GTACAAAAAAGCAGGCTTTAAAGGAACCAATTCAGTCGACTGGAT
CCGGTACCAAGGTCGGGCAGGAAGAGGGCCTATTTCCCATGATTC
CTTCATATTTGCATATACGATACAAGGCTGTTAGAGAGATAATTA
GAATTAATTTGACTGTAAACACAAAGATATTAGTACAAAATACGT
GACGTAGAAAGTAATAATTTCTTGGGTAGTTTGCAGTTTTAAAAT
TATGTTTTAAAATGGACTATCATATGCTTACCGTAACTTGAAAGT
ATTTCGATTTCTTGGCTTTATATATCTTGTGGAAAGGACGAAACA
CCGGAGACGATTAATGCGTCTCCGTTTTAGAGCTAGAAATAGCAA
GTTAAAATAAGGCTAGTCCGTTATCAACTTGAAAAAGTGGCACCG
AGTCGGTGCTTTTTTTAAGCTTGGGCCGCT

TABLE 7
Cas9 gRNAs used in this study

| | | Anno- | | | | | | | |
|---|---|---|---|---|---|---|---|---|---|
| | | ta- | | | | | | | |
| | | tion | | Cas | | | | | |
| Target | Guide | in | | ortho- | | | | | |
| region | name | Figs | Spacer | log | Chr | start | stop | Strand | # |
| IL2RA | YET890 | P1 | CTAGT | SpCas9 | chr10 | 6104772 | 6104791 | − | 17 |
| promoter | | | ACAGG | | | | | | |
| | | | TTATA | | | | | | |
| | | | AGCCT | | | | | | |
| | YET891 | P2 | TCTTC | SpCas9 | chr10 | 6104505 | 6104524 | + | 18 |
| | | | AGCTA | | | | | | |
| | | | CGCCC | | | | | | |
| | | | ATAAA | | | | | | |
| | YET728 | P3 | TTATG | SpCas9 | chr10 | 6104504 | 6104523 | − | 19 |
| | | | GGCGT | | | | | | |
| | | | AGCTG | | | | | | |
| | | | AAGAA | | | | | | |
| | YET731 | P4 | ATAAG | SpCas9 | chr10 | 6104600 | 6104619 | + | 20 |
| | | | CTGAG | | | | | | |
| | | | TCCTC | | | | | | |
| | | | CCCTC | | | | | | |
| IL2RA | YET718 | E1 | TCTGA | SpCas9 | chr10 | 6094683 | 6094702 | + | 21 |
| enhancers | | | AGGAG | | | | | | |
| | | | GTATC | | | | | | |
| | | | TATTT | | | | | | |
| | YET719 | E2 | GTGGG | SpCas9 | chr10 | 6094722 | 6094741 | + | 22 |
| | | | TGATT | | | | | | |
| | | | CTGTG | | | | | | |
| | | | GGCAG | | | | | | |
| | YET722 | E3 | ACTGT | SpCas9 | chr10 | 6110448 | 6110467 | − | 23 |
| | | | GAGCG | | | | | | |
| | | | TCCTC | | | | | | |
| | | | AGTGC | | | | | | |
| | YET725 | E4 | CCACG | SpCas9 | chr10 | 6110677 | 6110696 | − | 24 |
| | | | CTCCC | | | | | | |
| | | | AGAAA | | | | | | |
| | | | GCAAA | | | | | | |
| CD69 | YET892 | P1 | TCACA | SpCas9 | chr12 | 9913679 | 9913698 | + | 25 |
| promoter | | | ACTGT | | | | | | |
| | | | AAGGT | | | | | | |
| | | | GTAGC | | | | | | |
| | YET899 | P2 | TATCT | SpCas9 | chr12 | 9913192 | 9913211 | − | 26 |
| | | | AATGG | | | | | | |
| | | | TTGTT | | | | | | |
| | | | AGGGA | | | | | | |
| | YET511 | P3 | CAATG | SpCas9 | chr12 | 9913617 | 9913636 | + | 27 |
| | | | TATAG | | | | | | |
| | | | TGTGT | | | | | | |
| | | | TGTTG | | | | | | |
| | YET515 | P4 | TCAAG | SpCas9 | chr12 | 9913557 | 9913576 | − | 28 |
| | | | CAAGT | | | | | | |
| | | | AGGCG | | | | | | |
| | | | GCAAG | | | | | | |
| CD69 | YET509 | E1 | CTGAG | SpCas9 | chr12 | 9917335 | 9917354 | + | 29 |
| enhancer | | | AAAAT | | | | | | |
| (CNS) | | | AGTAT | | | | | | |
| | | | AGCCG | | | | | | |
| | YET511 | E2 | TCCTT | SpCas9 | chr12 | 9917458 | 9917477 | − | 30 |
| | | | TCTGA | | | | | | |
| | | | CGTCT | | | | | | |
| | | | CACCC | | | | | | |
| MyoD1 | NP85 | P1 | GCCTG | SpCas9 | chr11 | 17741055 | 17741074 | + | 31 |
| promoter | | | GGCTC | | | | | | |
| | | | CGGGG | | | | | | |
| | | | CGTTT | | | | | | |
| | NP83 | P2 | GGGCC | SpCas9 | chr11 | 17740968 | 17740987 | + | 32 |
| | | | CCTGC | | | | | | |
| | | | GGCCA | | | | | | |
| | | | CCCCG | | | | | | |
| | NP86 | P3 | CCTCC | SpCas9 | chr11 | 17740896 | 17740915 | + | 33 |
| | | | CTCCC | | | | | | |
| | | | TGCCC | | | | | | |
| | | | GGTAG | | | | | | |
| | NP84 | P4 | GAGGT | SpCas9 | chr11 | 17740836 | 17740855 | + | 34 |
| | | | TTGGA | | | | | | |
| | | | AAGGG | | | | | | |
| | | | CGTGC | | | | | | |
| MyoD1 | NP79 | E1 | CCAAC | SpCas9 | chr11 | 17721346 | 17721365 | + | 35 |
| enhancer | | | TGAGT | | | | | | |
| | | | CCTGA | | | | | | |
| | | | GGTTT | | | | | | |
| | NP80 | E2 | ACTCA | SpCas9 | chr11 | 17721256 | 17721275 | + | 3 |
| | | | CAGCA | | | | | | |
| | | | CAGCC | | | | | | |
| | | | AGTGT | | | | | | |
| | NP81 | E3 | CCAGC | SpCas9 | chr11 | 17721199 | 17721218 | + | 37 |
| | | | AGCTG | | | | | | |
| | | | GTCAC | | | | | | |
| | | | AAAGC | | | | | | |
| | NP82 | E4 | CCTTC | SpCas9 | chr11 | 17721138 | 17721157 | + | 39 |
| | | | CTATA | | | | | | |
| | | | AACTT | | | | | | |
| | | | CTG | | | | | | |
| | | | AG | | | | | | |
| HBE | YET420 | P_{E} | CTTGG | SpCas9 | chr11 | 5291774 | 5291793 | + | 39 |
| promoter | | | GGTGA | | | | | | |
| | | | TTCCC | | | | | | |
| | | | TAGAG | | | | | | |
| HBG1 | YET192 | P_{G1/2} | GCTAC | SpCas9 | chr11 | 5270988 | 5271007 | − | 40 |
| promoter | | | TATCA | | | | | | |
| | | | CAAGC | | | | | | |
| | | | CTGTG | | | | | | |
| HBG2 | YET192 | P_{G1/2} | GCTAC | SpCas9 | chr11 | 5275912 | 5275931 | − | 41 |
| promoter | | | TATCA | | | | | | |
| | | | CAAGC | | | | | | |
| | | | CTGTG | | | | | | |
| HBB | YET429 | P_{B} | GGCTC | SpCas9 | chr11 | 5248439 | 5248458 | + | 42 |
| promoter | | | TTCTG | | | | | | |
| | | | GCACT | | | | | | |
| | | | GGCTT | | | | | | |
| HS2 | JG891 | E0 | GAAGG | SpCas9 | chr11 | 5302033 | 5302052 | + | 43 |
| enhancer | | | TTACA | | | | | | |
| | | | CAGAA | | | | | | |
| | | | CCAGA | | | | | | |
| APOC3 | JEH612 | P | TGACC | SpCas9 | chr11 | 116700542 | 116700561 | − | 44 |
| promoter | | | TTTGC | | | | | | |
| | | | CCAGC | | | | | | |
| | | | GCCCT | | | | | | |
| APOC3 | JEH599 | E0 | CTGCG | SpCas9 | chr11 | 116699673 | 116699692 | − | 45 |
| enhancer | | | GGGAG | | | | | | |
| | | | TCGGT | | | | | | |
| | | | GGTCC | | | | | | |
| | NP190 | E1 | GCATC | SpCas9 | chr11 | 116696682 | 116696701 | − | 46 |
| | | | TGACC | | | | | | |
| | | | CCAAC | | | | | | |
| | | | AATCA | | | | | | |
| | NP251 | E2 | TGCAA | SpCas9 | chr11 | 116696865 | 116696884 | + | 47 |
| | | | CAATG | | | | | | |
| | | | TCTGG | | | | | | |
| | | | CACAT | | | | | | |
| | NP252 | E3 | CCAGG | SpCas9 | chr11 | 116696924 | 116696943 | − | 48 |
| | | | GAGGT | | | | | | |
| | | | GAGCC | | | | | | |
| | | | TTGCA | | | | | | |
| | NP193 | E4 | CTCTC | SpCas9 | chr11 | 116697119 | 116697138 | − | 49 |
| | | | ACATG | | | | | | |
| | | | CTAGT | | | | | | |
| | | | GGCAC | | | | | | |
| | NP196 | E5 | TCCCC | SpCas9 | chr11 | 116697324 | 116697343 | − | 50 |
| | | | GCCAC | | | | | | |
| | | | GTTGA | | | | | | |
| | | | AAGGC | | | | | | |
| | NP205 | E6 | CAGGC | SpCas9 | chr11 | 116697541 | 116697560 | − | 51 |
| | | | CTTTC | | | | | | |
| | | | ATGCC | | | | | | |
| | | | CACTA | | | | | | |
| Non- | YET519 | None | GGCTC | SpCas9 | N/A | N/A | N/A | N/A | 52 |
| Targeting | | | GGTCC | | | | | | |
| Guide | | | CGCGT | | | | | | |
| | | | CGTCG | | | | | | |

TABLE 8
RT-qPCR primers used in this study

| | Forward | # | Reverse | # |
|---|---|---|---|---|
| IL2RA | GAGACTTCCT | 53 | GATCAGCAGG | 60 |
| | GCCTCGTCAC | | AAAACACAGC | |
| | AA | | CG | |
| CD69 | GCTGGACTTC | 54 | AGTCCAACCC | 61 |
| | AGCCCAAAAT | | AGTGTTCCTC | |
| | GC | | TC | |
| MyoD1 | CTCCAACTGC | 55 | ACAGGCAGTC | 62 |
| | TCCGACGGCA | | TAGGCTCGAC | |
| | T | | AC | |
| HBE | TCACTAGCAA | 56 | AACAACGAGG | 63 |
| | GCTCTCAGGC | | AGTCTGCCC | |
| HBG1/2 | GCTGAGTGAA | 57 | GAATTCTTTG | 64 |
| | CTGCACTGTG | | CCGAAATGGA | |
| | A | | | |
| HBB | GCACGTGGAT | 58 | ATTGGACAGC | 65 |
| | CCTGAGAACT | | AAGAAAGCGA | |
| | | | G | |
| APOC3 | CTCTGCCCGA | 59 | TGTCCTTAAC | 66 |
| | GCTTCAGAG | | GGTGCTCCAG | |

#SEQ ID NO

TABLE 9
Allele-specific RT-qPCR primers
used in this study

| | Forward | # | Reverse | # | Note |
|---|---|---|---|---|---|
| APOC | CTCCTTG | 67 | GGTCTTG | 69 | T- |
| (T/C) | TTGTTGC | | GTGGCGT | | specific |
| 3 exon | CCTCCT | | GCTTCAT | | |
| | | | GTTA | | |
| | CTCCTTG | 68 | GGTCTTG | 70 | C- |
| | TTGTTGC | | GTGGCGT | | specific |
| | CCTCCT | | GCTTCAT | | |
| | | | GT | | |
| | | | TG | | |

#SEQ ID NO

TABLE 10
ChlP-qPCR primers used in this study

| | | SEQ | | SEQ | Product | |
|---|---|---|---|---|---|---|
| Target | | ID | | ID | size | Genomic |
| region | Forward | NO: | Reverse | NO: | (bp) | coordinates |
| E1 | ATGCTGCCTCCTT | 71 | GTACGTGTTGG | 81 | 103 | chr11:1166966 |
| | TTTGATG | | AGCCTGGAT | | | 42-116696744 |
| E2 | GTGCTTGCAACAA | 72 | GGGGCACTGAG | 82 | 164 | chr11:1166968 |
| | TGTCTGG | | TACTGACCT | | | 60-116697023 |
| E3 | GTGCTTGCAACAA | 73 | GGGGCACTGAG | 83 | 164 | chr11:1166968 |
| | TGTCTGG | | TACTGACCT | | | 60-116697023 |
| E4 | GGTGCCACTAGCA | 74 | CACACACTCTG | 84 | 121 | chr11:1166971 |
| | TGTGAGA | | GTGGATGCT | | | 18-116697238 |
| E5 | ATTTGAGCATGTC | 75 | TGATGCAAAAC | 85 | 199 | chr11:1166972 |
| | CGAGAGC | | GTACCCTCA | | | 02-116697400 |
| E6 | AGGGTGCAAGTA | 76 | TGCCTGTTGCAC | 86 | 153 | chr11:1166974 |
| | GCTGATGG | | AGATAAGG | | | 65-116697617 |
| E₀ | ATCTCAGCCCCGA | 77 | CAACTGGGAGG | 87 | 100 | chr11:1166996 |
| | GAAGG | | AACAAGGTC | | | 39-116699738 |
| APOC3 | ATCTCCACTGGTC | 78 | CAGCTGCCTCT | 88 | 128 | chr11:1167005 |
| Promoter | AGCAGGT | | AGGGATGAA | | | 23-116700650 |
| Non- | AACCCTGCTTATC | 79 | CTCTGCCCTGCC | 89 | 73 | chr11:5255727 |
| Target | TTAAACCAACCT | | TTTTATGC | | | -5255799 |
| Region 1 | | | | | | |
| Non- | AGCCTTGTCCTCC | 80 | AAACGGTCCCT | 90 | 248 | chr11:5271005 |
| Target | TCTGTGA | | GGCTAAACT | | | -5271252 |
| Region 2 | | | | | | |

TABLE 11
Haplotype PCR primers used in this study

| | | SEQ | | SEQ | Product | Genomic |
|---|---|---|---|---|---|---|
| Target | For- | ID | Re- | ID | size | coor- |
| region | ward | NO: | verse | NO: | (bp) | dinates |
| APOC3 | GCCT | 91 | ATCCTT | 92 | 4922 | chr11: |
| Enhancer- | CCTT | | GGCGGT | | | 116696647- |
| APOC3 | TTTG | | CTTGGT | | | 116701568 |
| Exon3 | ATGC | | G | | | |
| | AGCC | | | | | |

Tables 12-14: Amplicon Sequencing Primers Used in this Study

TABLE 12
1st PCR: Amplify region and add Overhangs

| | | | SEQ | | SEQ |
|---|---|---|---|---|---|
| | | | ID | | ID |
| | Target region | Forward | NO: | Reverse | NO: |
| genomic DNA | E1 (APOC3 locus) | ACACTCTTTCCC TACACGACGCTC TTCCGATCTGCC TCCTTTTTGATGCA GCC | 93 | 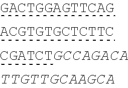 | 111 |
| | E2 (APOC3 locus) | ACACTCTTTCCC TACACGACGCTC TTCCGATCTGCT TGCAACAATGTCT GGCA | 94 | 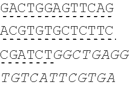 | 112 |
| | E3 (APOC3 locus) | ACACTCTTTCCC TACACGACGCTC TTCCGATCTGCT TGCAACAATGTCT GGCA | 95 | 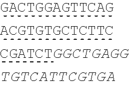 | 113 |
| | E4 (APOC3 locus) | ACACTCTTTCCC TACACGACGCTC TTCCGATCTGAA TCAGGCACAGTCC AGCT | 96 | 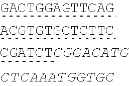 | 114 |
| | E5 (APOC3 locus) | ACACTCTTTCCC TACACGACGCTC TTCCGATCTTGA GCATGTCCGAGAG CATC | 97 | 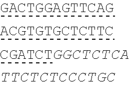 | 115 |
| | E6 (APOC3 locus) | ACACTCTTTCCC TACACGACGCTC TTCCGATCTGCA GGGAGAGAATGAG AGCC | 98 | 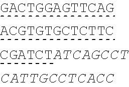 | 116 |
| | E0 (APOC3 locus) | ACACTCTTTCCC TACACGACGCTC TTCCGATCTATCT CAGCCCCGAGAAG G | 99 | 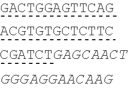 | 117 |
| | P (APOC3 promoter) | ACACTCTTTCCC TACACGACGCTC TTCCGATCTGTT CCTGAGCTCATCT GGGC | 100 | 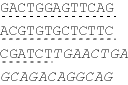 | 118 |
| | APOC3 exon 3 region | ACACTCTTTCCC TACACGACGCTC TTCCGATCTACT CCTTGTTGTTGCC CTCC | 101 | 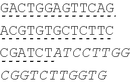 | 119 |
| cDNA | APOC3 exon 3 region | ACACTCTTTCCC TACACGACGCTC TTCCGATCTCTCT GCCCGAGCTTCAG AG | 102 | 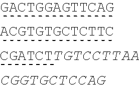 | 120 |
| ChIP/ Input DNA | E1 (APOC3 locus) | ACACTCTTTCCC TACACGACGCTC TTCCGATCTATG CTGCCTCCTTTTTG ATG | 103 | 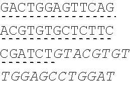 | 121 |
| | E2 (APOC3 locus) | ACACTCTTTCCC TACACGACGCTC TTCCGATCTCGT ACTGCCTGTGTGT CCTT | 104 | 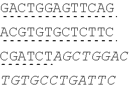 | 122 |
| | E3 (APOC3 locus) | ACACTCTTTCCC TACACGACGCTC TTCCGATCTGTG CTTGCAACAATGTC TGG | 105 | 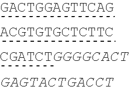 | 123 |
| | E4 (APOC3 locus) | ACACTCTTTCCC TACACGACGCTC TTCCGATCTAGA GGGGAGGAGGAG ACTGA | 106 | 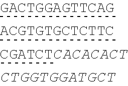 | 124 |
| | E5 (APOC3 locus) | ACACTCTTTCCC TACACGACGCTC TTCCGATCTATTT GAGCATGTCCGAG AGC | 107 | 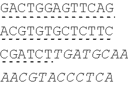 | 125 |
| | E6 (APOC3 locus) | ACACTCTTTCCC TACACGACGCTC TTCCGATCTAGG GTGCAAGTAGCTG ATGG | 108 | 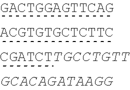 | 126 |
| | E0 (APOC3 locus) | ACACTCTTTCCC TACACGACGCTC TTCCGATCTATCT CAGCCCCGAGAAG G | 109 | 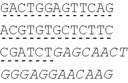 | 127 |
| | P (APOC3 promoter) | ACACTCTTTCCC TACACGACGCTC TTCCGATCTCTC ATCTCCACTGGTC AGCA | 110 | 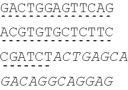 | 128 |

BOLD: next generation sequencing forward adapter sequences 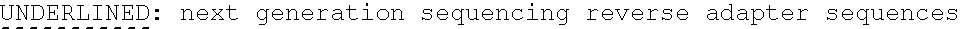
ITALICS: Annealing portion

TABLE 13
2nd PCR: Add indices and p5/p7

| | | SEQ | | SEQ |
|---|---|---|---|---|
| | Index | ID | | ID |
| Index | Sequence | NO: | Forward | NO: |
| i501 | TATAGCCT | 129 | AATGATACGGCGACCACCGAGATCTACA | 145 |
| | | | CTATAGCCTACACTCTTTCCCTACACGA | |
| | | | CGCTCTTCCGATCT | |
| i502 | ATAGAGGC | 130 | AATGATACGGCGACCACCGAGATCTACA | 146 |
| | | | CATAGAGGCACACTCTTTCCCTACACGA | |
| | | | CGCTCTTCCGATCT | |
| i503 | CCTATCCT | 131 | AATGATACGGCGACCACCGAGATCTACA | 147 |
| | | | CCCTATCCTACACTCTTTCCCTACACGA | |
| | | | CGCTCTTCCGATCT | |
| i504 | GGCTCTGA | 132 | AATGATACGGCGACCACCGAGATCTACA | 148 |
| | | | CGGCTCTGAACACTCTTTCCCTACACGA | |
| | | | CGCTCTTCCGATCT | |
| i505 | AGGCGAAG | 133 | AATGATACGGCGACCACCGAGATCTACA | 149 |
| | | | CAGGCGAAGACACTCTTTCCCTACACG | |
| | | | ACGCTCTTCCGATCT | |
| i506 | TAATCTTA | 134 | AATGATACGGCGACCACCGAGATCTACA | 150 |
| | | | CTAATCTTAACACTCTTTCCCTACACGA | |
| | | | CGCTCTTCCGATCT | |
| i507 | CAGGACGT | 135 | AATGATACGGCGACCACCGAGATCTACA | 151 |
| | | | CCAGGACGTACACTCTTTCCCTACACG | |
| | | | ACGCTCTTCCGATCT | |
| i508 | GTACTGAC | 136 | AATGATACGGCGACCACCGAGATCTACA | 152 |
| | | | CGTACTGACACACTCTTTCCCTACACGA | |
| | | | CGCTCTTCCGATCT | |
| J5001 | AACGGTTG | 137 | AATGATACGGCGACCACCGAGATCTACA | 153 |
| | | | CAACGGTTGACACTCTTTCCCTACACGA | |
| | | | CGCTCTTCCGATCT | |
| J5002 | CTGTTCTA | 138 | AATGATACGGCGACCACCGAGATCTACA | 154 |
| | | | CCTGTTCTAACACTCTTTCCCTACACGA | |
| | | | CGCTCTTCCGATCT | |
| J5003 | CATTGTAA | 139 | AATGATACGGCGACCACCGAGATCTACA | 155 |
| | | | CCATTGTAAACACTCTTTCCCTACACGA | |
| | | | CGCTCTTCCGATCT | |
| J5004 | CTCCTCGA | 140 | AATGATACGGCGACCACCGAGATCTACA | 156 |
| | | | CCTCCTCGAACACTCTTTCCCTACACGA | |
| | | | CGCTCTTCCGATCT | |
| J5005 | ACTCGCCT | 141 | AATGATACGGCGACCACCGAGATCTACA | 157 |
| | | | CACTCGCCTACACTCTTTCCCTACACGA | |
| | | | CGCTCTTCCGATCT | |
| J5006 | CAGTTGTT | 142 | AATGATACGGCGACCACCGAGATCTACA | 158 |
| | | | CCAGTTGTTACACTCTTTCCCTACACGA | |
| | | | CGCTCTTCCGATCT | |
| J5007 | AGAGATCC | 143 | AATGATACGGCGACCACCGAGATCTACA | 159 |
| | | | CAGAGATCCACACTCTTTCCCTACACGA | |
| | | | CGCTCTTCCGATCT | |
| J5008 | TGGCACTT | 144 | AATGATACGGCGACCACCGAGATCTACA | 160 |
| | | | CTGGCACTTACACTCTTTCCCTACACGA | |
| | | | CGCTCTTCCGATCT | |

TABLE 14
2nd PCR: Add indices and p5/p7

| | | SEQ | | SEQ |
|---|---|---|---|---|
| | Index | ID | | ID |
| Index | Sequence | NO: | Reverse | NO: |
| i701 | ATTACTCG | 161 | 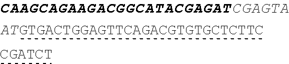 | 185 |
| i702 | TCCGGAGA | 162 | 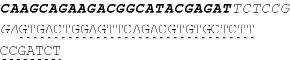 | 186 |
| i703 | CGCTCATT | 163 | 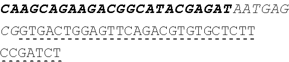 | 187 |
| i704 | GAGATTCC | 164 | 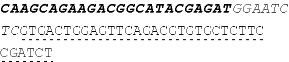 | 188 |
| i705 | ATTCAGAA | 165 | 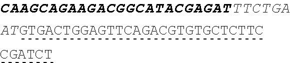 | 189 |
| i706 | GAATTCGT | 166 | 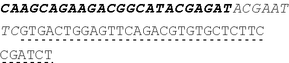 | 190 |
| i707 | CTGAAGCT | 167 | 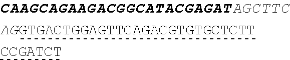 | 191 |
| i708 | TAATGCGC | 168 | 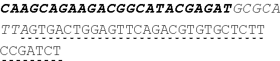 | 192 |
| i709 | CGGCTATG | 169 | 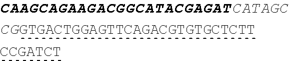 | 193 |
| i710 | TCCGCGAA | 170 | 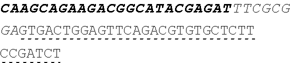 | 194 |
| i711 | TCTCGCGC | 171 | 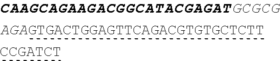 | 195 |
| i712 | AGCGATAG | 172 | 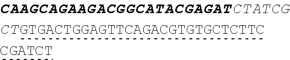 | 196 |
| J7001 | ATCCTATC | 173 | 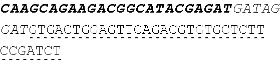 | 197 |
| J7002 | TCGTGTCA | 174 | 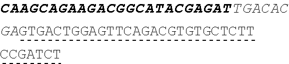 | 198 |
| J7003 | CTGTACTA | 175 | 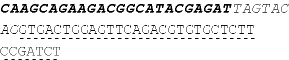 | 199 |
| J7004 | GTCGTCTA | 176 | 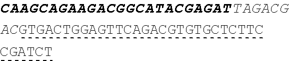 | 200 |
| J7005 | GTATTCCA | 177 | 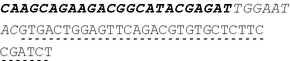 | 201 |
| J7006 | GGCAGACC | 178 | 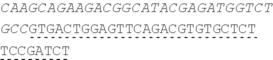 | 202 |
| J7007 | AGTATAAT | 179 | 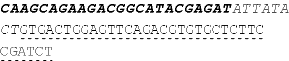 | 203 |
| J7008 | GAACGTGC | 180 | 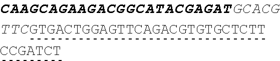 | 204 |
| J7009 | GTGGAGAT | 181 | 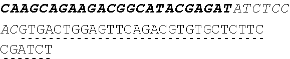 | 205 |
| J7010 | TGTATCTT | 182 | 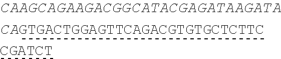 | 206 |
| J7011 | ATCTGGAA | 183 | 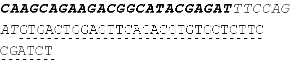 | 207 |
| J7012 | TAAGGAGG | 184 | 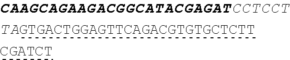 | 208 |

Note:
BOLD = D500B; 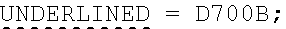
ITALICS = Index;
PLAIN = p5;

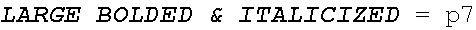

### REFERENCES

1 Pickar-Oliver, A. & Gersbach, C. A. The next generation of CRISPR-Cas technologies and applications. Nat Rev Mol Cell Biol 20, 490-507, doi:10.1038/s41580-019-0131-5 (2019).
2 Thakore, P. I., Black, J. B., Hilton, I. B. & Gersbach, C. A. Editing the epigenome: technologies for programmable transcription and epigenetic modulation. Nat Methods 13, 127-137, doi:10.1038/nmeth.3733 (2016).
3 Wang, H., La Russa, M. & Qi, L. S. CRISPR/Cas9 in Genome Editing and Beyond. Annu Rev Biochem 85, 227-264, doi:10.1146/annurev-biochem-060815-014607 (2016).
4 Hilton, I. B. et al. Epigenome editing by a CRISPR-Cas9-based acetyltransferase activates genes from promoters and enhancers. Nat Biotechnol 33, 510-517, doi:10.1038/nbt.3199 (2015).
5 Benabdallah, N. S. et al. Decreased Enhancer-Promoter Proximity Accompanying Enhancer Activation. Mol Cell, doi:10.1016/j.molcel.2019.07.038 (2019).
6 Tak, Y. E. et al. Inducible and multiplex gene regulation using CRISPR-Cpf1-based transcription factors. Nat Methods 14, 1163-1166, doi:10.1038/nmeth.4483 (2017).
7 Simeonov, D. R. et al. Discovery of stimulation-responsive immune enhancers with CRISPR activation. Nature 549, 111-115, doi:10.1038/nature23875 (2017).
8 Laguna, T. et al. New insights on the transcriptional regulation of CD69 gene through a potent enhancer located in the conserved non-coding sequence 2. Mol Immunol 66, 171-179, doi:10.1016/j.molimm.2015.02.031 (2015).
9 Chen, J. C. & Goldhamer, D. J. The core enhancer is essential for proper timing of MyoD activation in limb buds and branchial arches. Dev Biol 265, 502-512, doi:10.1016/j.ydbio.2003.09.018 (2004).
10 Ktistaki, E., Lacorte, J. M., Katrakili, N., Zannis, V. I. & Talianidis, I. Transcriptional regulation of the apolipoprotein A-IV gene involves synergism between a proximal orphan receptor response element and a distant enhancer located in the upstream promoter region of the apolipoprotein C-III gene. Nucleic Acids Res 22, 4689-4696, doi:10.1093/nar/22.22.4689 (1994).
11 Zannis, V. I., Kan, H. Y., Kritis, A., Zanni, E. E. & Kardassis, D. Transcriptional regulatory mechanisms of the human apolipoprotein genes in vitro and in vivo. Curr Opin Lipidol 12, 181-207, doi:10.1097/00041433-200104000-00012 (2001).
12 Liu, P. Q. et al. Regulation of an endogenous locus using a panel of designed zinc finger proteins targeted to accessible chromatin regions. Activation of vascular endothelial growth factor A. J Biol Chem 276, 11323-11334, doi:10.1074/jbc.M011172200M011172200 [pii] (2001).
13 Maeder, M. L. et al. Robust, synergistic regulation of human gene expression using TALE activators. Nat Methods 10, 243-245, doi:10.1038/nmeth.2366nmeth.2366 [pii] (2013).
14 Perez-Pinera, P. et al. RNA-guided gene activation by CRISPR-Cas9-based transcription factors. Nat Methods 10, 973-976, doi:10.1038/nmeth.2600nmeth.2600 [pii] (2013).
15 Perez-Pinera, P. et al. Synergistic and tunable human gene activation by combinations of synthetic transcription factors. Nat Methods 10, 239-242, doi:10.1038/nmeth.2361nmeth.2361 [pii] (2013).
16 Maeder, M. L. et al. CRISPR RNA-guided activation of endogenous human genes. Nat Methods 10, 977-979, doi:10.1038/nmeth.2598nmeth.2598 [pii] (2013).
17 Chavez, A. et al. Comparison of Cas9 activators in multiple species. Nat Methods 13, 563-567, doi:10.1038/nmeth.3871 (2016).
18 Wienert, B., Martyn, G. E., Funnell, A. P. W., Quinlan, K. G. R. & Crossley, M. Wake-up Sleepy Gene: Reactivating Fetal Globin for beta-Hemoglobinopathies. Trends Genet 34, 927-940, doi:10.1016/j.tig.2018.09.004 (2018).
19 Diepstraten, S. T. & Hart, A. H. Modelling human haemoglobin switching. Blood Rev 33, 11-23, doi:10.1016/j.blre.2018.06.001 (2019).
20 Sankaran, V. G. & Orkin, S. H. The switch from fetal to adult hemoglobin. Cold Spring Harb Perspect Med 3, a011643, doi:10.1101/cshperspect.a011643 (2013).
21 Sankaran, V. G. et al. Human fetal hemoglobin expression is regulated by the developmental stage-specific repressor BCL11A. Science 322, 1839-1842, doi:10.1126/science.1165409 (2008).
22 Li, Q., Harju, S. & Peterson, K. R. Locus control regions: coming of age at a decade plus. Trends Genet 15, 403-408, doi:10.1016/s0168-9525(99)01780-1 (1999).
23 Zhou, D., Pawlik, K. M., Ren, J., Sun, C. W. & Townes, T. M. Differential binding of erythroid Krupple-like factor to embryonic/fetal globin gene promoters during development. J Biol Chem 281, 16052-16057, doi:10.1074/jbc.M601182200 (2006).
24 Liu, N. et al. Direct Promoter Repression by BCL11A Controls the Fetal to Adult Hemoglobin Switch. Cell 173, 430-442 e417, doi:10.1016/j.cell.2018.03.016 (2018).
25 Kleinstiver, B. P. et al. Engineered CRISPR-Cas12a variants with increased activities and improved targeting ranges for gene, epigenetic and base editing. Nat Biotechnol 37, 276-282, doi:10.1038/s41587-018-0011-0 (2019).
26 Cavalli, M. et al. Allele-specific transcription factor binding to common and rare variants associated with disease and gene expression. Hum Genet 135, 485-497, doi:10.1007/s00439-016-1654-x (2016).
27 Spisak, S. et al. CAUSEL: an epigenome- and genome-editing pipeline for establishing function of noncoding GWAS variants. Nat Med, doi:10.1038/nm.3975nm.3975 [pii] (2015).
28 Bailey, S. D. et al. ZNF143 provides sequence specificity to secure chromatin interactions at gene promoters. Nat Commun 2, 6186, doi:10.1038/ncomms7186 (2015).
29 Lek, M. et al. Analysis of protein-coding genetic variation in 60,706 humans. Nature 536, 285-291, doi:10.1038/nature19057 (2016).
30 Cooper, D. N., Krawczak, M., Polychronakos, C., Tyler-Smith, C. & Kehrer-Sawatzki, H. Where genotype is not predictive of phenotype: towards an understanding of the molecular basis of reduced penetrance in human inherited disease. Hum Genet 132, 1077-1130, doi:10.1007/s00439-013-1331-2 (2013).
31 Veitia, R. A., Caburet, S. & Birchler, J. A. Mechanisms of Mendelian dominance. Clin Genet 93, 419-428, doi:10.1111/cge.13107 (2018).
32 Matharu, N. et al. CRISPR-mediated activation of a promoter or enhancer rescues obesity caused by haploinsufficiency. Science 363, doi:10.1126/science.aau0629 (2019).
33 Dang, V. T., Kassahn, K. S., Marcos, A. E. & Ragan, M. A. Identification of human haploinsufficient genes and their genomic proximity to segmental duplications. Eur J Hum Genet 16, 1350-1357, doi:10.1038/ejhg.2008.111 (2008).
34 Liang, J. R., Lingeman, E., Ahmed, S. & Corn, J. E. Atlastins remodel the endoplasmic reticulum for selective autophagy. J Cell Biol 217, 3354-3367, doi:10.1083/jcb.201804185 (2018).
35 Wang, Y. et al. The 3D Genome Browser: a web-based browser for visualizing 3D genome organization and long-range chromatin interactions. Genome Biol 19, 151, doi:10.1186/s13059-018-1519-9 (2018).
36 Rao, S. S. et al. A 3D map of the human genome at kilobase resolution reveals principles of chromatin looping. Cell 159, 1665-1680, doi:10.1016/j.cell.2014.11.021 (2014).
37 Mikkelsen, T. S. et al. Genome-wide maps of chromatin state in pluripotent and lineage-committed cells. Nature 448, 553-560, doi:10.1038/nature06008 (2007).
38 Rohland, N. & Reich, D. Cost-effective, high-throughput DNA sequencing libraries for multiplexed target capture. Genome Res 22, 939-946, doi:10.1101/gr.128124.111 (2012).
40 Li, H. & Durbin, R. Fast and accurate short read alignment with Burrows-Wheeler transform. Bioinformatics 25, 1754-1760, doi:10.1093/bioinformatics/btp324btp324 [pii] (2009).
41 Corces, M. R. et al. An improved ATAC-seq protocol reduces background and enables interrogation of frozen tissues. Nat Methods 14, 959-962, doi:10.1038/nmeth.4396 (2017).
42 Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat Methods 10, 1213-1218, doi:10.1038/nmeth.2688 (2013).
43 Quinlan, A. R. & Hall, I. M. BEDTools: a flexible suite of utilities for comparing genomic features. Bioinformatics 26, 841-842, doi:10.1093/bioinformatics/btq033btq033 [pii] (2010).
44 Clement, K. et al. CRISPResso2 provides accurate and rapid genome editing sequence analysis. Nat Biotechnol 37, 224-226, doi:10.1038/s41587-019-0032-3 (2019).
45 Li, B., Kadura, I., Fu, D. J. & Watson, D. E. Genotyping with TaqMAMA. Genomics 83, 311-320, doi:10.1016/j.ygeno.2003.08.005 (2004).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An artificial transcription factor (aTF) system comprising:
(a) one or more enhancer-targeting aTF(s); and
(b) one or more promoter-targeting aTF(s).

2. The aTF system of claim 1, wherein the enhancer-targeting aTF(s) comprise:
(a) a fusion protein comprising a catalytically inactive Cas9 or catalytically inactive Cpf1 and a gene expression modulating domain; and
(b) a gRNA comprising a sequence complementary to a target gene enhancer sequence.

3. The aTF system of claim 1, wherein the enhancer-targeting aTF(s) comprises:
(a) a first fusion protein comprising a catalytically inactive Cas9 or catalytically inactive Cpf1 and a first dimerization domain;
(b) a second fusion protein comprising a gene expression modulating domain and a second dimerization domain; and
(c) a gRNA comprising a sequence complementary to a target gene enhancer sequence.

4. The aTF system of claim 1, wherein the promoter-targeting aTF(s) comprises:
(a) a fusion protein comprising a catalytically inactive Cas9 or catalytically inactive Cpf1 and a gene expression modulating domain; and
(b) a gRNA comprising a sequence complementary to a target gene promoter sequence.

5. The aTF system of claim 1, wherein the promoter-targeting aTF(s) comprises:
(a) a first fusion protein comprising a catalytically inactive Cas9 or catalytically inactive Cpf1 and a first dimerization domain;
(b) a second fusion protein comprising a gene expression modulating domain and a second dimerization domain; and
(c) a gRNA comprising a sequence complementary to a target gene promoter sequence.

6. An artificial transcription factor (aTF) system comprising:
(a) a fusion protein comprising a catalytically inactive Cas9 or catalytically inactive Cpf1 and a gene expression modulating domain;
(b) a first gRNA comprising a sequence complementary to a target gene enhancer sequence; and
(c) a second gRNA comprising a sequence complementary to a target gene promoter sequence.

7. An artificial transcription factor (aTF) system comprising:
(a) a first fusion protein comprising a catalytically inactive Cas9 or catalytically inactive Cpf1 and a first dimerization domain;
(b) a second fusion protein comprising a gene expression modulating domain and a second dimerization domain;
(c) a first gRNA comprising a sequence complementary to a target gene enhancer sequence; and
(d) a second gRNA comprising a sequence complementary to a target gene promoter sequence.

8. An artificial transcription factor (aTF) system comprising:
(a) a fusion protein comprising a catalytically inactive Cas9 or catalytically inactive Cpf1 and a gene expression modulating domain;
(b) a first gRNA comprising a sequence complementary to a target gene enhancer sequence; and
(c) a plurality of gRNAs each comprising a sequence complementary to a different target gene promoter sequence.

9. An artificial transcription factor (aTF) system comprising:
(a) a first fusion protein comprising a catalytically inactive Cas9 or catalytically inactive Cpf1 and a first dimerization domain;
(b) a second fusion protein comprising a gene expression modulating domain and a second dimerization domain;
(c) a first gRNA comprising a sequence complementary to a target gene enhancer sequence; and
(d) a plurality of gRNAs each comprising a sequence complementary to a different target gene promoter sequence.

10. The aTF system of claim 3 wherein the first dimerization domain comprises DmrA and the second dimerization domain comprises DmrC.

11. The aTF system of claim 3, further comprising a dimerization agent.

12. The aTF system of claim 2, wherein the gene expression modulating domain is an activation domain selected from the group consisting of p65, VPR, VPR64, p300, and combinations thereof.

13. The aTF system of claim 2, wherein the gene expression modulating domain comprises: (1) a protein that can introduce or remove covalent modifications to histones or DNA, optionally LSD1 or TET1; or (2) a protein that directly or indirectly recruits other proteins in the cell that in turn can modulate gene expression.

14. The aTF system of claim 2, wherein the enhancer-targeting aTF, the promoter-targeting aTF, or both each comprises two or more gene expression modulating domains.

15. The aTF system of claim 1 further comprising a drug that induces the activity of the enhancer-targeting aTF(s) and/or the promoter-targeting aTF(s).

16. The aTF system of claim 1, wherein
the target gene enhancer sequence comprises two or more alleles and the enhancer-targeting aTF comprises a programmable DNA binding domain specific for a subset of the alleles; and/or
the target gene promoter sequence comprises two or more alleles and the promoter-targeting aTF comprises a programmable DNA binding domain specific for a subset of the alleles.

17. The aTF system of claim 2, wherein
the target gene enhancer sequence comprises two or more alleles and the gRNA is specific for a subset of the alleles; and/or
the promoter gene enhancer sequence comprises two or more alleles and the gRNA is specific for a subset of the alleles.

18. The aTF system of claim 2, wherein the target gene is selected from the group consisting of IL2RA, MYOD1, CD69, HBB, HBE, HBG1/2, APOC3, APOA4 and combinations thereof.

19. A vector comprising nucleic acid sequences encoding one or more of the components of the aTF system of claim 1.

20. A cell comprising the vector of claim 19.

21. A pharmaceutical composition comprising the aTF system of claim 1 and a pharmaceutically acceptable carrier.

22. A method for modulating target gene expression in a cell, the method comprising contacting the cell with the aTF system of claim 1.

23. A method for allele-specific modulation of a target gene expression in a cell, the method comprising contacting the cell with the aTF system of claim 16.

24. A method for treating or preventing a condition or disease in a subject, the method comprising contacting the cell with the aTF system of claim 1.

25. The method of claim 25, wherein the condition or disease is caused, at least in part, by insufficient expression of the target gene or the adverse effect of a mutant allele.
